(19) United States
(12) Patent Application Publication
(10) Pub. No.: US 2021/0275094 A1
(43) Pub. Date: Sep. 9, 2021

(54) SYSTEM, APPARATUS, AND METHOD OF ACCELERATED TRAINING FOR PERFORMANCE UNDER STRESS

(71) Applicant: ACCENTUS-LUDUS, LLC, Minneapolis, MN (US)

(72) Inventor: Marcus Wynne, Box Elder, SD (US)

(21) Appl. No.: 17/320,478

(22) Filed: May 14, 2021

Related U.S. Application Data
(60) Provisional application No. 63/138,155, filed on Jan. 15, 2021.

(51) Int. Cl. A61B 5/00; G09B 9/00

(52) U.S. Cl. CPC A61B 5/4884; G09B 9/00

(57) ABSTRACT

A method, apparatus, and system for improving performance in stressful environments include presenting a series of instructions and a series of simulated environments within a brief period of time to a trainee. In each simulated environment, the trainee is tasked with achieving a desired outcome requiring the use of a new skill under stress. Stress is increased throughout the training protocol. Each skill may be a critical skill along a critical skill path. A simulated environment may be designed to favor a successful outcome. A trainee may be monitored for markers that represent manifestations of psychophysiological states as the trainee learns or uses a new skill under stress. The markers may be stored and used to verify appropriate conditions for subconscious installation of critical skills in the same simulation, in subsequent simulations, in subsequent training protocols, for subsequent trainees, or on-demand in real-time as needed.

### RELATED APPLICATION

This application claims the benefit of U.S. Provisional Application No. 63/138,155 filed on Jan. 15, 2021, which is hereby incorporated herein by reference in its entirety.

### TECHNICAL FIELD

This disclosure relates to improving performance in stressful environments. In particular, this disclosure relates to systems and methods for monitoring for and using markers of learning and performance while installing skills under stress to determine critical markers for enhancing training and performance.

### BACKGROUND

Many organizations have made efforts to train individuals to recall and perform skills on the job. In particular, some organizations have made substantial efforts to improve the retention and performance of skills learned in training when faced with complex, high-stress environments, which may include immediate, threat-to-life situations. A goal of such efforts may be, for example, protecting and saving lives. Successful performance of skills in, for example, immediate threat-to-life situations often requires effective decision-making. However, high-stress presented in such situations may trigger a strong emotional response in many individuals, which may interfere with effective decision-making and perception and, thus, may compromise successful performance of skills learned in training.

Situational awareness has been identified as a key characteristic to successful performance in complex, stressful environments. Situational awareness may be described as the perception of elements in an environment, the comprehension of their meaning, and the ability to make decisions about like future events in the environment. In other words, situational awareness requires accurate processing of perception, comprehension, and projection for appropriate actions to take place leading to successful performance. Through research, concepts such as psychophysiological state management, stress inoculation, and cognitive strategy have been recognized for improving situational awareness.

Some stress inoculation training protocols have been developed to aid trainees in coping with stress during and after exposure to a stressful environment. Examples of stress inoculation training protocols include teaching about the nature of stress, training to recognize maladaptive cognition, and using recognition to cue adaptive coping techniques. In many settings, stress inoculation training has been shown to improve the performance of tasks in stressful environments.

Some psychophysiological state (PPS) management protocols have been developed to help the awareness and management of psychophysiological responses and the recovery from such high-stress exposure. An example of a PPS management protocol includes mindfulness-based mind fitness training (MMFT). In many settings, PPS management training has been shown to improve attention capabilities and stress recovery.

Cognitive training protocols have been developed to better install cognitive and decision-making abilities in trainees, for example, to reduce the frequency and range of perceptual errors and to improve memory. Examples of cognitive training include basic response inhibition techniques (stopping a prepotent motor response) and field of view attention training. In many settings, cognitive training has been shown to improve accuracy in targeting to reduce collateral mistakes, to improve perceptual recognition of hazards, and to reduce errors in driving.

In the arena of training, multiple skills must be learned and deployed to successfully perform in a high-stress environment. Limited time and resources often dictate that an entire training protocol be confined to a few days, or even a few hours. Existing accelerated learning techniques have been developed to address the short time available to train individuals. For example, an accelerated learning protocol may be developed to train motor skills having both basic and advanced components. However, existing accelerated learning techniques do not address compromised performance due to exposure to complex, high-stress environments.

Generally, stress has been recognized as having several negative effects on the cognitive processing of an event, including receiving sensory information about an individual's environment and related decision-making. Stress may cause attention to narrow and contribute to a lack of concentration. Furthermore, perception of events may become distorted, resulting in misinterpretation and even hallucination in some severe cases. These effects on attention and perception may affect the information provided to memory and thinking and decision-making processes. Further, stress may hinder the memory recall process by repressing or distorting information, which may affect thinking and decision-making processes, which may use real-time sensory information and recalled information from memory. Further still, thinking and decision-making processes themselves may become susceptible to wishful thinking, delusions, statistical errors, and cognitive dissonance. At bottom, stress may contribute to decisions that are less effective and different than decisions that would be made without stress and may be considered inappropriate or lacking competence. High stress, such as immediate threat-to-life stress in particular, may elevate these negative effects.

Each of the concepts of situational awareness—psychophysiological state management, stress inoculation, and cognitive ability—has been found to contribute to an individual's retention of skills, performance of skills, and stress coping in at least some stressful environments. However, there remains a need for improved training protocols that integrate accelerated learning and improved situational awareness techniques to address performance under stress, particularly immediate, threat-to-life stress.

Even when a training protocol is capable of accelerated and effective installation, the development and deployment of the training protocol is time intensive and labor intensive. A single trainer preferably develops every aspect of the training protocol, from interviewing to skill sequencing to stressor and simulation design, for example. For example, the trainee may have to understand the skills needed to complete the objective, to understand the trainee population, to test the training protocol on a sample set of trainees before deployment, and to appropriately revise the training protocol to improve results prior to deploying the training protocol to the trainee population. Some drawbacks of this method include the intensive time required to carry out these development tasks, as well as the development of the specialized skill set in the trainer through experience.

Moreover, once the training protocol has been developed, often by a single trainer, only one trainer may have the requisite knowledge to deploy the training protocol, or in other words, to carry out the installation protocol with trainees for installation. The single-trainer bottleneck may be a problem when desiring to deploy the training protocol to trainees in distributed geographic locations in a short time period. One solution is to train other facilitators or trainers in conducting the training protocol, for example, to reduce the deployment time once the trainers are sufficiently trained. However, installing the required skill set in the trainers often requires even more time than the training protocol itself, which adds a step to the overall process.

Training has been shown to be enhanced with monitoring and feedback equipment has been developed to enhance training. One example can be found in U.S. Pat. No. 9,173,582, entitled “Adaptive Performance Trainer.” With such equipment, expert's biomarkers are monitored and incorporated into equipment as targets. Then, as a trainee's biomarkers are monitored during training, feedback is given to the trainee to utilize skills to improve performance. One drawback of this process is the conscious feedback to activate a skill, which may be hindered by stress. Another drawback is that the described process does not provide insight into the learning process under stress, particularly subconscious installation.

### SUMMARY

The present disclosure relates to training protocols for improving performance in stressful environments.

Some particular aspects of the present disclosure relate to, among other things, a method of training to install a sequence of skills under simulated stress designed to improve performance of the skills in stressful environments. The training, in various aspects, integrates accelerated learning with psychophysiological state management, stress inoculation, and cognitive skills training. The method may be described as a training protocol, which includes several simulated environments to be introduced to a trainee in quick succession designed to subconsciously install a set of critical skills in an accelerated manner that will be retained for at least partially subconscious recall and use in sequence along a critical skill path.

In various illustrative embodiments, a method for installing a critical skill path in a trainee is disclosed herein. The critical skill path may comprise one or more critical skills for successfully achieving an outcome in a stressful environment, which may likely elicit one or more stress response states in the trainee. The method comprises presenting a first instruction to the trainee, prompting the trainee to perform a first task using an essential critical skill of the critical skill path. The method further comprises introducing the trainee into a first simulated environment, within a first brief period of time after presenting the first instruction, exposing the trainee to a first stressor and requiring the trainee to use the essential critical skill to successfully perform the first task. The first stressor may be designed to elicit a first stress response state in the trainee. The first simulated environment may be designed to favor successful performance of the first task by the trainee in the first stress response state. Yet further, the method comprises presenting a second instruction to the trainee, within a second brief period of time after completing the first task, prompting the trainee to perform a second task using the essential critical skill and at least another critical skill of the critical skill path. Still further, the method comprises introducing the trainee into a second simulated environment, within a third brief period of time after presenting the second instruction, exposing the trainee to a second stressor and requiring the trainee to use the essential critical skill and the at least another critical skill to successfully perform the second task. The second stressor may be designed to elicit a second stress response state in the trainee more stressful than the first stress response state. The second simulated environment may be designed to favor successful performance of the second task by the trainee in the second stress response state. In addition, the method comprises continuing to present one or more additional instructions to the trainee, within a brief period of time after completing an immediately preceding task, each additional instruction prompting the trainee to perform a task and to use a critical skill set including the critical skills used in the immediately preceding task and at least one additional critical skill, until all critical skills in the critical skill path have been used in performing the immediately preceding task. Furthermore, the method also comprises continuing to introduce the trainee into one or more additional simulated environments within a brief period of time after presenting each additional instruction until all critical skill in the critical skill path have been used in performing the immediately preceding task, each additional simulated environment exposing the trainee to a stressor designed to elicit a stress response state in the trainee more stressful than the immediately preceding stress response state and requiring the trainee to use the corresponding critical skill set to successfully perform the task as instructed. Each additional simulated environment may be designed to favor successful performance of the task, as instructed, by the trainee in the stress response state.

An illustrative method facilitates rapid installation the critical skill path in the trainee under simulated stress for effective performance in immediate threat-to-life situations, for example, by enhancing situational awareness capabilities in the trainee. Situational awareness may be enhanced by installation into and recall from of required skills the subconscious of the trainee. The trainee may also dedicate less time to training to achieve the similar or better performance in complex, high-stress environments than other non-integrated training methods. The illustrative training method may be useful a broad range of situations, which may not be life threatening. A trainee may gain enhanced situational awareness, and thus performance, in environments including some stress or some complexity via a method of this disclosure.

Some particular aspects of the present disclosure relate to, among other things, methods, apparatuses, and systems for enhancing accelerated training to subconsciously install a skill to improve performance of the skill in a stressful environment. The accelerated training, in various aspects, integrates accelerated learning with psychophysiological state management, stress inoculation, and cognitive skills training for delivery by an autonomous, artificial tutor. A simulated environmental factor may be presented to a trainee to facilitate the subconscious installation of a skill in an accelerated manner that will be available for use or retained for later use. The accelerated training is enhanced, in various aspects, by utilizing various markers of learning, which may be internal biomarkers or external cues. Markers may be measured during a training protocol of a trainee. A critical marker may be derived representing a cognitive or neurological change associated with learning a skill. A sequence of markers for learning may be associated with a critical sequence of skills for successful performance. The critical markers may be used to make adjustments to the same training protocol in which markers are measured or even may be used to provide insight into a generalized learning process based on one or more trainees. In one example, determining the sequence of markers may facilitate the development and deployment of an adaptable or automated training protocol. In another example, the sequence of markers may facilitate the development of a “learning under stress” model or algorithm that can be generally applied to enhancing training development and deployment, as well as to developing future aspects of man-machine interfacing, enhanced cognition, and artificial intelligence.

In various illustrative embodiments, a method for developing a learning under stress model comprises introducing a trainee into a simulated environment exposing the trainee to a stressor and requiring the trainee to use a skill to successfully perform a task, wherein the stressor is designed to elicit a stress response state in the trainee, and wherein the simulated environment is designed to favor successful performance of the task by the trainee in the stress response state; monitoring the trainee for a marker related to the simulated environment; and determining a critical marker for the learning under stress model in response to the marker monitoring step.

In some illustrative embodiments, a method of using a learning under stress model comprises introducing a trainee into a simulated environment exposing the trainee to an environmental factor designed to facilitate the presence of a critical marker, the environ mental factor including a stressor and requiring the trainee to use a skill to successfully perform a task, wherein the stressor is designed to elicit a stress response state in the trainee, and wherein the simulated environment is designed to favor successful performance of the task by the trainee in the stress response state; monitoring the trainee for a critical marker defined in response to monitoring a previous trainee; determining whether the critical marker is present or absent in response to the monitoring step; and determining a subsequent environmental factor in response to the step of determining the absence of the critical marker, the subsequent environmental factor being different than the environmental factor and designed to further facilitate the presence of the critical marker.

In further illustrative embodiments, an apparatus for developing a learning under stress model for installing a critical skill with a critical skill presentation comprises a data storage configured to store one or more parameters of the learning under stress model associated with the critical skill including at least simulated environmental factor and a critical marker target; a sensor configured to provide a marker signal representing a specific measured marker of a trainee after being introduced into an environment exposing the trainee to a simulated environmental factor designed to facilitate subconscious installation of the critical skill in the trainee for achieving a desired outcome under stress; and a data processor. The data processor is configured to: determine whether the marker signal is associated with achievement of the desired outcome; determine whether the specific measured marker is a critical marker for the learning under stress model in response to the marker signal being associated with achievement of the desired outcome; and determine the critical marker target for storage in the data storage in response to determining that the measured marker is a critical marker, the critical marker target representing the a critical marker value associated with achievement of the desired outcome.

In additional illustrative embodiments, an apparatus for installing a critical skill with a critical skill presentation according to a learning under stress model comprises a data storage configured to store one or more parameters of the learning under stress model associated with the critical skill including at least a simulated environmental factor, a critical marker target for a critical marker, and a desired outcome marker representing achievement of a desired outcome in a specific environment; an installation controller configured to expose the trainee to the simulated environmental factor of the specific environment designed to facilitate subconscious installation of the critical skill in the trainee for achieving the desired outcome under stress; a sensor configured to provide a critical marker signal representing a measured value of a critical marker after introducing the critical skill presentation to the trainee; and a data processor. The data processor is configured to determine whether the critical marker is present or absent in response to the critical marker signal and the critical marker target in the data storage; and determine a subsequent simulated environmental factor for exposure to the trainee in response to the critical marker being absent, the subsequent simulated environmental factor being different than the simulated environmental factor in at least one of type, level, and number. The installation controller is further configured to expose the trainee to the subsequent simulated environmental factor in response to the data processor determining the subsequent simulated environmental factor.

In still further illustrative embodiments, an apparatus for using a learning under stress model to adaptively install a critical skill of an individual exposed to a stressful environment having one or more environmental factors comprises a data storage configured to store one or more parameters of the learning under stress model associated with the critical skill including at least: an environmental marker corresponding to a specific environmental factor, a critical marker target corresponding to a specific critical marker, a simulated environmental factor designed to facilitate subconscious installation of the critical skill in the individual for achieving a desired outcome under stress, and a critical skill presentation designed to introduce the critical skill to the individual for subconscious installation; an installation controller configured to expose the individual to the simulated environmental factor to facilitate installation of the critical skill in the individual and to present the critical skill presentation to the individual in the stressful environment; a marker sensor configured to provide a critical marker signal representing a measurement of the specific critical marker of the individual; an environmental sensor configured to provide an environmental signal representing a measurement of the specific environmental factor; and a data processor. The data processor is configured to determine whether to install the critical skill in response to the environmental signal and the environmental marker in the data storage; determine whether to present the critical skill presentation to the trainee in response to determining to install the critical skill; and determine whether to expose the trainee to the simulated environmental factor to facilitate subconscious installation of the critical skill in response to determining to install the critical skill and the critical marker signal. The installation controller is further configured to expose the trainee to the simulated environmental factor and to present the critical skill presentation in response to the respective data processor determinations.

Many illustrative embodiments facilitate rapid installation the critical skill path in the trainee under simulated stress for effective performance in immediate threat-to-life situations, for example, by enhancing situational awareness capabilities in the trainee. Situational awareness may be enhanced by installation into and recall from of required skills the subconscious of the trainee. The trainee may also dedicate less time to training to achieve the similar or better performance in complex, high-stress environments than other non-integrated training methods. The illustrative training method may be useful a broad range of situations, which may not be life threatening. A trainee may gain enhanced situational awareness, and thus performance, in environments including some stress or some complexity via a method of this disclosure. Furthermore, adaptable or automated training protocols may be developed and deployed in a relatively shorter amount of time than an accelerated training protocol by a single trainer, even on-demand, real-time training in a real-world environment. Moreover, insight provided into a “learning under stress” model or algorithm for the sequence of skills and the trainee, may be generalized to other skills and other trainees, for example, or even utilized to augment man-machine interfacing, enhanced cognition, and artificial intelligence.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The disclosure may be more completely understood in consideration of the following detailed description of various embodiments of the disclosure in connection with the accompanying drawings.

FIG. 1 is a schematic representation of an illustrative training method, according to various embodiments of the present disclosure.

FIG. 2 is a schematic representation of an illustrative series of experiences in the training method of FIG. 1.

FIG. 3 is a schematic representation of an illustrative critical skill path for installation by the training method of FIG. 1.

FIG. 4 is a schematic representation of an illustrative installation protocol of the training method of FIG. 1.

FIG. 5 is a schematic representation of an illustrative basic simulation phase of the installation protocol of FIG. 4.

FIG. 6 is a schematic representation of an illustrative core simulation phase of the installation protocol of FIG. 4.

FIG. 7 is a schematic representation of an illustrative stress coaching process of the installation protocol of FIG. 4.

FIG. 8 is a schematic representation of an illustrative critical decision process of the installation protocol of FIG. 4.

FIG. 9 is a schematic representation of an illustrative skill introduction process of the installation protocol of FIG. 4.

FIG. 10 is a schematic representation of an illustrative initial comprehensive simulation phase of the installation protocol of FIG. 4.

FIG. 11 is a schematic representation of an illustrative advanced comprehensive simulation phase of the installation protocol of FIG. 4.

FIG. 12 is a schematic representation of an illustrative escalating comprehensive simulation phase of the installation protocol of FIG. 4.

FIG. 13 is a schematic representation of an illustrative severe comprehensive simulation phase of the installation protocol of FIG. 4.

FIG. 14 is a schematic representation of an illustrative post-simulation presentation phase of the installation protocol of FIG. 4.

FIG. 15 is a schematic representation of at least one set of illustrative experiences according to the installation protocol of FIG. 4.

FIG. 16 is a schematic representation of an illustrative method for use with the installation protocol of FIG. 4 to ensure a threshold level of stress is experienced by the trainee.

FIG. 17 is a schematic representation of an illustrative marker monitoring apparatus for use with the training method of FIG. 1.

FIG. 18 is a schematic representation of an illustrative data storage of marker monitoring apparatus of FIG. 17.

FIG. 19 is a schematic representation of an illustrative data collection method for use with the training method of FIG. 1.

FIG. 20 is a schematic representation of an illustrative adaptive training apparatus for use with the training method of FIG. 1.

FIG. 21 is a schematic representation of an illustrative automated adaptive tutor for on-demand use of the training method of FIG. 1.

### DETAILED DESCRIPTION

In the following detailed description, reference is made to the accompanying drawings that form a part hereof, and in which are shown by way of illustration several specific embodiments. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

The term “installation” refers to the process of training an individual or individuals to learn a specific set of skills, except in instances where the particular context of the disclosure indicates otherwise. For example, after an individual experiences a training protocol, a skill set may be “installed” in the individuals. Preferably, the skill set is effectively installed; meaning that the individual can later recall or perform the skill set in a conscious or subconscious manner as part of training or in the real world. Installation of a skill set may refer to initial introduction, additional reinforcement of a skill set, or both. The terms “installation protocol” and “training protocol” may be used interchangeably throughout, except in instances where the particular context of the disclosure indicates otherwise.

The term “environment” refers to an element of time and space, which may be characterized by factors influencing the trainee's ability to successfully perform and achieve a desired outcome. An environment may also be described as a situation or a scenario. Environmental factors may include stressors, such as threats, obstacles, poor visibility, visual or aural distractions, and the like. Environmental factors may also include complicating factors, which may require decision making, such as multiple potential threats, multiple paths to a target destination, tactical choices for protecting lives, multiple strategies for achieving the desired outcome, and the like.

Furthermore, the term environment may include a “simulated” environment or a “real-world” environment. A “simulated” environment (e.g., simulation) is constructed by a trainer. In some examples, a “simulated” environment is designed to: introduce factors a trainee is likely to be exposed to in the real world, elicit similar cognitive and emotional effects that a trainee may experience in the real world, present artificial tasks that exercise skills likely to be used in the real world, or combinations thereof. In contrast, a “real-world” environment is an environment outside of training. A “real-world” environment typically has real-world consequences and may elicit cognitive and emotional effects that may be imitated in a simulated environment. A “real-world” environment may correspond to an actual event in the real world or a projection of likely events in which the trainee would perform. For example, a “simulated” environment may use simunitions (e.g., blanks) in a weapon to imitate live ammunition in “real-world” environment.

In general, the present disclosure relates to, among other things, installation protocols for improving performance of a skill or a skill set in stressful environments. In particular embodiments, the disclosure relates to a method of training to subconsciously install a sequence of skills under simulated stress designed to improve performance of the skills in stressful real-world environments, which integrates psychophysiological state management, stress inoculation, and cognitive skills training. The method may, in various embodiments, be described as an installation protocol for at least subconscious installation of skills, which includes several simulated environments to be introduced to a trainee in quick succession designed to install a set of critical skills in an accelerated manner that will be retained for use in sequence along a critical skill path. In many embodiments, the method includes an installation protocol designed to simultaneously install skills for situational awareness, stress resistance, critical decision-making, and programming motor-skills in an accelerated manner. While the present disclosure is not so limited, an appreciation of various aspects of the disclosure will be gained through a discussion of the examples provided below.

Much of this disclosure is directed to training individuals to successfully perform skills in complex, high-stress situations, particularly immediate threat-to-life situations wherein successful performance may protect and save lives. This method can be utilized for any number of trainees, types of trainee populations, and various trainee performance goals. It should be understood that the following discussion of the skills, simulated environments, tasks, and goals of this invention make reference to weapons training for immediate threat-to-life environments, and the like, generically, and that this discussion is not limiting to such an application, but that such aspects may be adapted to the specific outcomes, environments, and trainee population selected for training. It should also be understood that the method could be used with applications other than just weapons training, such as vehicular operation (e.g., aviation), manual security operations, safety classes, or any other scenario where performance of skills under stress is desired.

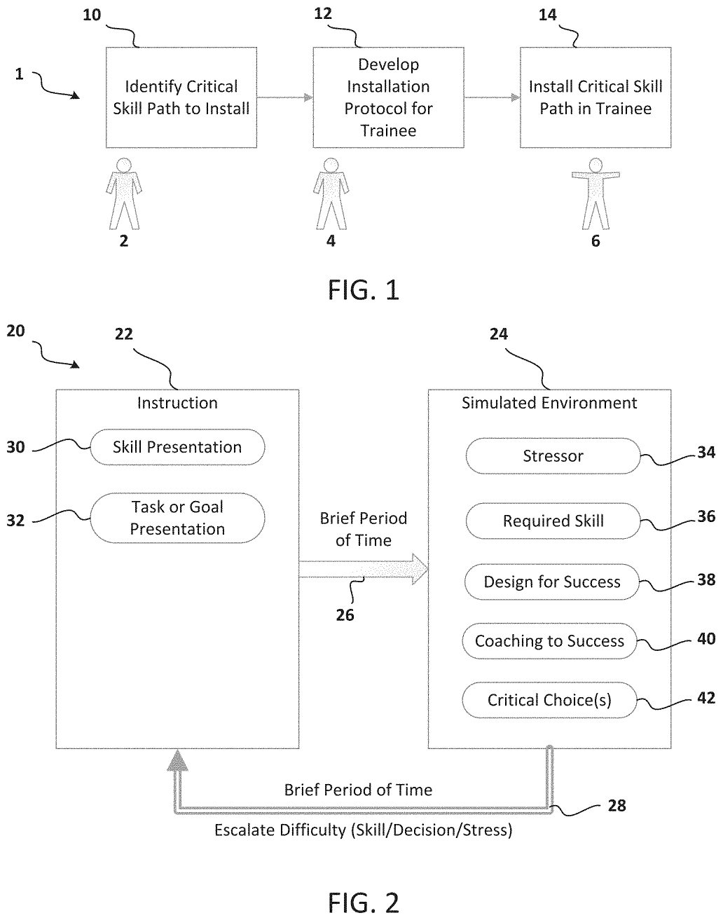

With reference first to FIG. 1, an illustrative training method 1 is shown as a schematic representation and includes a critical path process 10, a development process 12, and an installation process 14. In many embodiments, the training method 1 involves at least an expert 2, a trainer 4, and a trainee 6. For purposes of the present disclosure, a “trainee” may refer to an individual or more than one individual (e.g., a trainee population). In several illustrative embodiments, the trainee 6 is unaware of the details of the critical path process 10 and the development process 12. Furthermore, in various illustrative embodiments, the trainee 6 is unaware of parts of the installation process 14.

In some illustrative embodiments, one or more processes 10, 12, 14 of the training method 1 are automated. For example, in at least one embodiment, the trainer 4 can be described as including an automated intelligent tutor (preferably incorporating automated marker monitoring, automated adaptive training, and automated adaptive tutoring on-demand in real-time), as an alternative to or in addition to a human trainer, which can be configured to carry out some or all of the training method 1, such as the installation process 14, as described herein elsewhere.

The training method 1 may begin with understanding the desired outcome or performance for the trainee 6 in various real-world environments. In many illustrative embodiments, the training method 1 is designed to install one or more skills, such as vision skills, sensory cue acuity skills, state management skills, pattern recognition skills, and information processing and decision-making skills.

As used herein, “state management” refers to an ability to manage one's psychophysiological state. A psychophysiological state may include an emotional state, a physiological state, or a combination of both. A psychophysiological state may be influenced, for example, by stress. In general, the term “state management” may be used herein interchangeably with “stress management”, unless the particular context of usage dictates otherwise.

In an illustrative critical path process 10, a sequence of skills called a “critical skill path” is identified that, when employed by an individual, may provide a high probability for desirable outcomes in the various real-world environments identified. Although other skills may be helpful to achieving the desired outcome, the critical skill path may only include the skills necessary for success. In some cases, for example, certain skills may be helpful in only one environment but not others and may be deemed unnecessary to achieving the desired outcome for a selected set of environments.

In many embodiments, the critical skill process 10 involves eliciting a sequence of skills used by an expert 2. An expert 2 may be defined as an individual who has performed better than the average population in achieving desired outcomes in the various real-world environments. In certain embodiments, an expert 2 is an outlier who performs significantly better than average and sometimes beyond typical performance expectations in selected real-world environments. In some cases, there may only be a handful of experts for the selected real-world environments. One example of an expert 2 is a mission commander or shuttle pilot for a space program who has successfully completed a space flight mission, representing a pinnacle of training and proven skill under operational stress.

Certain techniques known in the art, such as surveying and interviewing, may be utilized to elicit psychophysiological states and performance levels of the expert 2 to ascertain a set of skills and their sequence. The sequence of skills may represent a critical skill path. For example, an expert 2 may look for a limited number of visual aspects to identify a potential threat, representing the critical skill path, to quickly identify a threat, whereas non-experts may study numerous less important aspects resulting in successful identification but with sub-optimal performance.

Eliciting these critical skill paths from experts 2 may form a basis for training others to perform to their individual potential. For example, the expert 2 may inherently possess superior abilities, which are not trainable in others. However, the cognitive strategy and motor skills that the expert 2 performs in a sequence to achieve optimal results represents a critical skill path that is trainable in others.

In at least some embodiments, skill sequences are elicited from more than one expert 2. In various embodiments, skill sequences are elicited from three or more experts. However, in other embodiments, skill sequences may be elicited from two experts. When multiple skill sequences are elicited from multiple experts, the critical skill path can be determined in response to an analysis of overlapping skills. For example, the set of skills utilized by all experts may be defined as the critical skills for success. Accordingly, the sequence of those critical skills may be defined as the critical skill path.

The critical path process 10 may be performed by the trainer 4. That is the trainer 4 may elicit the critical path from the expert 2. However, any skilled elicitor of motor skills, cognitive skills, and emotional states may perform the critical path process 10.

Once a critical skill path has been identified, an installation protocol may be developed for the trainee 6 in development process 12. In many illustrative embodiments, the installation protocol developed includes multiple, immersive-learning experiences that incorporate stress inoculation. For example, a trainee may be put into a dynamic environment involving identifying targets against which incapacitating force should be used from non-targets and making critical aggressive and defensive decisions. To install skills in the trainee, the experiences may be designed to integrate the presentation and use of one or more specific critical skills, such as expanding a field of view and a useful field of view, identifying changes in psychophysiological state of the self and others, regulating psychophysiological states in the self, making effective decisions under stress, and enhancing memory of the environment.

In general, the purpose of the installation protocol is to install the critical skill path in the trainee 6 so that the trainee may recall and perform the critical skill path in various environments, which may include complicating factors and stressors. In many embodiments, the defining characteristics of the trainee 6 are incorporated into developing an appropriate and effective installation protocol, for example, in terms of identifying the stress, skills, and arrangement thereof to be introduced through the installation protocol.

The trainee 6 may be defined by various characteristics, such as skill level, experience level, background, or other individual or collective characteristics. For example, a trainee population may comprise a group of teachers to be trained to respond appropriately to various lock-down scenarios and whose experiences in self-defense range from none to military veteran. In such cases, the installation protocol may be developed to account for the least experienced individual in terms of motor skills but also stress management skills.

The development process 12 may be performed by the trainer 4 or any skilled developer of installation protocols according to the present disclosure.

Several principles may be integrated into the development process 12 to influence the design of the each experience or the arrangement of experiences in the installation protocol. Non-limiting examples of principles that may be used include: providing an early anchoring experience in success under stress of a major psychophysiological barrier, initial installation of the critical skill path into the subconscious of the trainee bypassing the conscious learning process, introducing stress management skills experientially first without conscious learning, and introducing aspects of multiple critical skills in one experience.

One principle that may be used in the installation protocol is the early introduction of a foundational experience, upon which subsequent experiences are built to introduce new skills and/or reinforce the foundational experience. In many embodiments, the first experience in the training protocol preferably includes a basic stressor designed to elicit a foundational psychophysiological state in the trainee (e.g., a baseline stress response state). A basic stressor preferably elicits a large stress response in the trainee to experience a foundational psychophysiological state under which the trainee is likely to operate to successfully perform the critical skill path in a real-world environment. In at least some illustrative embodiments, the stress response corresponds to the greatest psychophysiological barrier to performing an essential critical skill. For example, an essential critical skill may be the motor skill of shooting another individual, and the greatest psychological barrier is using deadly force against another human being. In this manner, the trainee experiences success in one of the most important psychophysiological states for performing the critical skill path early in training.

It may be preferable to design an experience with sufficient fidelity to simulate a real-world environment. For example, a high-fidelity simulated environment for a gun battle may include live actors with real weapons and simunitions, the presence of visual obstructions, and an overlay of human sounds of panic before visual contact can be made. With sufficient fidelity, a simulated experience may be able to “trick” the trainee's cognitive processes into the desired psychophysiological state, which may be a severe stress response state in some cases.

After the foundational psychophysiological state is experienced, the training protocol may then continue on to introduce experiences that repeatedly elicit the foundational psychophysiological state to reinforce performance in the foundational psychophysiological state. Preferably, the continuing experiences also introduce new skills, reinforce skills, and/or add more stress in the context of the foundational psychophysiological state.

In several illustrative embodiments, the first experience is preferably designed to favor success to provide an anchoring experience in success for the trainee. The continuing experiences are also preferably designed to favor success to continue to build successful experiences into the foundational psychophysiological state. The trainee preferably experiences all critical skills in various appropriate levels of stress associated with the foundational psychophysiological state.

Another principle that may be used is the installation of competence into the subconscious of the trainee. In some other training methods, skills are taught according to a conscious model of competence. In other such methods, a trainee's stage of competence may begin with unconscious incompetence (cannot perform, does not know of skill), move to conscious incompetence (unsuccessfully performs known skill), then go to conscious competence (successfully performs skill with conscious focus), and ends with unconscious competence (successfully performs skill without conscious focus). In one example related to driving, a trainee may begin not knowing that there is a skill associated with driving, move to knowing about the skill but not performing it will, then go to driving well while focusing on each aspect, and end with driving well without consciously thinking about the various aspects of driving.

In contrast to the conscious model, illustrative embodiments of this disclosure relate to an installation protocol designed to skip the conscious steps during initial installation and instead install the skill subconsciously. In other words, the trainee preferably moves from unconscious incompetence directly to unconscious competence. This may facilitate subconscious recall of skills and speed of recall by bypassing conscious processes. Subconscious performance of skills may enable the trainee to react more quickly than conscious performance of skills (e.g., parallel processing versus linear processing).

One technique to facilitate subconscious installation, which may be incorporated into development, is providing instructions to perform actions to accomplish the skill so that the skill is experienced, rather than conscious explaining the skill before it is experienced. In some illustrative embodiments, after initial installation of a skill, the skill is consciously explained after one or more experiences to further refine the skill.

Another technique to facilitate subconscious installation, which may be incorporated into development, is removing conscious objections to the performance of a skill. For example, in order to shoot another individual, the trainee may experience a conscious moral objection. To remove the conscious objection before instructing the trainee to shoot, the trainer may first setup the experience by telling the trainee that the individual is an immediate life-threat to the trainee or another innocent individual and/or that the trainee has legal authority to eliminate the threat by deadly force. In this manner, the conscious moral objection is preferably removed so that the experience of shooting an individual and the installation of associated skills are taught subconsciously without conscious interference.

Yet another principle that may be used the integration of a stressor into each experience to elicit a psychophysiological state with each experience of learning a new skill to develop stress inoculation. For example, each experience is preferably designed to include a stressor to install a stress management skill and designed to instruct the use of at least one more motor skill or cognitive strategy skill. In many embodiments, the stress management skill is not explicitly taught to the trainee, rather the stress management skill is developed through instructions to perform an action. In other words, the trainee may not be told that one of the instructions in an experience represents is a stress management skill before or during the experience. In this manner, an individualized stress management skill associated with the stressor is preferably installed into the preconscious or subconscious of the trainee, rather than rising to a conscious level of learning.

The skill may be individualized, for example, because the trainee's own unique psychological platform has developed a subconscious pathway for achieving success while being exposed to the stressor. Because the installation is preferably subconscious, the trainee is also preferably not able to articulate or explain exactly why performing the set of instructions in an experience is successful at managing stress, or even be able to identify which instruction is associated with a stress management skill.

In various illustrative embodiments, after experiencing the stressor and associated effects of performing the instruction representing the stress management skill in at least one experience, the stress management skill may be refined at a conscious level by an explanation or articulation of the effects to the trainee. In some embodiments, conscious refinement may include teaching a stress recognition skill to the trainee to increase awareness of psychophysiological state. In many cases, a trainer preferably has more conscious awareness of stress management elements than a trainee before the installation protocol begins. After the installation protocol, the level of conscious awareness of the trainee may still be below the level of the trainer, for example, if the level of conscious awareness of the trainer is not necessary for the trainee to successfully perform. In other words, the conscious refinement of the stress management skill trainee may only be taught up to the necessary level for successful performance. Conscious refinement may be performed after a series of experiences, for example, at least three experiences to refine after developing a preconscious foundation for the stress management skill.

Further still, one principle that may be used is the development of accelerated learning instructions for each experience. For example, each experience may include instructions for the trainee to learn a new critical skill. The development process 12 may include identifying the critical aspects of the critical skill and developing instructions for the trainee to perform only the necessary aspects required to successfully execute the skill. In one example of teaching how to properly hold a fire arm, a trainee may be instructed to: clasp the hands together, point the index fingers toward a target, curl one index finger to create a space, and extend the index finger and leave the space open to grip a fire arm, and then, a trainer may place a fire arm into the grip to teach the trainee the critical aspects of holding a fire arm and to give a successful experience of properly holding a fire arm. In a similar process as described with respect to eliciting the critical skill path, the critical aspects of each critical skill may be elicited from an expert or multiple experts as part of the critical skill process 10.

In various illustrative embodiments, the instructions for one experience may initially install the critical aspects for multiple critical skills. In at least some illustrative embodiments, each experience concurrently installs aspects of at least three critical skills. In at least one illustrative embodiment, each experience concurrently installs aspects of three to five critical skills. Non-limiting examples of critical skills that may be taught in one experience include: a mental willingness to shoot another person, aiming a firearm, grip of the firearm, breathing, and positioning of the body. Such an experience, for example, may include the instructions to properly hold a fire arm plus instructions to: extend the arms, align the weapon sight and one eye, take a slow breath, and fire the weapon at an individual.

In some illustrative embodiments, one or more of these principles are used to develop the experiences in the training protocol. In at least one illustrative embodiment, all of these principles are used to develop the training protocol.

Once the installation protocol has been developed, the process of installing the critical skill path into the trainee 6 may be performed in installation process 14. In many illustrative embodiments, the trainee may be put into a stressful environment within a brief period of time after beginning the installation process 14.

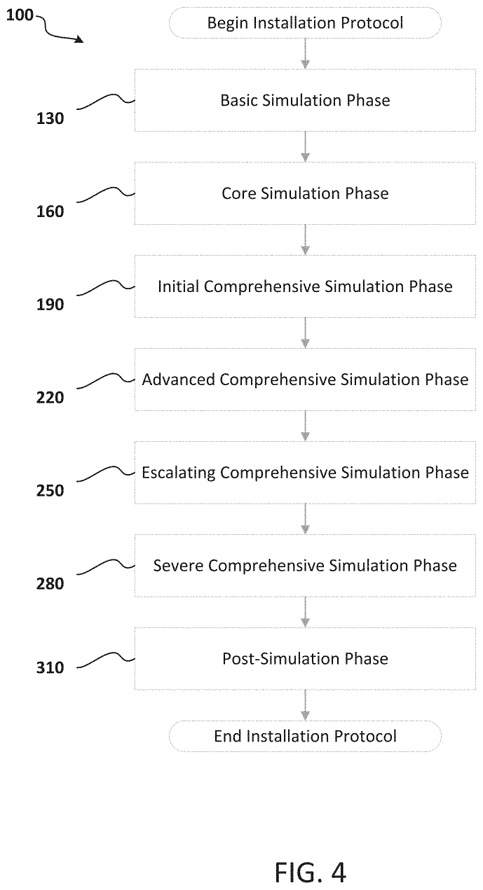

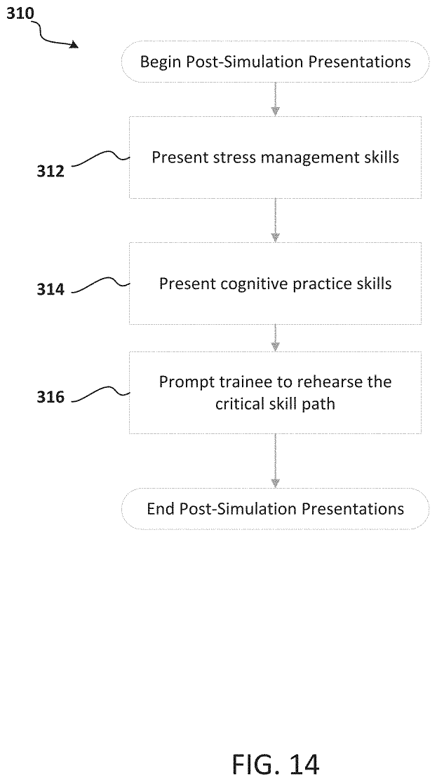

An illustrative installation process 14 may include one or more live sessions in which the trainee 6 may receive instruction, coaching, and exposure to environments in real-time, live, or in-person from the trainer 4. However, pre-recorded or interactive sessions are contemplated. In addition, the installation process 14 may include one or more practice sessions for the trainee 6 to practice learned skills outside of a session with the trainer 4. For example, the practice sessions may involve mental rehearsal by the trainee 6 after cognitive practice skills are taught by the trainer 4 (e.g., process 310 of FIGS. 4 and 14).

In many embodiments, the live sessions are designed to introduce all critical skills in the critical skill path in a series of experiences. In such embodiments, the practice sessions may then be designed to improve retention of the critical skill path so that installation may be considered “permanent”, or at least more likely to be retained over a period of time, such as a year, a few years, five years, ten years, or more.

To install the critical skill path, live sessions may occur within a limited duration. In some embodiments, live sessions of the installation process 14 occur within one week. In various embodiments, live sessions occur within a span of a few days. In further embodiments, live sessions occur all in one day. In at least one embodiment, live sessions take place in only a few hours. However, additional live sessions beyond the limited duration are also contemplated, for example, as a refresher course or for installation of additional critical skills.

To install the critical skill path, practice sessions may occur beyond the limited duration of the live session, prompted by the trainer 4, for example, at the end of the live sessions. In many embodiments, practice sessions by the trainee 6 may occur after about 7 days, after about 14 days, and after about 21 days from the end of the live sessions. Although additional practice sessions may not be necessary to successful retention, additional practice sessions are also contemplated.

With reference now to both FIGS. 1 and 2, the training method 1 may include developing a set of experiences 20 designed to install the critical path in the trainee 6. An illustrative series of experiences 20 is shown in FIG. 2 as a set including an instruction 22 and a simulated environment 24. The illustrative experiences 20 may be part of a live session, for example, in which the trainer 4 presents an instruction 22 and introduces a simulated environment 24 after a brief period of time 26. In a brief period of time 28 after the simulated environment 24 is completed, a next set may begin with presenting a next instruction 22 and then introducing a next simulated environment 24 after a next brief period of time 26 and so forth.

In various illustrative embodiments, the trainee 6 is consciously unaware of some critical skills along the critical skill path, even after the critical skill path has been installed. Also, the trainee 6 may be consciously unaware of all of the critical skills being taught in an experience 20 (e.g., may be aware of one motor skill out of 3 motor skills and a stress management skill) or aware of the psychophysiological state to be elicited by the experience 20 as designed.

In many embodiments, the next set escalates the difficulty in some manner relative to the previous set. In at least some embodiments, the complexity or stress-level of the simulated environment 24 is increased. Examples of increasing complexity may include requiring the use of an additional skill or require making of an additional decision (e.g., adding a decision tree or adding another decision layer).

In several illustrative embodiments, the installation process 14 includes sets of experiences 20 that begin at a level of difficulty appropriate for the characteristics of the trainee 6. Each subsequent experience 20 increases in difficulty in some manner throughout the installation process 14, at least until the level of difficulty is commensurate with a potential real-world environment. In some cases, the difficulty is increased until severe stress is elicited in the trainee 6. The difficulty may also be increased until multiple levels of decision-making trees are required to successfully complete the simulated environment 24.

In some illustrative embodiments, the instruction 22 may include a skill presentation 30 and a task or goal presentation 32 for the trainee 6 to perform or achieve in the simulated environment 24. The skill presentation 30 may incorporate concepts from accelerated learning. A complicated skill may be broken down into fundamental components and only one component is presented 30 at a time for installation. For example, the skill of handling a weapon may be broken down, and the basic motor skill of aligning an eye to the weapon may be presented in skill presentation 30. In this manner, accelerated learning techniques may facilitate brief skill presentations 30 that take only a few minutes—or even a few seconds.

The skill may be described as being taught experientially. In other words, the instruction 22 may be presented and, within a brief period of time 26, the skill must be used to successfully perform a task or achieve a goal in the simulated environment 24 according to the task or goal 32. In this manner, learning the skill is embedded in the context of the simulated environment 24, which is designed to install the skill at a subconscious level of processing in the trainee 6.

In many embodiments, “a brief period of time” may be defined as a period of time in which the trainee 6 does not have the opportunity to consciously process the experience 20. In some illustrative embodiments, a brief period of time 26, 28 is less than about 10 minutes. In various illustrative embodiments, a brief period of time 26, 28 is less than about 5 minutes. In at least some embodiments, a brief period of time 26, 28 is less than about 3 minutes. In some embodiments, a brief period of time 26, 28 is as little time is practical within the training protocol. Generally, a brief period of time 26, 28 will be more than one second. Although a “brief period of time” is defined above specifically with respect to FIG. 2, this definition applies to the use of a “brief period of time” throughout this disclosure.

Once the trainee 6 is introduced into the simulated environment 24, the trainee 4 must complete the task or goal presented 32. In general, a simulated environment 24 may be described as an environment including a staging area with active and inactive parts. For example, a person holding a weapon may be an active part of the simulated environment 24, representing a threat. Furthermore, that person may be dressed as a combatant or as a civilian.

In many embodiments, each simulated environment 24 may be designed differently, for example, to experientially teach a different skill presented 30 or to experientially test the skill presented 30 under different factors. The design of a simulated environment 24 may be described as including any number of design factors, such as a stressor 34, the use of a required skill 36 (e.g., presented in the skill presentation 30), a design for success 38, coaching to success 40, a critical choice 42. In some embodiments, each simulated environment 24 includes at least a stressor 34 and a required skill 36. Design of the simulated environment 24 to incorporate these factors may occur during a development stage (e.g., process 12) of the installation protocol.

In many illustrative embodiments, the stressor 34 is designed to elicit one or more stress response states in the trainee 6. The stressor 34 may be selected to simulate or relate to a real-world stressor that the trainee 6 is likely to encounter. Alternatively, or in addition, the stressor 34 may be an artificial construction selected to exercise the use of a required skill 36 out of the context of a likely real-world environment (e.g., asking the trainee to recite a social security number backwards while under fire, which preferably requires the use of a stress management skill).

The stress response state elicited in the trainee 6 may be unique to each individual. By successfully using the required skill 36 to complete the task or goal 32 in the simulated environment 24 in the stress response state due to exposure to the stressor 34, the trainee 6 gains an experience anchored in success associated with the stressor 34 and the use of the required skill 36. The trainee 6 may then be further inoculated against the stressor 34 in a similar environment, which may facilitate performance of the required skill 36. Stress inoculation may be further reinforced through recognition of the stress response state in the trainee 6 by the trainee 6.

Typically, the trainer 4 refrains from providing instruction or commands while the trainee 6 is in a simulated environment 24, which may improve unconscious installation and the fidelity of the simulated environment (e.g., similarity to a real-world environment). In order to facilitate gaining successful anchoring experiences by the trainee 6, the simulated environments 24 may incorporate a design for success 38 (e.g., a design to favor success) for the trainee 6 without trainer 4 assistance.

Design for success may be defined as setting the level of difficulty of a simulated environment 24 to the experience and skills of the trainee 6 such that the simulated environment 24 may be completed by the trainee 6 without more than a brief instruction 22. In some cases, a trainee 6 may be veterans with combat experience having with robust stress resilience. Accordingly, appropriate simulated environments 24 incorporating a design for success 38 may include a familiar stressor 34, such as the immediate threat of a fully-armed soldier, in the early phases of the installation process 14 that would otherwise compromise successful performance in other trainees 6 (e.g., cadets with no combat experience) at a similarly early phase. In other words, typically the design for success 38 depends upon characteristics of the trainee 6. By way another example, a hand gun novice trainee 6 may be instructed to discharge their weapon and hit a living target at a distance of 5 feet or less under stressor 34. Due to the close proximity of the target, the likelihood of a successful outcome (hitting the target) can be maximized.

In the installation process 14, some simulated environments 24 may not incorporate a design for success 38. In many embodiments, a simulated environment 24 to install a critical skill is designed to allow for failure only after previous simulated environments 24 have already anchored the critical skill in success. For example, in a later phase of the installation process 14, a simulated environment 24 may be designed to allow the trainee 6 to fail in the simulated environment 24 (e.g., not achieve the goal) using a critical skill that was already used to successfully perform a task or goal in a previous simulated environment 24. Typically, the trainee 6 has the opportunity to examine why the failure occurred for use in subsequent simulated environments 24.

Although the trainer 4 typically does not need to instruct the trainee 6 in a simulated environment 24, the trainer 4 may incorporate coaching to success 40 in the simulated environment 24 when a coaching need is observed. The coaching may be done in real-time while the simulated environment 24 is active (e.g., before successful completion or a task or goal). In some embodiments, the trainer 4 may prompt the trainee to utilize a skill. The prompted skill may be a previously presented skill. However, the trainer 4 may also present the skill during the simulated environment 24. In this manner, the skill can also be described as being taught experientially. In at least one embodiment, the trainer 4 may observe a stress response state in the trainee 6 and prompt the trainee 6 to utilize a stress management skill. In many cases, the trainer 4 may be considered a skilled observer for identifying response states in the trainee 6. The prompt may include a skill presentation 30 of a new stress management skill during the simulated environment. For example, the trainer 4 may walk the trainee 6 through basic breathing control to facilitate the completion of the task or goal presented 32. In this manner, the simulated environment 24 may incorporate coaching to success 40.

The simulated environment 24 may also incorporate a critical choice 42 designed to require the trainee 6 to make a critical decision. The difficulty of a simulated environment 24 may be proportional to the number of critical choices 42 included. In many embodiments, a critical skill may facilitate making a critical decision by the trainee 6, such as a cognitive strategy skill. For example, a critical cognitive strategy skill may include deciding whether to identify a person as a threat by looking at certain factors.

As a critical skill is installed the trainee 6 under stress, cognitive and neurological changes may manifest as one or more observable markers. A marker may be described as external (e.g., a human-observable cue) or internal (e.g., a sensor-observable a biomarker). A marker may be correlated with the critical skill through observation and analysis of the subconscious installation of the critical skill.

In various illustrative embodiments, a trainer has experience or training to identify external signs of a psychophysiological state or change thereof, as well as concurrent, successful performance of a skill. Such a trainer may observe and then associate a particular marker observed with the critical skill being experientially learned. Non-limiting examples of external markers include: a change in skin coloration of the trainee, respiration characteristics, posture, muscle tension, pupil dilation, and behavioral responses. By correlating markers to performance outcomes, the trainer may infer the marker to represent the learning or performance of the specific critical skill, being in a particular psychophysiological state (e.g., stress response state), changing a psychophysiological state in the trainee 6, or a combination thereof. Additional observations may lead to statistical confirmation of such relationships.

The trainee may exhibit various markers associated with learning a critical skill (e.g., particular eye or gross motor movements, skin color, respiratory patterns). However, the term “critical markers” may be described as the measurable markers essential to differentiating between a successful and unsuccessful subconscious installation under stress. In many cases, the critical marker preferably indicates an optimal psychophysiological state associated with successful performance of the skill under stress. The presence of the critical marker may be generalized to an optimal psychophysiological state for learning similar skills under stress, as well, in some cases. In various illustrative embodiments, the critical marker may also represent the change in psychophysiological state before/during/after the skill is installed or performed under stress.

The observation of markers may facilitate the development of a database of experiences to enhance current or subsequent installations. For example, the trainee 6 may be coached to success in stress management or other skills in real-time by making adjustments to ensure certain markers are observed while installing the skill. In another example, a subsequent trainee may benefit from an altered simulation design to further elicit the same marker or to elicit a different marker while performing the skill. Patterns and relationships between markers, skills, psychophysiological states, and trainee characteristics can also begin to be inferred with enough experiential observations and analysis, for example. For example, a sequence of critical markers may be developed and associated with the sequence of skills in the critical skill path preferably representing the optical psychophysiological states of learning the critical skill path.

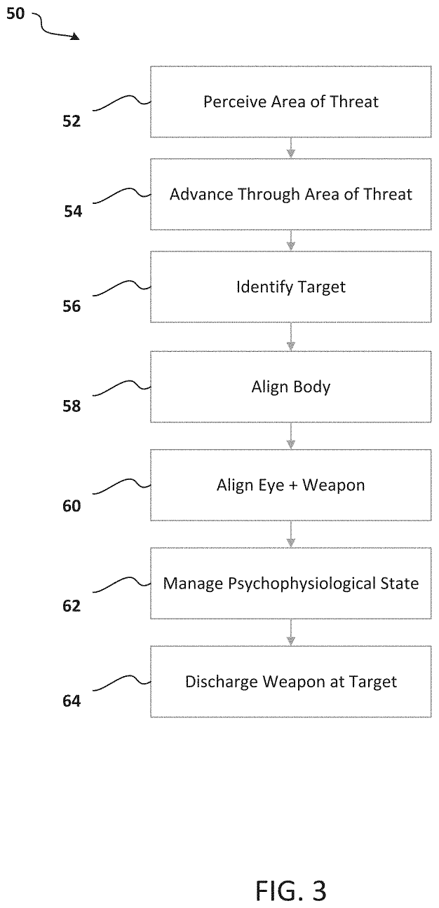

Turning now to FIG. 3, an illustrative critical skill path 50 is shown that may be installed. In the illustrated embodiment, the critical skill path 50 includes multiple critical skills arranged along the path. Non-limiting types of critical skills include a motor skill, a stress management skill, a cognitive strategy skill, and a stress recognition skill.

The illustrative critical skill path 50 begins with the skill of perceiving an area of threat 52 and ends with discharging a weapon at a target 64. In particular, this critical skill path 50 may be applicable in environments wherein an individual must move through a dangerous area including potential threats or targets.

Perceiving an area of threat 52 may be described as a cognitive strategy skill used in assessing the environment. After performing skill 52, the critical skill path 50 continues to advancing through the area of threat 54, which may be described as a cognitive strategy skill in deciding where to move in a safe manner. A motor skill may also be associated with advancing through an area of threat 54, such as a technique for holding a weapon while moving.

Then, a target may be identified 56, which may include a cognitive strategy skill of identifying characteristics of a threat or target versus another person. Once a target has been identified, the body may be aligned 58 toward the target, which may be described as a motor skill. More specifically, aligning the body 58 may be a supporting skill for the following motor skill of aligning the individual's eye and weapon 60 to the target. Such alignment may be influenced by the alignment of the body, for example.

After the weapon has been properly aligned, the next critical skill may involve managing the psychophysiological state 62 of the individual, which may be described as a stress management skill. The skill may further involve a stress recognition skill to notice the individual's psychophysiological state, and a cognitive skill of whether to use a stress management skill to change the individual's current psychophysiological state. As a simple example, the psychophysiological state may be too stressed or too not stressed enough, prompting the individual to make a change with a stress management skill.

The critical skill path 50 ends with discharging a weapon at the target 64. In particular, by accessing the critical skill path 50, which may be installed according to an installation protocol, the individual has taken the steps that an expert would take in a similar environment, while managing individual stress response states. In this manner, the illustrative critical skill path 50 may facilitate successful performance in the environment for non-expert individuals.

A critical path may be installed according to the illustrative installation protocol 100 as shown in FIG. 4. The installation protocol 100 may be described as experiences to install the critical skill path in a series of phases. In particular, the illustrative installation protocol 100 shows phases of the live sessions involving a trainer and at least one trainee. In at least some illustrative embodiments, the installation protocol 100 includes a basic simulation phase 130, a core simulation phase 160, an initial comprehensive simulation phase 190, an advanced comprehensive simulation phase 220, an escalating comprehensive simulation phase 250, a severe comprehensive simulation phase 280, and a post-simulation phase 310. In many illustrative embodiments, each of the phases is separated only by a brief period of time, which may facilitate subconscious installation of the critical skill path.

Some simulations, or simulated environments, may be designed to favor success throughout the installation protocol 100. In many illustrative embodiments, the simulations in a first set of phases are designed to favor success of the trainee, such as phases 130, 160, 190. In at least some illustrative embodiments, the simulations in a second set of phases are not designed to favor success of the trainee, such as phases 220, 250, and 280. Although some simulations are not designed to favor success, the simulations may not be designed to favor failure either, but rather to exercise the presented skills to accelerate learning in the trainee or to increase stress, for example.

As used herein, a “comprehensive simulation” or “comprehensive simulated environment” may refer to a simulation in which all previously presented skills are designed to be required to complete the simulation. In several illustrative embodiments, a comprehensive simulation preferably requires all previously presented critical skills to be used to complete a simulation. For examples, comprehensive simulations in phases 190, 220, 250, and 280 may require all critical skills previously presented in the installation protocol 100 to be used to complete the comprehensive simulation. However, not all critical skills may be used in every comprehensive simulation. For example, in some embodiments, due to critical decisions made, not all motor skills in the critical skill path may be required to be used in a comprehensive simulation.

In the illustrative basic simulation phase 130, a trainee experiences a stressful environment and coached to success. For example, in training weapon usage, the basic simulation phase 130 may involve the task of shooting at another person. Specifically, within the first ten minutes of the live session, the trainee is presented basic motor skills (e.g., weapon handling) and tasked with discharging a simunitions-equipped weapon at another armed and armored individual. The basic stressor in this experience is the action of shooting at another individual with simulated lethal force. During the experience, a basic stress management skill may be presented in real-time by a trainer and coached to manage stress by the trainer. Herein throughout, the term “trainer” may be used to refer to one individual trainer, a group of trainers, or any other appropriate facilitator of the installation protocol 100.

After completing the task, the stress management skill may be reinforced by an experiential debrief of the stressful environment. Additional stress management skills may also be presented and experientially taught. Stress management skills presented in the basic simulation phase 130 may include breathing, relaxation, enhancing visual perception, among others.

Each trainee may experience a psychophysiological state (e.g., stress response state) in response to the basic stressor that is unique to each individual. Preferably, the psychophysiological state is a baseline stress response state. A stress recognition skill may be presented during or after completing the task as part of the coaching. The stress recognition skill may teach the trainee to notice psychophysiological cues that indicate the psychophysiological state that the individual experienced when completing the task. The trainer may be a skilled observer with the ability to recognize and to describe such cues to aid in teaching the stress recognition skill. The stress recognition skill may also enable the trainee to observe and to recognize cues in others, as well. Non-limiting examples of cues indicating psychophysiological state include: a change in skin coloration of the trainee, respiration characteristics, posture, muscle tension, and pupil dilation.

The experience of the basic simulation phase 130 may be designed with a stressor that is likely to elicit a similar psychophysiological state as a likely real-world stressor. By successfully completing the basic simulation integrating the basic stressor, the trainee has anchored the psychophysiological state to success. The psychophysiological state may then be reinforced throughout the installation protocol 100 in subsequent experiences, which may improve inoculation to the basic stressor.

After the trainee has successfully completed the basic task and established an experience anchored in success with the stressor, the installation protocol 100 may continue with the core simulation phase 160. In the core simulation phase 160, the critical skills of the critical skill path are experientially taught to the trainee. In many embodiments, each critical skill involves a motor skill. The sequence of critical skills along the critical skill path may also include cognitive strategy skills, for example, relating to deciding whether to use a particular motor skill or a stress management skill. Because the critical skill path is derived from an expert, and often multiple experts, the individual is preferably taught to problem solve in the same way that an expert problem solves while also learning related motor skills and stress management skills in the context of the stress under which the trainee is likely to face in real-world environments.

In several illustrative embodiments, the difficulty of each experience in the core simulation phase 160 is increased. The number of critical skills, the stress level, or the number of critical decisions may increase the level of difficulty for each experience. For example, the first simulated environment may require the use of one critical skill under a first stressor to successfully complete a first task. Then, the second simulated environment may require the use of two critical skills (including a cognitive strategy skill to make a critical decision) under a second stressor (more stressful than the first stressor) to successfully complete a second task. The third simulated environment may require the use of three critical skills (including one or more cognitive strategy skills to make two critical decisions) under a third stressor (more stressful than the second stressor) to successfully complete a third task. Such a pattern of increasing the difficulty of one or more components (critical skills, stress, or critical decisions) may continue until all critical skills have been used to complete tasks in the core simulation phase 160.

During the core simulation phase 160, the increasing stressors may be ramped such that the trainee is likely to become overwhelmed before the end of the phase. Although the trainee's inoculation to stress is important to the installation protocol 100, stress coaching may also be utilized between experiences to further teach and install stress management skills. In many illustrative embodiments, stress management skills are only presented in the core simulation phase 160 after three or more experiences of escalating stressors. However, other intervals are also contemplated, depending on observations by the trainer or other skilled observer.

The critical skills may be described as being divided into essential critical skills and supporting critical skills. The essential critical skills may be considered the most important critical skills. In many illustrative embodiments, the critical skill path involves one essential critical skill and multiple supporting critical skills related to the essential critical skill. For example, in weapons training, the essential critical skill may involve the motor skill of aligning the eye, the weapon, and the target in an efficient manner.

In at least some embodiments, the essential critical skill is the first critical skill presented and designed to be required to complete the first task. Then, supporting critical skills, such as consistently aligning the body to the target, are presented afterward.

In at least one embodiment, the essential critical skill is preferably required to be used to complete each task in the core simulation phase 160, for example, to reinforce performance and retention of the essential critical skill. Similarly, in at least one illustrative embodiment, each subsequent stressor may integrate the basic stressor to further reinforce inoculation to the basic stressor. For example, the basic stressor of shooting at another individual with simulated lethal force may be incorporated into the first stressor as shooting at another individual with simulated lethal force while under simulated cross-fire.

Some of the critical skills may be presented in an order of importance simulation-by-simulation, which may different than the order of the critical skill path. Within each simulated environment, however, the critical skills are preferably used in the order of the critical skill path whether the simulated environment is designed to require the use of all critical skills or only a subset thereof. For example, an essential critical skill may be taught before a related supporting skill, even though the supporting skill is used earlier in the critical skill path. In one specific example, to complete the task of hitting a target with the weapon, a simulated environment may be designed to require the trainee to align the body to the target (supporting skill) before aligning the eye and the weapon to the target (essential skill) to successfully hit the target, even though the alignment of the eye and weapon skill was already taught in an earlier simulation.

After initial critical skills have been presented and used to complete respective tasks (e.g., in basic stimulation phase 130 and core simulation phase 160), a trainee may be subjected to an initial comprehensive simulation phase 190. In many illustrative embodiments, other skills and advanced components of critical skills have not been presented to the trainee. In various embodiments, the initial comprehensive simulation phase 190 comprises one experience. In the experience, the trainee may be instructed to achieve a goal and be introduced into an initial comprehensive simulated environment requiring the use of all critical skills in the critical skill path.

In some embodiments, a simulated environment of the initial comprehensive simulation phase 190 is designed to favor success. The trainer may also coach the trainee to success by prompting the use of various critical skills. The use of all critical skills together to successfully achieve a goal under stress anchors a successful experience in the trainee, which may be the first successful anchoring experience utilizing all critical skills in the critical skill path. In contrast, although the core simulation phase 160 may require the use of an essential critical skill in all simulated environments, those simulated environments preferably do not include one experience designed to incorporate the use of all critical skills in the critical skill path.

Furthermore, the difficulty introduced in the initial comprehensive simulation phase 190 may be designed to be even greater than the core simulation phase 160. In particular, a simulated environment in the initial comprehensive simulation phase 190 may be designed to elicit a greater stress response state than any simulated environment in the core simulation phase 160. For example, in weapons training, the initial comprehensive simulation phase 190 may involve engaging multiple attackers or multiple groups of hostages. Further, saving each group of hostages may require different actions along the critical skill path, for example, dictated by the critical choices presented.

Following the initial comprehensive simulation 190, the advanced comprehensive simulation phase 220 may begin. The advanced comprehensive simulation phase 220 may include an advanced series of end-goal instructions and an advanced series of comprehensive simulated environments. In many illustrative embodiments, advanced components of skills may be presented and used to achieve desired outcomes over the advanced series. In at least some embodiments, an instruction and a simulated environment in this phase 220 may be separated only by a brief period of time.

An advanced component may relate to a critical skill, for example, as an advanced aspect of a motor skill, a cognitive strategy skill, or a stress management skill. In other words, the critical skill may include one or more advanced components. The advanced component may be described as an evolution (e.g., refinement) or a specialization (e.g., useful for specific situations). In one example, an advanced component of a motor skill may be an empty hand combat skill while maintaining control of a weapon before discharge. To teach and test the empty hand combat skill, a comprehensive simulated environment may require the trainee to be engaged at close range and to use the empty hand combat skills to grapple with multiple potential targets before being discharging the weapon.

This advanced component may be described as being taught in context (e.g., experientially), as well as being taught concurrently with other skills. In one example, the trainee may be required to grapple before weapon discharge, and also may be required to use an advanced multiple-level cognitive strategy component to make critical decisions about which threats are targets, and may be further required to use an advanced stress management component to manage a psychophysiological state while being exposed to multiple stressors.

In some illustrative embodiments, a comprehensive simulated environment in this phase 220 may be designed to be more difficult than a previous comprehensive simulated environment in this phase 220, such as an immediately preceding comprehensive simulated environment. In some embodiments, difficulty may be increased by requiring the trainee to use a different combination of skills (e.g., a new combination), exposing the trainee to a stressor designed to elicit a more stressful response state, or requiring the trainee to make more critical decisions. Preferably, the simulation is designed to require that the combination of skills be used in the order of the critical skill path by the trainee to successfully achieve the desired outcome. In various illustrative embodiments, a comprehensive simulated environment in this phase 220 may be designed to favor successful achievement of the desired outcome. In at least some embodiments, all advanced components along the critical skill path are designed to be required in the advance comprehensive simulation phase 220.

An escalating comprehensive simulation phase 250 may follow the advanced comprehensive simulation phase 220. The escalating comprehensive simulation phase 250 may include an escalating series of end-goal instructions and an escalating series of comprehensive simulated environments. In many illustrative embodiments, the escalating comprehensive simulation phase 250 may be designed to maintain and escalate the stress response state in the trainee over the escalating series. In at least some embodiments, an instruction and a simulated environment in this phase 250 may be separated only by a brief period of time, which may facilitate maintenance of the stress response state.

A comprehensive simulated environment in this phase 250 may escalate the stress response state by requiring the trainee to use a different combination of skills (e.g., a new combination), exposing the trainee to a stressor designed to elicit a more stressful response state, or requiring the trainee to make more critical decisions. Accordingly, in this phase 250, a comprehensive simulated environment may be more stressful than a previous comprehensive simulated environment, such as an immediately preceding comprehensive simulated environment.

In many illustrative embodiments, a comprehensive simulated environment in the escalating series may require the trainee to use a combination of previously presented skills and to make one or more critical decisions to successfully achieve a desired outcome. Typically, the combination of skills may be required to be used in an order according to the critical skill path to successfully achieve a desired outcome. In some embodiments, the trainer may prompt the trainee after introduction into a comprehensive simulated environment in the escalating series to use one or more previously presented skills to facilitate successful achievement of a desired outcome. In various embodiments, all previously presented skills may be required to be used in the escalating comprehensive simulation phase 250.

Following the escalating comprehensive simulation phase 250, the next phase may be a severe comprehensive simulation phase 280. The severe comprehensive simulation phase 280 may include a severe series of end-goal instructions and a severe series of comprehensive simulated environments. In many illustrative embodiments, the severe comprehensive simulation phase 280 may be designed to elicit severe stress while testing the previously presented skills in the installation protocol 100 over the severe series.

A comprehensive simulated environment in this phase 280 may elicit a greater stress response state in the trainee than any previously elicited stress response state (e.g., in other phases). In some embodiments, the phase 280 may be designed to elicit a greater stress response state in even an experienced trainee than a similar real-world experience (e.g., designed to simulate more stress than real experiences of a veteran police officer with twenty years of experience). In one example, a severe comprehensive simulation phase 280 for training active shooter skills may require the trainee to enter a building with a team while under fire, passing screaming civilians (e.g., children as role players), and advancing toward a shooting threat in a closed room (e.g., classroom) before discharging the weapon at the shooting threat.

In at least some embodiments, an instruction and a simulated environment in this phase 280 may be separated only by a brief period of time, which may facilitate the severe stress response state. A comprehensive simulated environment in the severe series may be different from another comprehensive simulated environment in the severe series by requiring the trainee to use a different combination of skills, exposing the trainee to a different stressor designed to elicit a severe stress response state, or requiring the trainee to make a different critical decision. In some embodiments, all previously presented skills may be required to be used in the severe comprehensive simulation phase 280.

In at least some embodiments, a comprehensive simulated environment in the severe series is not designed to facilitate success. As such, the trainer may observe the trainee fail in a simulated environment. In some illustrative embodiments, within a brief period of time after identifying a failure of the trainee to achieve a desired outcome, the trainer may prompt the trainee to identify one or more previously presented skills that would have facilitated successful achievement of the desired outcome. Within a brief period of time after identifying such skills, the trainee may be presented with another end-goal instruction and introduced into another comprehensive simulated environment in the severe series.

A post-simulation phase 310 may follow the severe comprehensive simulation phase 280. The post-simulation phase 310 may be the last phase of the installation protocol 100. In many cases, after the severe comprehensive simulation phase 280, the trainee is in a severe stress response state, which may be difficult to manage. The post-simulation phase 310 may present one or more stress management skill designed to reduce the effects of the severe stress response state, which may be presented within a brief period of time after the severe comprehensive simulation phase 280. A particular stress management skill may be effective for a particular stress response state.

In many illustrative embodiments, the stress management skills may be presented in an order according to the designed sequence of stress response states to be elicited in each comprehensive simulated environment in the severe series. The stress management skill may be presented in the context of the stressor from each comprehensive simulated environment. In this manner, the stress management skills are taught experientially in an effective order for use in similar real-world environments.

Furthermore, the post-simulation phase 310 may include a presentation of one or more cognitive practice skills to practice previously presented skills. Cognitive practice skills may include an active visualization skill, a mental rehearsal skill, an active stress management skill, a replay skill, or a recall skill. In many embodiments, the trainee may be prompted to use the cognitive practice skills to practice the previously presented skills in one or more sequences, for example, according to a previously introduced simulated environment. The trainer may prompt the trainee to practice at regular intervals, such as one week, two weeks, four weeks, or more. Such practice may be done by the trainee without the presence of the trainer by utilizing the cognitive practice skills presented.

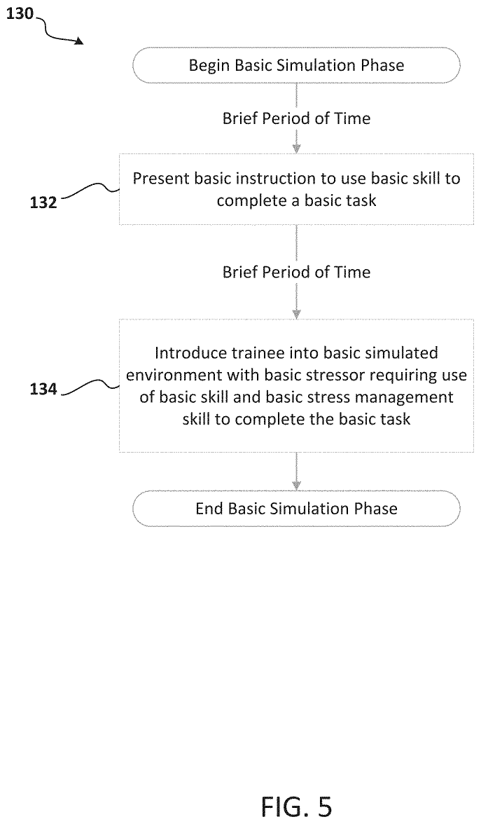

With the general aspects of phases in the installation protocol 100 described, various examples of phases and components are described herein in more detail beginning with FIG. 5, which shows an illustrative basic simulation phase 130 as a flowchart including various steps. The phase 130 may begin with a basic instruction is presented in step 132 to use a basic skill to complete a basic task. The basic skill may be related to the critical skill path. For example, the basic skill may be a critical skill or an aspect of a critical skill. After another brief period of time, the trainee is introduced into a basic simulated environment with a basic stressor in step 134, which may require the trainee to use the basic skill and a basic stress management skill to complete the basic task. The basic stress management skill may be presented experientially in a conscious or pre-conscious/subconscious manner, for example. Once the trainee has completed the basic task, the trainee has gained an experience anchored in success with overcoming a basic stressor, and the basic simulation phase 130 ends.

In the present disclosure, the stress management skills referred to preferably increase the stress inoculation capability of the trainee and are preferably developed by experiential presentation. An experiential presentation may present the stress management skill in a conscious manner or a preconscious/subconscious manner. In some cases, the stress management skill may be presented by the trainer as a conscious step for the trainee to do during the simulation. For example, the trainee may take the conscious step of controlling breathing as instructed by the trainer. In other cases, the stress management skill may be developed by introducing the trainee to a simulation in which the trainee subconsciously develops the stress management skill by completing the simulation.

Preferably, a simulation is designed to encourage the subconscious development of the stress management skill. For example, a simulation that is designed to require the trainee to shoot at another live human being may subconsciously install a stress management skill into the preconscious processing of the trainee that overcomes the stress response state elicited (e.g., a major psychophysiological barrier) in a manner that is unique to the trainee's neurological and cognitive traits. As a result, the trainee preferably begins inoculation of the stress response state elicited by the stressor (e.g., being required to shoot at another live human being) at a subconscious level. Once installed subconsciously, the basic stress management skill may be consciously refined afterward, for example, by presenting a stress recognition skill.

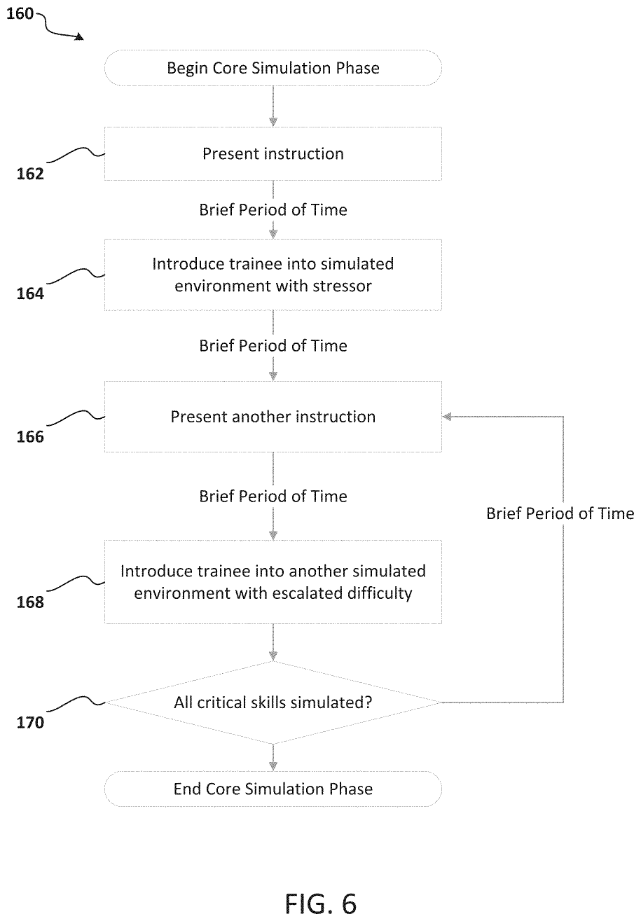

An illustrative core simulation phase 160 is shown in FIG. 6 as a flowchart including various steps. The phase 160 begins with presenting an instruction in step 162 (e.g., core instruction), which may direct the trainee to complete a task (e.g., core task) using a first critical skill. After a brief period of time, the trainee is introduced into a simulated environment (e.g., core simulated environment) with a stressor in step 164, which preferably requires the trainee to use the first critical skill to complete the task. The first critical skill may be presented experientially, either during instruction or during the simulation. The stressor may also be greater than the basic stressor (e.g., designed to elicit a greater stress response state). The first critical skill may be an essential critical skill.

After a brief period of time, another instruction is presented in step 166, which may direct the trainee to complete a task using a second critical skill. After a brief period of time, the trainee is introduced into a simulated environment with a stressor in step 168, which preferably requires the trainee to use the second critical skill to complete the task. In many embodiments, the trainee is also preferably required to use the first critical skill to complete the task. The second critical skill may be presented experientially, either during instruction or during the simulation. The stressor may also be greater than the previous stressor of step 164. The second critical skill may be a supporting critical skill.

According to step 170, each of steps 166 and 168 may be repeated until all critical skills have been used in simulated environments. In other words, all critical skills along the critical skill path are presented and used in simulated environments before the core simulation phase 160 ends. In many embodiments, each simulated environment in step 168 is more difficult (e.g., stressful) than an immediately preceding simulated environment. In several embodiments, a simulated environment preferably requires all previously presented critical skills to be used to complete the task before the end of core simulation phase 160.

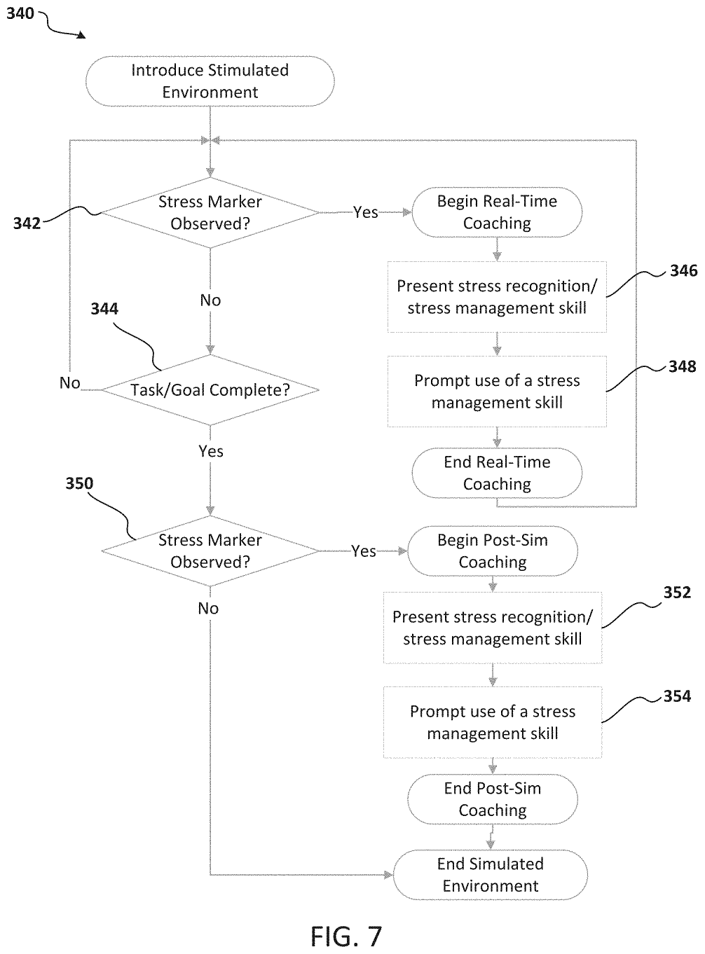

During some phases, such as the core simulation phase 160, a stress coaching process 340 may be performed by the trainer after the trainee is introduced into a simulated environment as shown in FIG. 7. Beginning with step 342, the trainer may observe a stress marker in the trainee. If a stress marker is observed, real-time coaching may begin with presenting a stress recognition skill or a stress management skill in step 346 (e.g., coaching to success). For example, if a relevant stress recognition skill or stress management skill has not yet been presented, then the trainer may choose to present it. Then, using a stress management skill, such as the basic stress management skill presented during the basic simulation phase 130, may be prompted in step 348.

A similar process to the stress coaching process 340 may also be used for real-time coaching to prompt the use of a critical motor skill or a critical cognitive strategy skill instead of a stress-related skill. In such a process, the trainer may observe a stress marker or other indication that the trainee is not using an appropriate skill to successfully complete the simulation.

If a stress marker is not observed at first, the trainer may continue to observe the trainee until the task or goal is completed in step 344. Once the task is completed, the trainer can also observe the trainee for a stress marker in step 350. If a stress marker is observed, post-simulation coaching may begin with presenting a stress recognition skill or a stress management skill in step 352. This stress recognition or management skill may be the same as presented in step 346 (only if one was presented in real-time coaching) or may be a new skill not previously presented to the trainee. The trainer may also prompt the use of a stress management skill in step 354 to end post-simulation coaching. At this point, the simulated environment may be considered completed or finished.

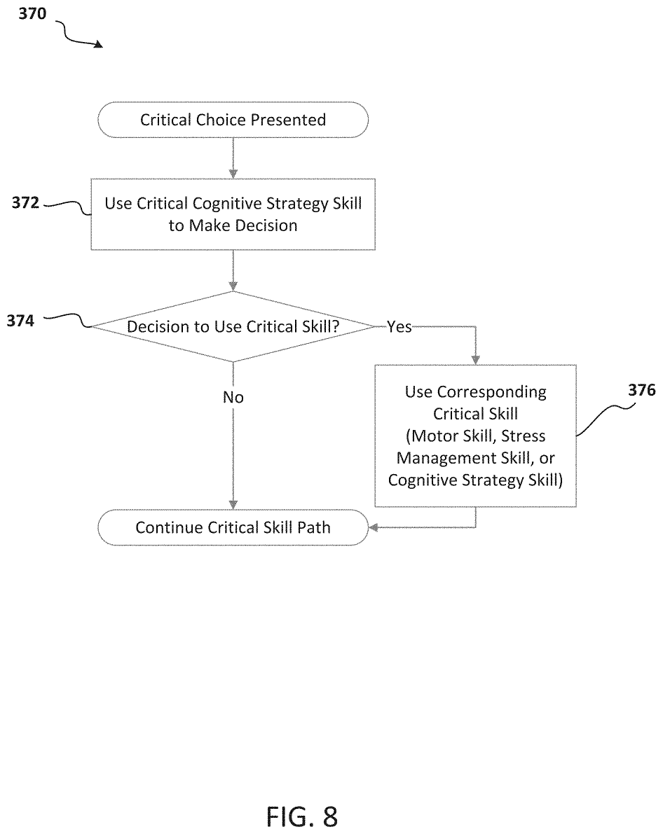

An illustrative critical decision process 370 as shown in FIG. 8 is one example of using a cognitive strategy skill to make a critical decision. In a simulated environment, a critical choice may be presented for which the trainee may use a critical cognitive strategy skill to make a decision (e.g., a critical decision) in step 372. In the illustrated example, the critical decision may be related to whether to use another critical skill in step 374. If so, the trainee may use the corresponding critical skill in step 376, which may be a motor skill, a stress management skill, or even a cognitive strategy skill. In some embodiments, the cognitive strategy skill corresponds to only a motor skill or a stress management skill. The critical skill path is continued once either the corresponding critical skill is used in step 376 or the trainee decides not to use the critical skill in step 374.

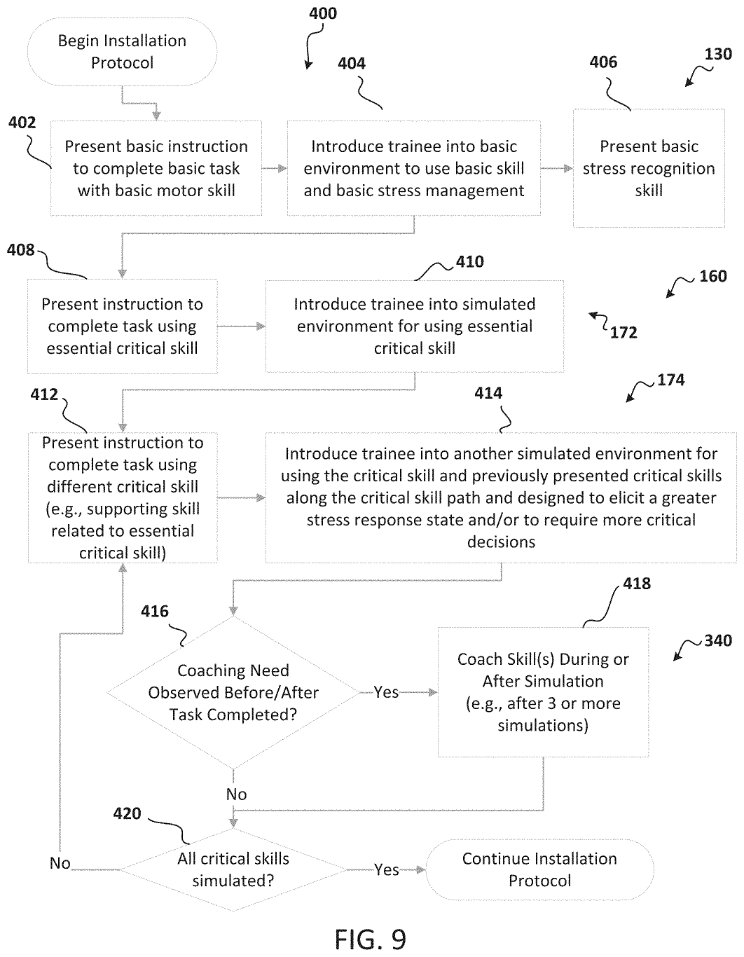

Having described phases for presenting new critical skills of the critical skill path to a trainee, an illustrative skill introduction process 400 as shown in FIG. 9 incorporates a basic simulation phase 130, a core simulation phase 160 including an essential critical skill introduction process 172 and at least one supporting skill introduction process 174, and a coaching stress process 340. Although not shown explicitly in FIG. 9, many of the steps are separated only by a brief period of time (e.g., a short time) as described herein elsewhere in more detail.

The illustrative skill introduction process 400 may begin an installation protocol with an illustrative basic simulation phase 130. For example, the process 400 may begin with presenting a basic instruction to complete a basic task using a basic motor skill is presented in step 402, followed by an instruction of the trainee into a basic simulated environment requiring use of the basic skill and a basic stress management skill in step 404. Once the basic stress management skill has been experienced, a basic stress recognition skill may be presented in step 406 to consciously refine the basic stress management skill.

The process 400 may continue with an illustrative core simulation phase 160. For example, shortly after the trainee has finished the basic simulated environment in step 406, an illustrative essential critical skill introduction process 172 may begin with an instruction to use an essential critical skill may be presented as the first critical skill in step 408. The trainee is then introduced into a simulated environment requiring use of the essential critical skill 410.

Once the essential critical skill 410 has been used, the illustrative core simulation phase may continue with an illustrative supporting skill introduction process 174 beginning with presenting an instruction to complete a task use a supporting critical skill related to the essential critical skill in step 412. Shortly after, the trainee is introduced into another simulated environment requiring the use of a different critical skill (e.g., supporting critical skill) and the essential critical skill and designed to elicit a greater stress response state than the simulated environment of step 414.

Before or after the task is completed (e.g., before or after finishing the simulation), the trainer may observe to see whether coaching is needed in step 416. If not, the process 400 iterates (e.g., continues to repeat) the series of presenting instructions to use critical skill in step 412 and introducing the trainee into simulated environments in step 414 until all critical skills of phase 160 have been simulated in step 420 and the installation protocol can continue. In each iteration of the steps 412,414, the use of all previously presented critical skills in addition to the critical skill presented may or may not be required to complete the task.

Various iterations may also include a stress coaching process 340 with the corresponding steps 416, 418. For example, if a coaching need is observed in step 416, the trainer may coach the trainee during or after the simulation in step 418. In some cases, the trainer may observe a need in real-time before the task is completed and decide to coach the trainee before the task is completed. In other cases, the trainer may observe the need before the task is completed but choose to wait until the task is completed before coaching.

In many illustrative embodiments, the coaching does not include presenting a new stress management skill until three or more critical skill simulated environments (e.g., step 410 and two iterations of step 414) have been finished. For example, the trainee may only have experienced the basic stress management skill, which may be insufficient to cope with the cumulative stress effects of the first three simulated environments. In this manner, the stress level in the trainee is built up over at least three simulated environments before a new stress management skill would be presented to manage the built-up stress. After coaching, the process 400 continues to repeat the additional critical skill steps 412, 414 (and the coaching steps 416, 418 as needed) until all critical skill have been simulated in step 420.

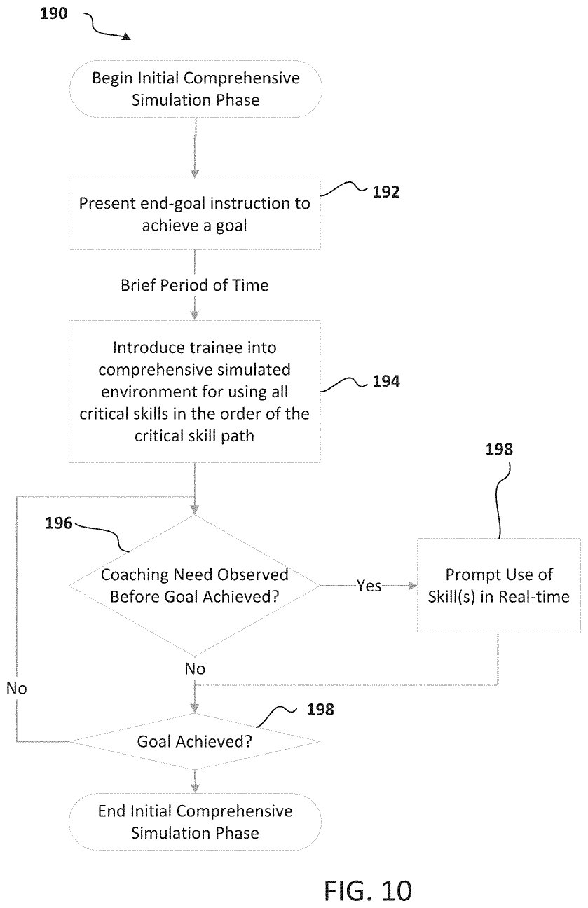

An illustrative initial comprehensive simulation phase 190 as shown in FIG. 10 is one example of an experience to test all previously presented critical skills. The initial comprehensive simulation phase 190 begins with presenting an end-goal instruction to achieve a goal in step 192. In contrast to an instruction to complete a task, an end-goal instruction to achieve a goal may not specify which critical skill is (or skills are) or tasks are required to achieve the goal. Within a brief period of time, the trainee may be introduced into a comprehensive simulated environment requiring all critical skills to be used to achieve the goal in step 194. In particular, the critical skills may be required to be used in the order of the critical skill path.

A coaching need may be observed before the goal is achieved by the trainee in step 196. If a coaching need is observed, the trainer may facilitate the use of skills in real-time in step 198 (e.g., coach to success). For example, because all critical skills in the order of the critical skill path may be required, the trainer may prompt the trainee to use the next critical skill. The trainer may continue to observe for a coaching need until the trainee achieves the goal in step 198 and the initial comprehensive simulation phase ends 190.

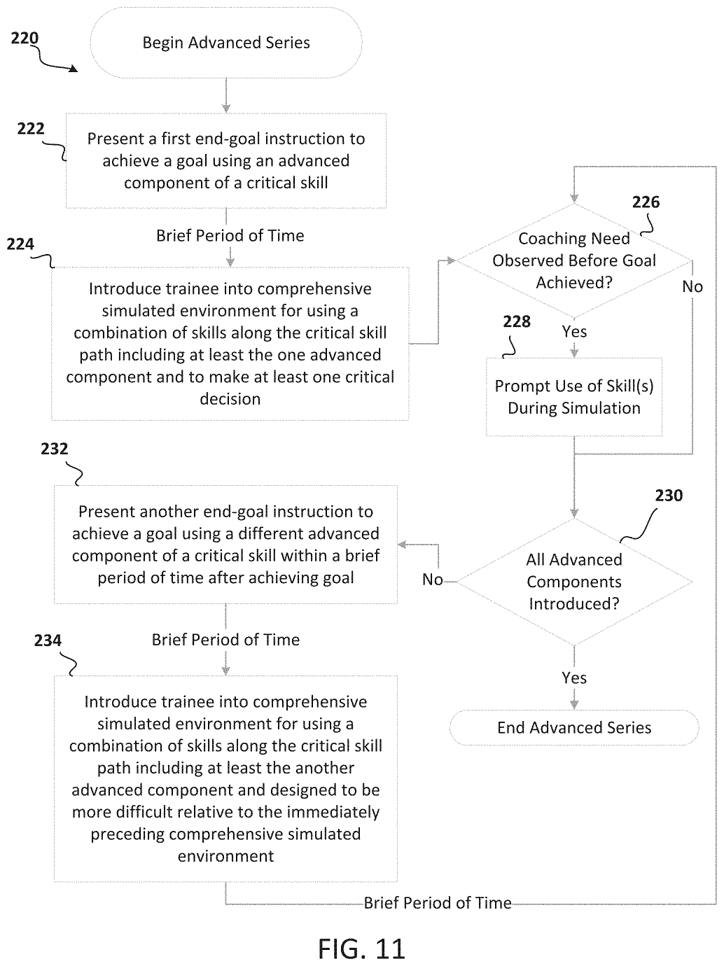

An illustrative advanced comprehensive simulation phase 220 as shown in FIG. 11 is one example of an advanced series of experiences to teach advanced components of critical skills. The advanced comprehensive simulation phase 220 begins with presenting a first end-goal instruction to achieve a goal using an advanced component of a critical skill in step 222. Within a brief period of time, the trainee is introduced into a comprehensive simulated environment in step 224 requiring a combination of critical skills including at least the advanced component presented to successfully achieve the goal. At least one critical decision may also be required to successfully achieve the goal in step 224.

Coaching may also be performed when a coaching need is observed before the goal is achieved in step 226. If a coaching need is observed, the trainer may prompt the use of a skill before the goal is achieved in the simulated environment in step 228.

After the goal is achieved in the simulated environment, the process 220 iteratively continues within a brief period of time to present another end-goal instruction to achieve a goal using a different advanced component of a critical skill in step 232 followed within a brief period of time by introducing the trainee into a comprehensive simulated environment requiring the use of the different advanced component of the critical skill in step 234.

Coaching may be included with any of the iterations. The iterations may continue until all advanced components have been introduced according to step 230 and the advanced series ends. In step 234, the simulated environment may also be designed to be more difficult relative to the immediately preceding comprehensive simulated environment.

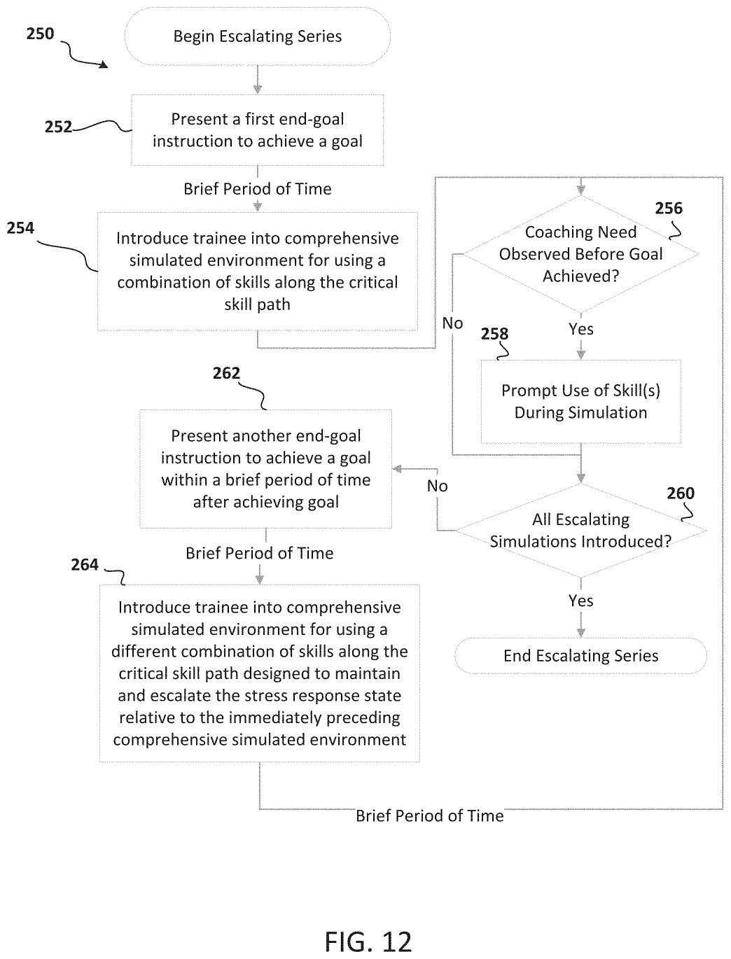

An illustrative escalating comprehensive simulation phase 250 is shown in FIG. 12 as one example of an escalating series of experiences to test critical skills along the critical skill path while maintaining and escalating stress. The escalating comprehensive simulation 250 begins with presenting a first end-goal instruction to achieve a goal in step 252. Within a brief period of time, the trainee is introduced into a comprehensive simulated environment requiring the use of a combination of skills along a critical skill path to successfully achieve the goal in step 254.

Coaching may also be performed when a coaching need is observed before the goal is achieved in step 256. If a coaching need is observed, the trainer may prompt the use of a skill before the goal is achieved in the simulated environment in step 258.

After the goal is achieved in the simulated environment, the process 250 iteratively continues within a brief period of time to present another end-goal instruction to achieve a goal in step 262 followed within a brief period of time by introducing the trainee into a comprehensive simulated environment requiring the use of a different combination of skills designed to maintain and escalation the stress response state of the trainee relative to the immediately preceding comprehensive simulated environment in step 264. Coaching may be included with each iteration. The iterations may continue until all escalating simulations have been introduced according to step 260 and the escalating series ends.

The escalating comprehensive simulation phase 250 may precede a severe comprehensive simulation phase 280. In some embodiments, the severe comprehensive simulation phase 280 may be considered part of or an extension of the escalating comprehensive simulation phase 250. In other words, a series of comprehensive simulations may maintain and escalate stress to culminate in a severe series of comprehensive simulations.

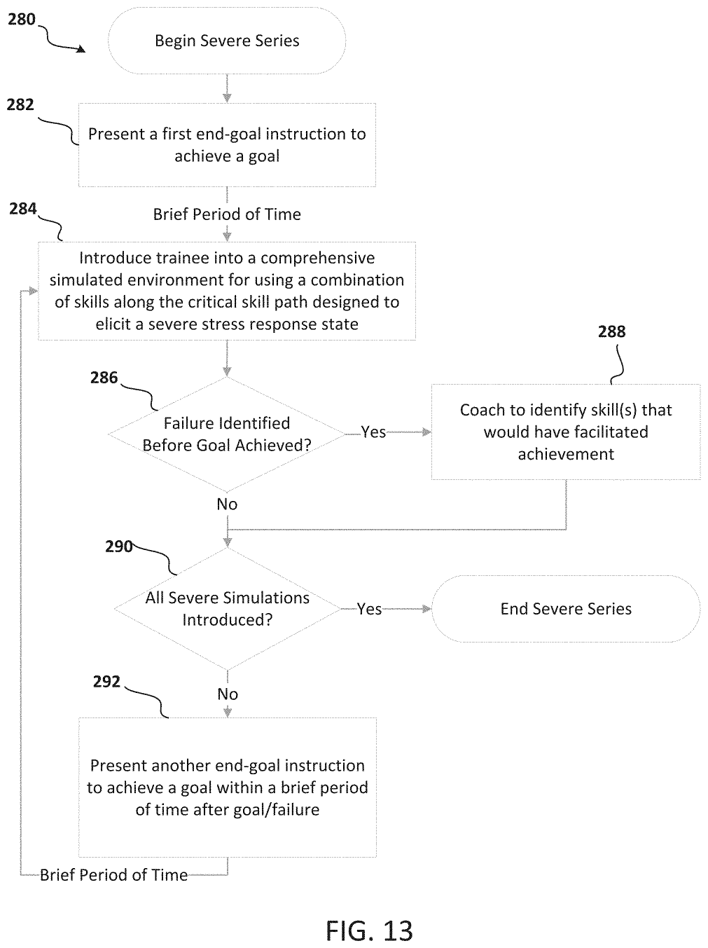

An illustrative severe comprehensive simulation phase 280 is shown in FIG. 13 as one example of a severe series of experiences to test critical skills along the critical skill path, including multiple levels of critical decision making, at severe levels of stress. The severe comprehensive simulation 280 begins with presenting a first end-goal instruction to achieve a goal in step 282. Within a brief period of time, the trainee is introduced into a comprehensive simulated environment requiring the use of a combination of skills along a critical skill path to successfully achieve the goal that is designed to elicit a severe stress response state in the trainee in step 284.

In various embodiments, the severe stress response state may be greater than any previous stress response state elicited in previous phases. In at least some illustrative embodiments, multiple critical decisions are preferably required to successfully achieve the goal.

In many illustrative embodiments, a comprehensive simulated environment in this phase 280 may not be designed to favor success. In other words, the trainee may be allowed to fail. Accordingly, before the goal is achieved, the trainer may observe the trainee to identify a failure. If a failure is identified before the goal is achieved, according to step 286, the trainer may coach the trainee. As shown, the trainer may coach the trainee to identify skills that would have facilitated achievement in the comprehensive simulated environment in step 288. One example of a technique to identify skills is to prompt walk the trainee to “walk backwards” in the scenario from the point of failure and recognize what went wrong and when. To “walk backwards” in the scenario, the trainer may guide the trainee to mentally recall the last steps taken, to physically retrace the last steps taken, or both. The trainer may then coach the trainee to consciously examine the last steps or to attempt the last steps again. This may facilitate the trainee's ability to discern steps to achieve a successful outcome. After this process, the comprehensive simulated environment may, in some embodiments, be considered finished.

After the goal is achieved or the simulated environment is finished, the process 280 iteratively continues within a brief period of time to present another end-goal instruction to achieve a goal in step 292 and to introduce the trainee into a comprehensive environment designed to elicit a severe stress response state 284 until all severe simulations have been introduced in step 290 and the severe series ends.

Each comprehensive simulated environment in the severe series may be different. In many illustrative embodiments, each comprehensive simulated environment may require a different combination of skills along the critical skill path to successfully achieve the goal. In at least some illustrative embodiments, the trainee is exposed to a different stressor also designed to elicit a severe stress response state. In at least one illustrative embodiment, the trainee is required to make different critical decisions.

The phases of simulated environments may be finished after the severe comprehensive simulation phase 280. In these phases, the brief periods between experiences coupled with the continual exposure to stressors and the immediate utilization of new critical skills and combinations may facilitate preconscious processing and installation of the critical skill path, which may translate into dramatically improved performance in complex, high-stress environments and long-term retention of the critical skill path.

Installation in the manner described may leave the trainee in an overwhelmed stress response state. After completing of the final phase 280, the trainee may be able to use previously presented stress management skills. However, additional stress management skills may also be presented in a post-simulation phase.

An illustrative post-simulation presentation phase 310 is shown in FIG. 14 as one example of a series of presentations to complete the live session portion of training, to transition the trainee to practice sessions without a trainer, and to complete installation of the critical skill path. The post-simulation presentation phase 310 may include a presentation of stress management skills along the critical skill path in step 312, a presentation of cognitive practice skills in step 314, and a prompt for the trainee to rehearse the critical skill path in step 316 to complete the post-simulation presentations.

Stress management skills presented in step 312 may be new stress management skills or previous stress management skills to be reinforced. Specific stress management skills may also be defined as real-time stress management skills or post-environment stress management skills. Post-environment stress management skills may be suitable for use outside of a stressful environment to manage stress. In many cases, a trainee will use stress managements skills after the severe series to ramp down from a severe stress response state. In some cases, a trainee may use stress management skills before entering, or shortly after entering, an environment in which the trainee must execute the critical skill path to either ramp down or ramp up. For example, the trainee may need to ramp up to attain the psychophysiological state identified in the basic simulation phase of training associated with successful performance.

In many embodiments, the real-time stress management skills are presented in the order of the critical skill path. In prior phases, the simulated environments preferably required use of critical skills in a pre-determined order along the critical skill path and, accordingly, were designed using stressors to elicit various stress response states likely to be encountered in a real-world environment in the same pre-determined order. In at least some illustrative embodiments, the stress management skill presented in step 312 are presented in the order of the critical skill path to install the stress management skills along the critical skill path. The stress management skills may be presented experientially in the context of likely stressors. For example, stressors may be revisited from previous simulated environments.

Cognitive practice skills presented in step 314 may include visualization and mental rehearsal skills. The use of visualization and mental rehearsal skills may be useful, for example, to reinforce the psychophysiological state anchored to successful performance, to facilitate remediation of recall errors during replay of events, and to reinforce performance of the critical skill path in complex, high-stress environments in the short-term and long-term.

With steps taken to install the critical skill path, to promote stress management for accessing the critical path, and to rehearse the critical path in complex, high-stress environments, the trainee may be prompted to rehearse the critical skill path at intervals during practice sessions in step 316 before ending the live session. Practice sessions following a live session may further cement and install the critical skill path associated with the psychophysiological states experienced during the installation protocol, particularly for long-term retention. In various embodiments, the practice sessions end after one month of the live session. In at least some embodiments, the intervals are regularly spaced, such as once a week. In at least one embodiment, the trainee is prompted to rehearse once a week for three weeks, thereby completing installation in a one-month timeframe from the beginning of training (e.g., the live session).

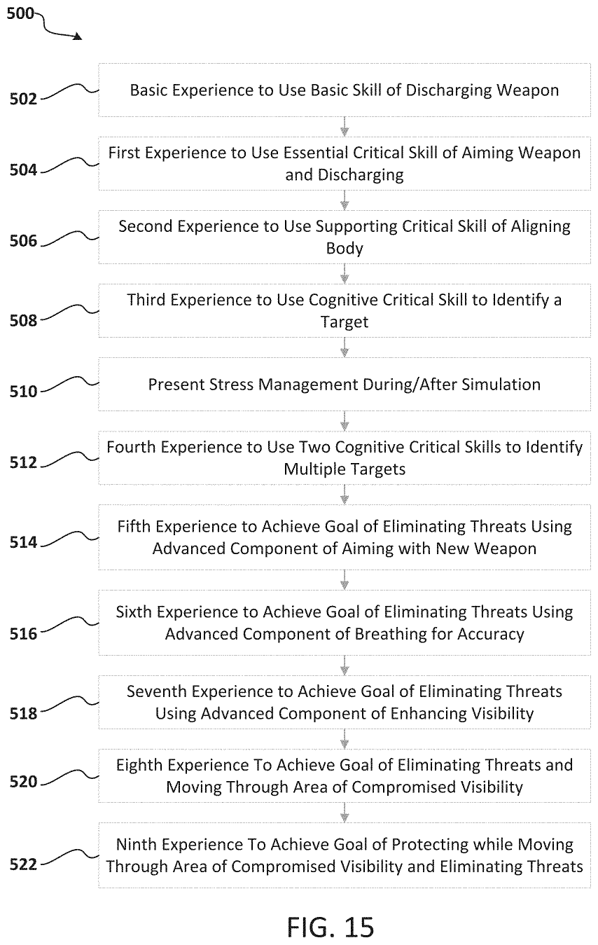

With the various phases of at least one illustrative training protocol being described, FIG. 15 shows at least one illustrative embodiment of a sequence of experiences 500 to install a critical skill path similar to critical skill path 50 (FIG. 3). In basic experience 502, a basic instruction to use a basic skill is presented, which may be discharging a weapon at a live human being. In certain embodiments, potential conscious moral objections may be removed by explaining the steps of the critical path presumed to have taken place leading up to discharging the weapon (e.g., determining a real threat). Briefly afterward, the trainee is introduced into a basic simulated environment in which the trainee must discharge a simunition equipped weapon at a human being at close range. Discharging the weapon at a human being may be considered a basic stressor. The close range of the basic simulated environment and/or the removal of conscious objections may be described as design factors that preferably facilitate success.

In first experience 504, which may be briefly after experience 502, a first instruction is presented to use an essential critical skill, which may be aiming a weapon and discharging at a live human being. Briefly afterward, the trainee is introduced into a first simulated environment in which the trainee must align one eye, the weapon, and a short-range target (e.g., real human being) and then discharge the weapon. The basic stressor in experience 502, which was discharging the weapon at a human being, is reinforced at this stage while introducing a new skill. The difficulty may also be increased, for example, by requiring aiming at a short-range target (farther than a close range target).

Then, in second experience 506, which may be briefly after experience 504, a second instruction to use a supporting critical skill is presented, which may be aligning the body while aiming. The alignment of the body as a supporting critical skill may or may not be instructed to be used before the essential critical skill. Briefly afterward, the trainee is introduced into a second simulated environment in which the trainee must align the body, then align one eye, the weapon, and a mid-range target, and finally discharge the weapon. The difficulty, or stress, in this experience 506 may be increased by the required use of a new skill in addition to previously presented skills and/or the challenge of aiming at a mid-range target instead of a close range or short-range target, for example.

Next, in third experience 508, which may be briefly after experience 506, a third instruction is presented to use a cognitive critical skill of identifying targets from potential targets. For example, the trainee may be instructed to look for certain weapons a potential target is holding, posture, facial expressions, etc. Briefly afterward, the trainee is introduced into a third simulated environment in which the trainee must decide to discharge the weapon at one or more potential targets, then upon deciding to discharge, align the body, align one eye, the weapon, and the mid-range target, and finally discharge the weapon. The difficulty, or stress, in this experience 508 may be increased by the required use of a new skill in addition to previously presented skills, for example.

Further, in coaching experience 510, a stress management skill may be presented during or briefly after experience 508. In many embodiments, after the third experience 508, the trainee is preferably in a severe stress response state that the stress management skill presented may help to manage. Briefly after presenting the stress management skill, the trainee may be required, for example, to use the stress management skill of a controlled breathing technique to manage the severe stress response state.

Continuing on, in fourth experience 512, which may be briefly after experience 510, a fourth instruction to use two cognitive critical skills is presented, which may be deciding to discharge at two potential targets. In other words, another critical decision tree may be added to the third experience 508. Briefly afterward, the trainee is introduced into a fourth simulated environment in which the trainee must decide to discharge the weapon at multiple potential targets, then upon deciding to discharge at each target, align the body, align one eye, the weapon, and the long-range target, and finally discharge the weapon. The difficulty, or stress, in this experience 512 may be increased by the required use of a new skill in addition to previously presented skills and/or the challenge of aiming at a long-range target instead of a mid-range target, for example. The fourth experience 512 may be described as including an initial comprehensive simulated environment, requiring the use of all initial critical skills previously presented.

In fifth experience 514, which may be briefly after experience 512, a fifth instruction is presented to achieve a goal of eliminating threats using an advanced component of aiming with a new weapon having an unknown alignment (e.g., sight location). Briefly afterward, the trainee is introduced into a fifth simulated environment in which the trainee must decide to discharge the weapon at multiple potential targets and the proper alignment for aiming the weapon, then upon deciding to discharge at each target, align the body, align one eye, the weapon, and the long-range target, and finally discharge the weapon. The difficult, or stress, in this experience 514 may be increased by the required use of a new skill component with a previously presented skill, for example.

Then, in sixth experience 516, which may be briefly after experience 514, a sixth instruction is presented to achieve a goal of eliminating threats using an advanced component of a breathing technique to improve accuracy. Briefly afterward, the trainee is introduced into a sixth simulated environment in which the trainee must decide to discharge the weapon at multiple potential targets, then upon deciding to discharge at each target, align the body, align one eye, the weapon, and the long-range target while compensating for breathing effects, and finally discharge the weapon. The difficult, or stress, in this experience 514 may be increased by the required use of a new skill component with a previously presented skill, for example.

Next, in seventh experience 518, which may be briefly after experience 516, a seventh instruction is presented to achieve a goal of eliminating threats using an advanced component of enhancing visibility to improve identification of a target in compromised visibility. Briefly afterward, the trainee is introduced into a seventh simulated environment in which the trainee must decide to discharge the weapon at multiple potential targets through smoke (compromising visibility of potential targets), then upon deciding to discharge at each target, align the body, align one eye, the weapon, and the target while compensating for breathing effects, and finally discharge the weapon. The difficult, or stress, in this experience 514 may be increased by the required use of a new skill component in a specific, challenging circumstance, for example.

Further, in eighth experience 520, which may be briefly after experience 518, an eighth instruction is presented to achieve a goal of eliminating threats under the stress of moving in an area of compromised visibility. Briefly afterward, the trainee is introduced into an eighth simulated environment in which the trainee must move through the area of smoke and must decide to discharge the weapon at multiple potential targets through smoke, then upon deciding to discharge at each target align the body, align one eye, the weapon, and the target while compensating for breathing effects, and finally discharge the weapon. The eighth experience 520 may be considered an escalated simulated environment, as the level of stress maintained and increased relative to the seventh experience 518. For example, the eighth experience 520 includes all skills required in the seventh experience 518 but further requires the trainee to move through a dangerous area. Also, in some embodiments, the trainee may or may not be told which critical skills are required to be used to achieve the goal.

Finally, in ninth experience 522, which may be briefly after experience 520, a ninth instruction is presented to achieve a goal of protecting another human being while moving through the area of compromised visibility and eliminating threats. Briefly afterward, the trainee is introduced into an ninth simulated environment in which the trainee must move through the area of smoke with objects obstructing lines of sight, identify a child to protect, carry the child to a safe location through an area of threat, and decide to discharge the weapon at multiple potential target through smoke. Then, upon deciding to discharge at each target align the body, the trainee must align one eye, the weapon, and the variable range target while deciding whether to compensate for breathing effects, and finally discharge the weapon. The ninth experience 522 may be considered a severe simulated environment, as the level of stress is designed to be severe (e.g., even more than a real-world environment). For example, the ninth experience 522 includes all skills required in the eighth experience 520 but requires more goals to be achieved, decisions to make, and challenges to overcome. Also, the trainee may or may not be told which critical skills are required to be used to achieve the goals.

In many illustrative embodiments, the installation method may be designed to ensure that at least a threshold level of stress has been experienced by the trainee. This may facilitate the installation of skills, especially the subconscious installation of skills. In at least some embodiments, the design preferably ensures that at least the threshold level of stress is experienced while learning each of the critical skills of the critical skill path. In at least one illustrative embodiment, the design preferably ensures that at least the threshold level of stress is experienced before installing a critical skill of the critical skill path.

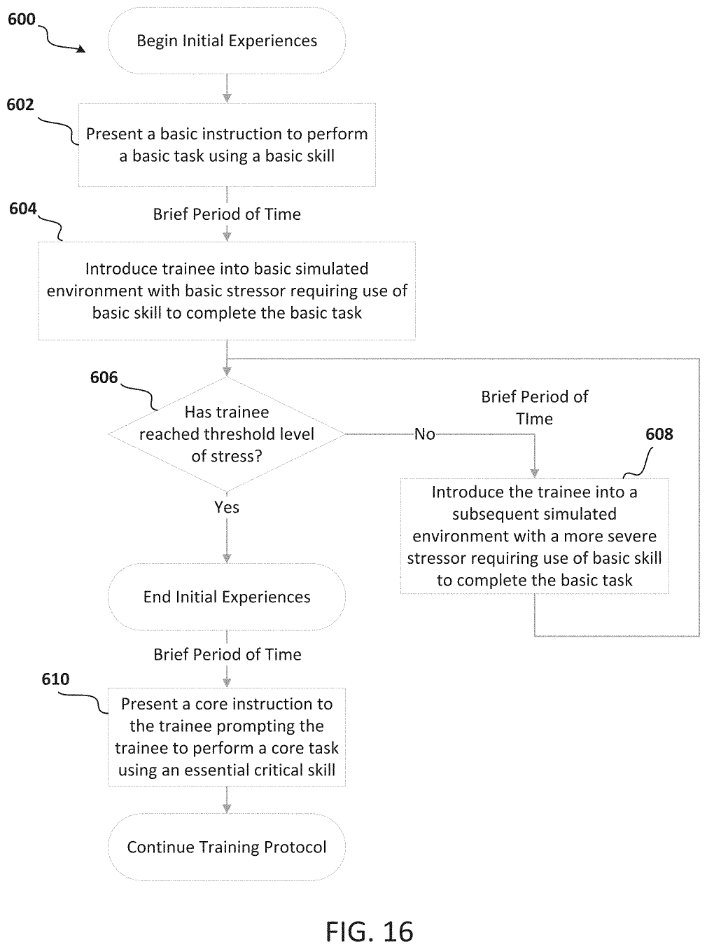

In at least some illustrative methods, the design of each experience may be established during the development process, before the installation process begins, based on known or estimated trainee characteristics and optional testing with individuals representing the trainee. Another illustrative method 600 for ensuring that a threshold level of stress is experienced by the trainee is shown in FIG. 16. A method similar to illustrative method 600 may be used in any one or more phases of the installation process to ensure a certain level of stress is experienced by the trainee during any one or more simulations.

In the illustrated embodiment, the method 600 is used to introduce initial experiences beginning with the presentation of a basic instruction to perform a basic task using a basic skill in step 602. In some embodiments, the basic skill is related to the critical skill path (e.g., an aspect of an essential critical skill).

Within a brief period of time, the trainee is introduced into a basic simulated environment exposing the trainee to a basic stressor and requiring the trainee to use the basic skill to successfully perform the basic task in step 604. The basic stressor is preferably designed to elicit a baseline stress response state in the trainee. Also, the basic simulated environment is preferably designed to favor successful performance of the basic task by the trainee in the basic stress response state.

The trainer is preferably trained to examine cues indicating the trainee's psychophysiological state (e.g., stress response state). In step 606, the trainer determines whether the trainee has reached at least a threshold level of stress. The trainer may make the determination after completing the basic task in the basic simulated environment. However, the trainer may additionally or alternatively make the determination before the trainee completes the basic task. For example, the trainee may have a slow respiration rate and relaxed muscles indicating to the trainer that the trainee is insufficiently stressed to facilitate subconscious installation of skills.

If the trainee has not reached the threshold level of stress, the trainer may introduce the trainee into a subsequent simulated environment in step 608 within another brief period of time after the trainee completed the previous task. In the subsequent simulated environment, the trainee is preferably exposed to a more severe stressor and requiring the trainee to use the basic skill to successfully perform the basic task. The subsequent stressor may be designed to elicit a more severe stress response state in the trainee relative to the stressor in the previous simulated environment (e.g., a basic stressor). Also, the subsequent simulated environment is preferably designed to favor successful performance of the basic task by the trainee in the baseline stress response state.

The method 600 may continue iteratively to repeat steps 606, 608 until the trainee has reached at least the threshold level of stress.

Once the trainer has determined that the trainee has reached at least the threshold level of stress in step 606, the initial experiences end (which may only be one experience in some cases). Then, within another brief period of time after completing the basic task, the trainer may present a core instruction to the trainee, prompting the trainee to perform a core task using an essential critical skill of the critical skill path in step 610 to continue the training protocol. However, another type of instruction may be presented if the method 600 is used in a different phase of the installation protocol (e.g., advanced comprehensive simulation phase or escalating comprehensive simulation phase). In this manner, at least a threshold level of stress is preferably ensured for the installation of skills.

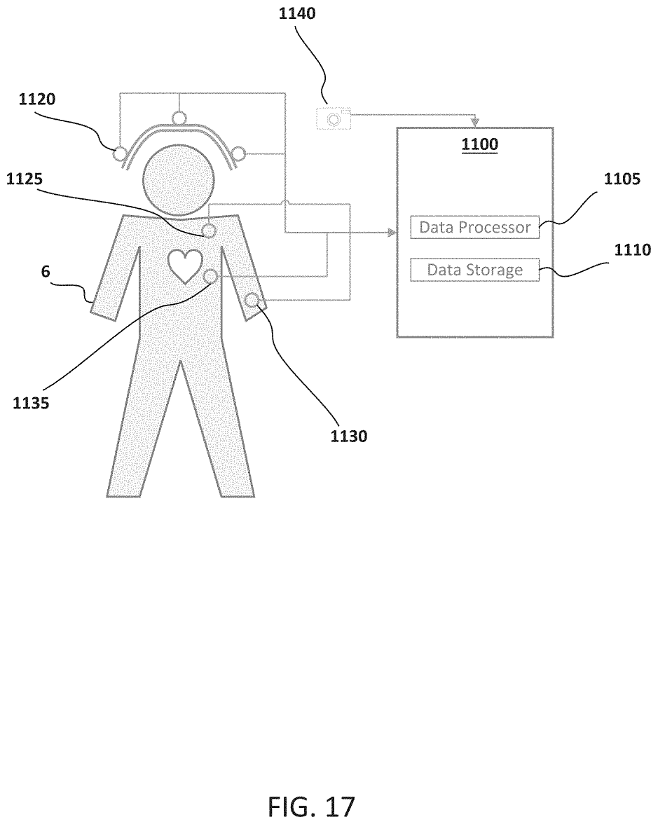

The observation of an internal marker may be facilitated by sensors, for example, as shown in FIG. 17. Non-limiting examples of internal markers relate to electrical signals, respiration, blood pressure, blood flow, galvanic skin reaction, and blood chemistry. In a schematic representation, a trainee 6 is outfitted with one or more sensors to facilitate the capture of markers. Although only a handful of sensors are shown, any suitable types and numbers of sensors for monitoring internal or external markers may be used. Some sensors may be disposed externally on or at a distance from the trainee 6, while others may be disposed internally or be implanted. Further, some sensors may be in contact with the trainee 6 on the head, torso, or limbs, for example, while other sensors may observe from a distance.

Various types of sensors may be used to monitor for potential markers. As illustrated, brain activity sensors 1120 are arranged around the head to monitor neurological activity in the trainee, such as event-related potentials (ERPs). Multiple sensors 1120 are preferably arranged on a cap donned by the trainee, for example. ERPs represent responses to thought or perception in the brain (e.g., internal or external stimulus). A galvanic skin response (GSR) sensor 1125 may be placed on the trainee to monitor perspiration. An accelerometer sensor 1125 may be placed on the trainee to monitor gross motor or fine motor movement of a limb, for example. An electrophysiological sensor 1135 may be placed on the trainee to monitor electrical signals to infer respiration, such as heart rate, for example. Non-limiting examples of electrophysiological sensors 135 include electroencephalogram (EEG), electrocardiogram (EKG), electrooculogram (EOG), and electromyogram (EMG) sensors. A camera 1140 is placed at some distance from the trainee to monitor external cues, such as motor movements or facial expressions. In other embodiments, a camera may be mounted to the trainee to monitor movements and positions, as well (e.g., mounted to the head or to a weapon to be held by trainee). Although only a certain number and type of sensors are shown, any suitable type and number may be used to monitor for internal or external markers. Further explanation and examples of sensors to collect markers of psychophysiological states may be found, for example, in U.S. Pat. No. 9,173,582, entitled “Adaptive Performance Trainer,” which is incorporated herein for all purposes.

In a marker monitoring apparatus, the sensors preferably are operatively coupled a computing device for storage and processing of sensor data. As shown, a training apparatus in the form of an illustrative automated adaptive tutor 1100 includes a data processor 1105 and a data storage 1110. The automated adaptive tutor 1100 may process and store data about observed markers from sensors or the trainer. The automated adaptive tutor 1100, as shown, may also be described as an automated marker monitoring apparatus, particularly when the tutor 1100 is configured to monitor for a marker in the trainee.

The techniques described in this disclosure, including those attributed to an automated adaptive tutor, marker monitoring apparatus, training apparatus, or the like, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in automated tutors, adaptive tutors, monitoring apparatus, training apparatus, interfaces, administrators, stimulators, sensors, monitors, data storage, data processors, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices, and techniques described in this disclosure may be embodied as instructions on a computer-readable medium such as RAM, ROM, NVRAM, EEPROM, FLASH memory, magnetic data storage media, optical data storage media, or the like. The instructions may be executed by one or more processors to support one or more aspects of the functionality described in this disclosure.

In various embodiments, the trainer may develop a “learning under stress” model that preferably associates one or more parameters with a critical skill. In many illustrative embodiments, such associations are preferably entered into, stored on, and retrieved by the automated adaptive tutor 1100. These associations may be stored in the data storage 1110, for example.

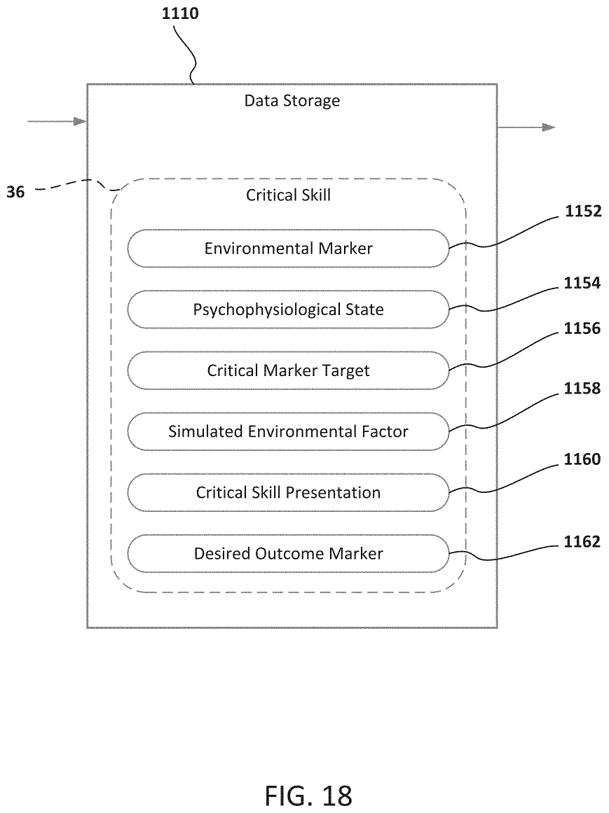

With reference to the illustrative embodiment shown in FIG. 18, the data storage 1110 is preferably configured to associate one or more parameters of the learning under stress model, such as associating a critical skill 36 with an environmental marker 1152, a psychophysiological state 1154, a critical marker target 1156, a simulated environmental factor 1158, a critical skill presentation 1160, and a desired outcome marker 1162. Parameters are may be determined based on experience or training of the trainer 4 and entered into data storage 1110, for example.

In various illustrative embodiments, the trainer 4 designs each simulation in an installation protocol is designed to install at least one critical skill 36 by requiring the trainee to use the critical skill to complete a task or to achieve a goal. In many cases, through experience or prior training, the trainer 4 develops these simulations prior to the installation process to each include at least a simulated environmental factor 1158 and a critical skill presentation 1160.

The simulated environmental factor 1158 is designed to simulate, in sufficient fidelity, real-world environmental factors that elicit a psychophysiological state 1154 that a trainee is likely to experience when using the critical skill 36, for example, along a critical skill path. In many illustrative embodiments, simulated environmental factor 1158 is preferably designed to elicit a psychophysiological state 1154 including a sufficient stress response to subconsciously install the critical skill 36, and thus, is likely to influence the presence of the critical marker in the trainee.

A simulated environmental factor 1158 may represent any aspects of the simulation. The simulated environmental factors 1158 may correspond to factors in real-world environments or may be artificial by design. Non-limiting examples of simulated environmental factors 1158 include: a task, an objective, a stressor, a threat, a protectee, a visual presentation, an aural presentation, an electrical stimulation, an administered psychophysiological agent, a required critical choice, a required critical skill, a required critical skill aspect, coaching, a type thereof, a level thereof, and a number thereof.

The critical skill presentation 1160 is preferably designed to experientially present the critical skill 36 to the trainee for installation and use. The critical skill presentation 1160 may be performed by the trainer 4 while the trainee is exposed to the simulated environmental factor 1158 or beforehand within a brief period of time, so that the critical skill is preferably embedded in the subconscious mind of the trainee.

After the trainee is introduced into a simulation, the trainer 4 may monitor for signs of a critical marker reaching a critical marker target 1156, which indicates that the trainee is in the proper psychophysiological state 1154 for installation of the critical skill 36 along the critical skill path. In many cases, the experience or training of the trainer 4 is used to determine the critical marker to monitor for the particular simulation (e.g., critical skill 36 and psychophysiological state 1154) and the associated critical marker target 1156. For example, the trainer 4 may observe the extent that the trainee's pupil dilates as a critical marker. Once the pupil dilation reaches or exceeds a certain level, as determined by the trainer 4, the critical marker may be considered as meeting the critical marker target 1156.

The trainer is preferably skilled in identifying whether the critical skill was used to successfully complete the simulation, as well, to determine a desired outcome marker 1162. The desired outcome marker 1162 preferably indicates that the simulation was completed successfully using the critical skill 36. For example, a desired outcome marker 1162 for a targeting simulation requiring the critical skill of aiming at mid-range could be a registered simunitions hit to a human target's torso or a hole left by a real ammunition hit in a cut-out target.

Many illustrative simulations are preferably designed to favor success. For example, the trainer 4 may design the simulation with simulated environmental factors 1158 within the capabilities of the trainee based on predetermined characteristics of the trainee. Trainee characteristics may be based on individual traits or collective traits of a trainee population. Non-limiting examples of trainee characteristics include: a prior experience, a psychophysiological state response to a specific stressor, a psychophysiological state response to a specific skill, a measured baseline marker, a cognitive trait, and a physiological trait. Furthermore, a critical marker target 1156 may be calibrated in response to the particular characteristics of a trainee to customize a training protocol that was designed for a trainee population to fit the individual trainee.

Trainee characteristics may be elicited through a variety of techniques. In one example, one or more interviews with a representative sample of a trainee population may inform of likely prior experiences, likely baselines and responses to stressors or skills, and other cognitive or physiological traits (e.g., risk-tolerance, decision-making tendencies, age, height, build, etc.). In another example, a preliminary installation protocol may be tested on a representative sample of trainees to determine or verify trainee characteristics and, also, preferably to prove the efficacy of the preliminary design.

In many illustrative embodiments, the trainer 4 may enter the one or more parameters of a learning under stress model based on experience or training into the automated adaptive tutor 100 for storage. In addition, parameters may be further determined or updated as more trainees undergo the training protocol. For example, the trainer 4 may monitor for and determine that a particular critical marker target 1156 for the critical skill 36 is a response to a particular simulated environmental factor 1158 and critical skill presentation 1160 of the introduced simulation, which may be stored as associated in the data storage 1110. A psychophysiological state 1154 based on trainer experience may then be inferred and associated with the critical skill 36 and critical marker target 1156 in the data storage 1110.

Similarly, based on the outcome of the simulation, such as trainee performance and level of stress response elicitation, the simulated environmental factor 1158, critical skill presentation 1160, and even the desired outcome marker 1162 may be updated or adjusted to further refine the learning under stress model. In one example of updating a desired outcome marker 1162, although the desired outcome of hitting a target accurately may be the same, the desired outcome marker 1162 may be adjusted from hitting the head of a target to hitting the torso, for example, when the simulation outcome indicates that the critical skill presentation 1160 is insufficient to facilitate the skill required for achieving such a high level of accuracy. The adjustment may be warranted when calibrating the simulation design for trainee characteristics, for example. Alternatively, with similar results, the critical skill presentation 1160 may be updated to install the skill required to achieve the existing desired outcome marker 1162, instead of revising the desired outcome marker 1162. The trainer 4 may have discretion based on experience to determine the appropriate updates to the learning under stress model.

In at least some illustrative embodiments, the sensors coupled to the automated adaptive tutor 100 are may be used to monitor some or all markers. The tutor 1100 may be capable of providing data to the trainer 4 for identifying one or more parameters. For example, a heartbeat or pulse rate may be detected by the electrophysiological sensor 1135 as the trainee successfully performs the critical skill 36 in a simulation. In response to the data, the trainer 4 may determine a threshold level for the heartbeat of a trainee associated with successful performance of the critical skill 36 for storage as critical marker target 1156. Other parameters may be determined or updated accordingly.

In this manner, the trainer 4 may develop simulations for an installation protocol and may continue to refine the installation protocol and underlying learning under stress model as the trainer gains more experience with deploying the installation protocol to trainees. The refinements are preferably stored in an automated adaptive tutor 1100 for later recall or use. One application of later use is the refinement of the present or subsequent simulation in the installation protocol by the automated adaptive tutor 1100.

In at least one illustrative embodiment, the trainer 4 includes the automated adaptive tutor 1100. In other words, some or all of the functions of the trainer 4 are carried out by an automated machine rather than a human trainer. The trainer 4 if present may have a reduced workload. For example, the automated adaptive tutor 1100 may monitor markers, identify and record critical marker targets, monitor critical markers and desired outcome markers, present critical skills, or introduce simulated environmental factors.

In some illustrative embodiments, the tutor 1100 may be configured with the experience of a skilled human trainer in order to carry out these functions. The associations between parameters, for example, may be stored and associated in a table, with some parameters corresponding to inputs into the model and others being outputs from the model. In various illustrative embodiments, the tutor 1100 may be configured with a learning model, which may utilize any suitable learning algorithm known to one having skill in the art and the benefit of this disclosure, calibrated by the trainer's identification of critical marker targets 1156 and designed to mimic or predict the trainer's choices. Such a tutor 1100 may even be capable of identifying critical marker targets 1156 for new critical skills, trainees, and environments.

In at least one illustrative embodiment, the tutor 1100 may identify a critical marker by monitoring markers while a trainee undergoes an illustrative installation protocol. A critical marker may be identified based on changes in the markers before, during, or after installation of the critical skill (e.g., successful performance of a task with a critical skill). For example, data from the brain activity sensor 1120 may represent a significant change in an ERP in a certain area of the brain after the simulation compared to before the simulation. The change in brain activity may be considered a critical marker associated with the critical skill.

In at least some embodiments, the before-simulation marker and after-simulation marker are associated with respective psychophysiological states. The psychophysiological states may be inferred and associates with various markers or marker values, for example. In at least one illustrative embodiment, a critical marker may be described as a change detected from one state to another.

Once a critical marker has been identified and measured, a critical marker target 1156 may be stored in the data storage 1110. A critical marker target 1156 may be stored in various manners to indicate the presence of a critical marker when compared to a measured marker value. Non-limiting examples of a critical marker target 1156 include a marker presence value (e.g., true or false), an absolute marker value (e.g., a level beyond which the marker is present), a marker change value (e.g., a difference between before and after installation), a marker duration value (e.g., the length of time a marker is present or exceeds a value), a relationship to another marker value (e.g., value compared to another measured marker), or combinations thereof.

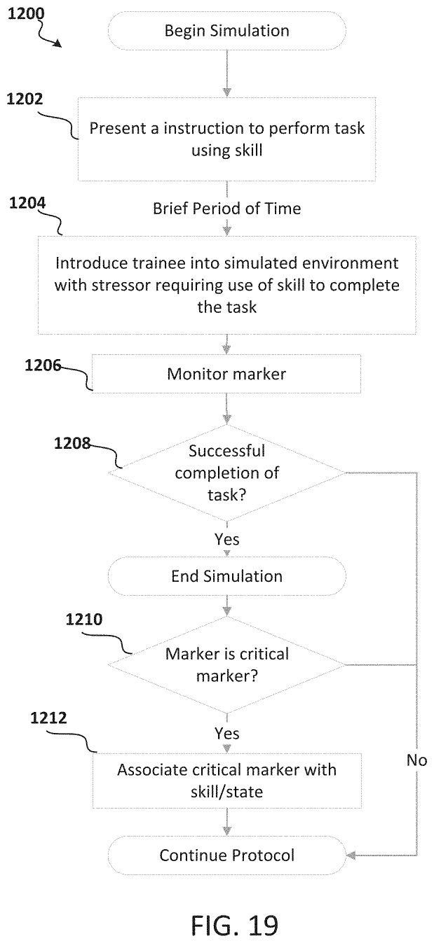

One example of developing a learning under stress model is process 1200 shown as a schematic representation in FIG. 19. For a simulation, the process 1200 begins with presenting a trainee with an instruction to perform a task using a skill or achieve a goal in step 1202. After a brief period of time, the trainee is introduced into a simulated environment with a stressor requiring use of the skill to complete the task or achieve the goal in step 1204.

Markers of the trainee are monitored in step 1206. In some embodiments, certain markers may be monitored in response to the particular critical skill being installed. Markers may be monitored before, during, and/or after the simulation. The successful completion of the task marks the end of the simulation in step 1208. If the task or goal is not successfully completed, then none of the markers may reliably be determined to be critical markers based on the outcome of that particular simulation. However, if the simulation is successfully completed, one or more markers may be considered to represent the learning or use of the skill or with an underlying psychophysiological state in step 1210. The marker is preferably determined to be a critical marker and associated with the corresponding skill or state in step 1212.

The installation protocol may continue after process 1200. For example, another pair of instructions and simulations may present a different task, skill, and stressor to the trainee. This process may continue until all critical skills are installed, for example.

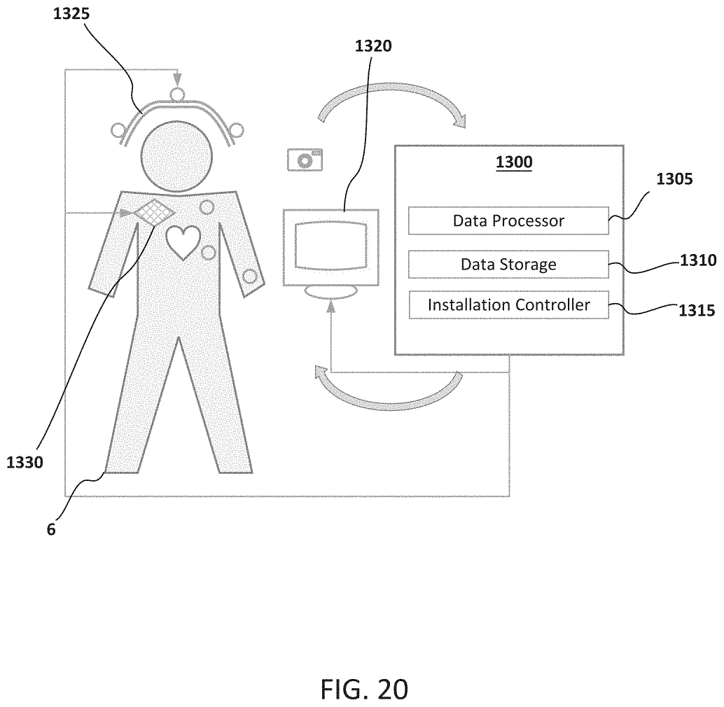

In some cases, the tutor 1100 may also be configured to present some or all critical skills and simulated environmental factors. As shown in FIG. 20, an illustrative automated adaptive tutor 1300 includes a data processor 1305, a data storage 1310, and an installation controller 1315. The data processor 1305 and data storage 1310 may be configured similar to data processor 1105 and data storage 1110. Further, the tutor 1300 may be operatively coupled to critical marker sensors as shown in FIG. 17 (not shown here) to receive sensor data. In many illustrative embodiments, however, various simulated environmental factors may not be provided by the tutor 1300, such as a live human target or protectee.

As shown, tutor 1300 may differ from tutor 1100 by the inclusion of the installation controller 1315 configured to provide one or more simulated environmental factors. In various illustrative embodiments, the controller 1315 is operatively coupled to the data processor 1305 to receive commands, such as a command to provide an environmental factor. An illustrative installation controller 1315 may be configured to provide simulated environmental factors through a virtual reality interface 1320, an electrical stimulator 1325, and/or an agent administrator 1330, as non-limiting examples. A virtual reality interface 1320 could include, for example, at least one of an image generator, a sound generator, a motion generator, and a haptic-sensation generator. An image generator could be a headset, monitor, screen, or interface with the visual cortex. A sound generator could be a headphone, speaker, Foley device, or interface with the auditory cortex. A motion generator could be a moving object at a distance, a simulated transport for the trainee, or interface with the vestibular system. A haptic-sensation generator could be a vibrator coupled to the body, a pressure cuff or suit, or an interface with the somatosensory system.

In some embodiments, the installation controller 1315 may be integrated, into a single neurological interface with the trainee that consciously or subconsciously presents the simulated environmental factors or mimics their effect on the neurological system (e.g., deep brain stimulation), for example, via electrical stimulator 1325. In other words, the installation controller 1315 may bypass the sensory system of the user and directly simulate the neurological effect in the brain. In this manner, the simulated environmental factor may be presented subconsciously or consciously to the trainee. An illustrative electrical stimulator 1325 may include an electrode electrically coupled to the trainee to deliver electric signals to the neurological system of the trainee and may include an electrical signal generator to deliver electrical signals to the electrode.

In various embodiments, a psychophysiological agent may be administered through some administering connection (e.g., intravenous delivery system) by the agent administrator 1330. The psychophysiological agent may be a chemical or biological agent capable of temporarily altering the stress response state of the trainee, for example, by raising perspiration and heart rate. In this manner, the psychophysiological state of the trainee may also be altered consciously or subconsciously.

The learning under stress model may be used to improve training. For example, the one or more stored parameters in the data storage 1110 may be used to verify the psychophysiological state during the simulation by comparing the critical marker target 1156 to a measured critical marker to determine the presence of the critical marker. If the critical marker is present, the critical skill may be optimally via the simulation. However, if the critical marker is not present, the training protocol can adapt to further facilitate the presence of the critical marker and, accordingly, to further facilitate the subconscious installation of the associated critical skill.

In at least some illustrative embodiments, the training protocol may adapt by modifying a simulated environmental factor in the current simulation. In one embodiment, before a task is completed or a goal achieved, a simulation factor may be adjusted from a level, type, or number that did not facilitate the presence of the critical marker to a different level, type, or number to facilitate the presence of the critical marker. For example, if one potential target is insufficient to elicit a psychophysiological state and the presence of a critical marker, two or three potential targets may be introduced as factors in a simulation to increase a stress response state. In other words, a subsequent simulated environmental factor may be determined that is different than the initial environmental factor.

In various illustrative embodiments, the training protocol may adapt by modifying a simulated environmental factor for a subsequent simulation. In one embodiment, if a task has been completed or goal achieved, thereby ending the current simulation, a subsequent simulation may be introduced having a subsequent environmental factor (e.g., modified from the environmental factor previously designed for the subsequent simulation) to further facilitate the presence of the critical marker. The subsequent simulation may also require use of the same critical skill to ensure proper installation. In some cases, additional simulations may be added with the subsequent environmental factor, for example, to ensure that the critical marker is present before installing additional critical skills. In at least some embodiments, at least three skills are required to be used in each simulation, even when a critical skill installation is repeated.

In further illustrative embodiments, the training protocol design may be adapted for subsequent trainees. Other training protocols may also be adapted, as well. For example, another training protocol for a different critical skill set may be modified where the same psychophysiological state is desired from a similar trainee population.

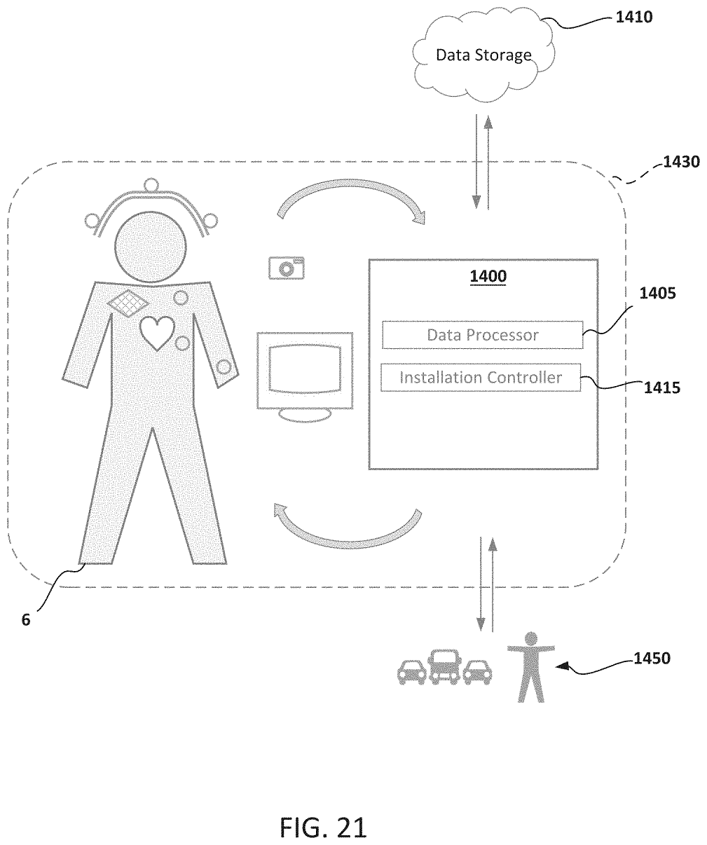

The learning under stress model may also be leveraged in real-world environments to facilitate on-demand installation and performance. As shown in FIG. 21, a training apparatus is shown in the form of an illustrative automated adaptive tutor 1400 including data processor 1405, data storage 1410, and installation controller 1415, which are similar to the similarly-named elements shown in FIG. 20. The tutor 1400 is configured to provide simulated environmental factors to the trainee 6 and to receive sensor data about the trainee 6.

However, the data storage 1410 may be located remotely from the trainee 6. As shown, the data storage 1410 is located outside of environment 1430. This may be useful when the data storage 1410 cannot physically be present in environment 1430 or when the data storage 1410 is receiving information from other environments in distributed geographic locations, for example. The data storage 1410 may be coupled to the other components of the tutor 1400 via techniques known to those having skill in the art, such as via a wired connection, wireless connection, or a combination thereof, including direct and indirect connections (e.g., network). The data storage 1410 is in operative communication with the data processor 1410 to receive requests for information and to provide requested information thereto.

In the environment 1430, the trainee 6 may be required to perform a task or to achieve a goal with real-world consequences. In other words, the trainee 6 may be asked to perform critical skills in response to real-world environmental factors. In some cases, the trainee 6 may not possess all of the critical skills necessary to respond to the real-world environmental factors to achieve success. The learning under stress model and tutor 1400 may be configured to install critical skills as needed.

In some illustrative embodiments, the trainee 6 may be able to consciously identify the task, goal, or even critical skills required to achieve success. In such cases, the tutor 1400 may be able to install the critical skill on-demand and in real-time (e.g., in time for useful application of the critical skill in response to the real-world environment). For example, the tutor 1400 may be configured to receive a conscious request from the trainee 6 and to present the appropriate critical skills. In addition, the tutor 1400 may have simulated environmental factors stored and associated with those critical skills and may be further configured to expose the trainee to those factors to facilitate the installation of the critical skills presented.

In some cases, the real-world environmental factors are sufficient to elicit the appropriate psychophysiological state in the trainee to meet the critical marker threshold. The tutor 1400 may not expose the trainee to simulated environmental factors in such cases, and may only present the critical skill required.

In various illustrative embodiments, the trainee 6 may be consciously unaware of the critical skills needed to complete the task or to achieve success. In such cases, the tutor 1400 may be able to discern the environmental factors and select the critical skills that will facilitate success. For example, environmental factors may be determined in response to environmental sensor data or in response to markers in the trainee corresponding to certain environmental factors. In at least one embodiment, the real-world environmental factors may be discerned from an interface with the neurological or sensory system of the trainee who experiences the environmental factors and generates signals in response thereto (e.g., ERPs). For example, a simulated environmental factor may be generate a certain ERP, which may also be generated by the real-world environmental factor that the simulated factor was designed to represent.

The tutor 1400 may store environmental markers in the data storage 1410, for example. In many embodiments, the environmental markers are compared to the real-world environmental factors discerned (e.g., from sensor data or the trainee interface). For example, an environmental marker may be the sound of a gunshot. An environmental sensor that detects a sound may generate data that is compared to the potential environmental markers stored in the tutor 1400. In response to the comparison with the environmental marker, an associated environmental factor may be determined to be present. The environmental marker is preferably associated with a critical skill in the data storage 1410 or determined to be associated by the data processor 1405. In response, the associated critical skill is preferably presented to the trainee 6 for real-time installation.

In various embodiments, the critical skill may further be determined in response to a critical marker being present. For example, the sound of a gunshot as the detected environmental marker in addition to a pupil dilation critical marker may indicate that the trainee 6 has visually identified a potential threat. The cognitive strategy skill of determining whether a potential threat is a bonafide threat may then presented for installation and use.

In some cases, the trainee 6 may not be in the real-world environment. In other words, the environment 1430 may not be the physical location of the mission. In such cases, the tutor 1400 may be configured to receive environmental information from remote sensors, which may be in operative communication in any suitable manner known to one having skill in the art. For example, a live video and audio stream may be presented to the trainee 6 via a satellite feed that represents a real-world environment with remote forces 1450, such as soldiers or drones, to which the trainee is tasked with issuing orders to complete the mission. In many illustrative embodiments, the tutor 1400 is preferably configured to present the real-world environmental factors as simulated factors to the trainee 6. The tutor 1400 may also be able to receive the trainee's orders via the interface with the trainee 6 (e.g., neurological interface or movement sensors) and relay the orders to the remote forces 1450.

In this manner, the critical skills are capable of being subconsciously installed by the tutor 1400 on-demand and in real-time for the trainee to use. This arrangement allows the trainee 36 (e.g., a commander) to have access to a vast set of critical skills that may be stored on a remote supercomputer that can also analyze and identify critical skills for installation in the trainee 36. With the critical skills installed and being in a psychophysiological state facilitated by the tutor 1400, the trainee 36 may incorporate human decision-making processes while utilizing the critical skills. For example, the trainee 36 may exercise judgment in response to human factors, such as public relations or inter-governmental responses to certain actions. In other words, the tutor 1400 may be limited to installation of skill for saving lives, but may be limited in emotional intelligence or more complex human interactions that have yet to be modeled.

The learning under stress model may have further implications and applications. For example, although in some embodiments the learning under stress model is designed to be specific to a trainee or trainee population, the collection and analysis of a sufficient number and diversity of trainees may facilitate the generalization of a learning under stress model that can then be applied to all individuals. Such a model may have many future implications in enhanced cognition and enhanced man-machine interfaces. Furthermore, with the development of artificial intelligence that attempts to model human cognition, having such a robust learning under stress model may provide an important piece to developing a complete artificial intelligence. Even further, the performance of an artificial intelligence may be enhanced by applying the learning under stress model to identify the most effective manner of installing new skills.

While the present disclosure is not so limited, an appreciation of various aspects of the disclosure will be gained through a discussion of a specific example and various illustrative embodiments provided below.

### Example

The following example is provided to show that illustrative training methods of the present disclosure have been used to install various sets of skills into multiple trainees. Various illustrative installation method were developed for training military, law enforcement, and private security service critical skills. The illustrative installation methods incorporated models of learning, perception, and performance according to psychological and cognitive neuroscience principles. Although other methods of training in the arena of teaching skills for use under stress, which are widely known and used, the following example shows that the illustrative installation methods may have facilitated superior accelerated learning and lasting retention of skills for performance under stress.

In this example, a study incorporating mixed-methods examined illustrative installation protocols to gain understanding regarding the perception of past trainees on the impact of the illustrative training method on their personal and professional lives and to identify variables that differentiate the illustrative training method from similar trainings.

Method.

The mixed-methods design included qualitative and quantitative data collection and analysis.

First Part: Semi-Structured Interviews.

The first part of the study included semi-structured interviews. Qualitative data was collected from a sample of four participants who were past trainees who completed in-depth, semi-structured interviews about their experience with at least one illustrative installation protocol. The interviews were designed to facilitate understanding of the installation protocol and to facilitate the design of a training-specific survey to more thoroughly investigate trainee experiences.

The participants were selected via a convenience sample and completed interviews with the investigators. The number of installation protocols completed by each trainee ranged from 1 to 7, and the length of time since completing the most recent installation protocol ranged from 2 weeks to over 10 years. All installation protocols involved the same trainer, Marcus Wynne).

Interview responses were transcribed and analyzed using a 7-step phenomenological approach. In a first step, the two investigators read and reread the transcripts to independently identify key ideas generated by the trainees. The identified key ideas were coded into tentative labels in an attempt to capture the essence of the ideas of each transcript. Then, in a second step, the tentative labels of the investigators were compared and common labels were identified for further analysis.

In a third step, one investigator coded the transcripts using the identified common labels. Next, in a fourth step, the other investigator checked the coding of the transcripts to remove areas with differences in coding from the analysis and generated a set of remaining labels.

For step five, the remaining labels were grouped into broad categories with themes by one investigator. For step six, the other investigator checked the categories and themes to remove ideas or statements that were deemed ambiguous.

Finally, in step seven, written descriptions of all categories and themes were created and revised by the investigators. Throughout the qualitative analysis, the investigators took steps to promote trustworthy collection, evaluation, and presentation of interview data. Assumptions and predispositions about the research and data collected were identified and acknowledged to facilitate setting aside individual assumptions and biases. Further, credibility and confirmability were encouraged through consensus building and debriefing processes between the investigators, as well as multiple trainee checks throughout the interview procedure.

Second Part: Training-Specific Surveys.

The second part of the study included training-specific surveys. Quantitative data was collected from the survey responses of 67 participants, including 32 trainees and 35 non-trainees. Non-trainees were defined as individuals with similar backgrounds who had not participated in an illustrative training method. The survey was designed to facilitate comparison of trainees with non-trainees on measures of situational awareness, stress inoculation, state management (e.g., stress management), and cognitive processes (e.g., cognitive strategy). Measures and items created for the survey were informed by relevant literature and qualitative findings from the qualitative analysis.

The participants included those who were sent emails including a link to an on-line survey requesting participation and completed the on-line survey. The investigators sent emails to known trainees to create the trainee group. The non-trainee group was created through multiple efforts: emails to trainees also asked the trainees to share the link with a colleague who was a non-trainee and emails were sent to contacts in police and military organizations.

Demographic information for participants can be found in Table 1. Trainees had completed an average of 3.5 trainings (Mhours=34.77, SD=35.30) approximately 5.38 years ago (SD=5.92), had an average of 19.72 years on the job (SD=8.37), and were US (56.25%), European (37.50%), or other (6.25%) nationals. Non-trainees had completed an average of 18.5 hours (SD=31.10) of other trainings perceived to be “brain-based”, had an average of 14.71 years on the job (SD=11.58), and were US (85.71%), European (5.71%), or other (8.57%) nationals. All participants provided on-line, informed consent.

TABLE 1
Demographic Information for each participant
group. “Other” occupations included
martial arts, technology, construction, and law.

| | | | Occupation | | | |
|---|---|---|---|---|---|---|
| | Age | Gender | | Law | | |
| | M(SD) | M/F | Military | Enforcement | Security | Other |
| Trainees | 46.84 | 29/3 | 12.5% | 25% | 25% | 37.5% |
| | (9.10) | | | | | |
| Non- | 43.26 | 35/0 | 34.29% | 37.14% | 11.43% | 17.14% |
| Trainees | (10.97) | | | | | |

The training-specific survey was developed based on the illustrative training method as well as results of a phenomenological qualitative analysis conducted on the semi-structured interviews (see Appendix B). The survey consisted of 34, 5-point Likert scale items, as well as 5 open-response items. The first 17 items asked participants to rate their perceived abilities in situational awareness, state management, cognitive ability, and coping in times of stress/threat vs. non-stressful times (1=poor, 5=excellent). The second 17 items asked the participants the same questions, but this time they were to rate their perceived impact of their different training experiences on these abilities (1=not at all influential, 5=extremely influential).

Participants also completed two standardized measures of stress and anxiety. The Perceived Stress Survey (PSS) assesses the degree to which one perceives life events as stressful. It consists of 14 items rated on a 5-point Likert scale from 0 (never) to 4 (very often). The trait subscale of the State-Trait Anxiety Inventory (STAI) assesses general and long-standing feelings of anxiety, most characterized by worry. It consists of 20 items rated on a 4-point Likert scale from 1 (Not at All) to 4 (Very Much So).

Qualitative analysis of open-response items and quantitative analysis of Likert-type measures were performed. Qualitative analysis was performed on five open-ended questions was included in the survey (Appendix B). Given the interactive nature of the study, qualitative findings from the interview portion of the study provided a foundation for and informed analysis of survey responses. Namely the categories and themes that emerged from the in-depth interviews were used as a framework for understanding and analyzing the brief open-ended survey responses. The goal of qualitative analysis remains exploratory in the second part of the study with the intention of informing future research rather than confirming a coding system or explaining participants' experiences. Thus, similar to the first part of the study, standards for trustworthiness of qualitative research are followed and consensus building and debriefing procedures were used between two investigators throughout the analysis process.

In step 1, each investigator read the open-ended responses and coded the responses with initial labels derived from qualitative interviews. They each created new labels to capture data that did not fit with pre-determined labels. In step 2, investigators compared new labels as well as labels assigned to raw data until consensus was reached. In step 3, one investigator evaluated the new labels that emerged from the survey responses to see how they applied and/or fit into the categories and themes that emerged from the interview data. During this step, this investigator also evaluated the categories and themes from the interview data to see if they were consistent with and captured the raw data presented amongst survey respondents. This step resulted in addition and deletion of themes as appropriate. In step 4, the other investigator took the new themes back to the data to review applicability and consistency of the themes to the open-ended data. In step 5, investigators discussed the set of categories and themes and revised until agreement was reached regarding final version. In step 6, each investigator independently went back to raw open-ended item data and utilized final themes to code the data, allowing for all applicable themes to be applied to any given response. In step 7, they compared coding and made revisions until consensus was reached. In step 8, written descriptions of all new themes and nuances to pre-determined themes were created and revised by investigators and discussed herein. All illustrative training methods described by participants were taught by the same trainer.

In the quantitative analysis of Likert-scale items, summed scores for all stress/threat (S) and non-stressful (NS) items for both the perceived ability (PA) and perceived training impact (PTI) subscales were calculated, creating four dependent variables (PA-S, PA-NS, PTI-S and PTI-NS). For all subscales, one direct trainee provided incomplete information and was excluded from the analyses. For the PTI subscales, three additional direct trainees and 18 non-trainees provided incomplete information and were excluded from the analyses. For the PSS, two direct trainees and one non-trainee provided incomplete information, and for the STAI four direct trainees and four non-trainees provided incomplete information. Independent samples t-tests were conducted for each dependent variable. Due to the low completion rate for non-trainees, for the PTI subscales analyses the SPSS function “Select Cases” was used to randomly select 53% of the trainees to create equal samples sizes (17 participants in each group). All comparisons met Levene's test for equality of variances.

Results: Part 1, Semi-Structured Interview.

Analysis of the semi-structured interview data produced three broad categories: training experience, lasting state management, and pervasive impacts were distilled from the data. These categories and all themes in each are described below and accompanied by exemplary quotes. Quotes from participants were not edited for grammar. The letter P denotes participant and is followed by the respondent's research identification number. Frequency of occurrence for each category and theme are provided in Table 2.

TABLE 2
Frequencies for Each Theme and Each
Category by Participant and Overall.

| | P1 | P2 | P3 | P4 | Total |
|---|---|---|---|---|---|
| Training Experience | | | | | |
| Immersive | 3 | 3 | 4 | 1 | 11 |
| Dynamic | 0 | 4 | 1 | 0 | 5 |
| Unique | 2 | 2 | 3 | 1 | 8 |
| Total | 5 | 9 | 8 | 2 | 24 |
| Lasting State Management | | | | | |
| Physiological | 1 | 0 | 1 | 2 | 4 |
| Emotional | 7 | 1 | 6 | 5 | 19 |
| Cognitive Skills | 1 | 1 | 0 | 2 | 4 |
| Awareness | 5 | 2 | 6 | 3 | 16 |
| Total | 14 | 4 | 13 | 12 | 43 |
| Pervasive Impacts | | | | | |
| Ingrained Automaticity | 4 | 0 | 1 | 3 | 8 |
| Emotional Release and | 0 | 2 | 0 | 3 | 5 |
| Healing | | | | | |
| Teaching/Training | 1 | 0 | 3 | 1 | 5 |
| Others | | | | | |
| Total | 5 | 2 | 4 | 7 | 18 |
| | | | | Grand Total: | 85 |

Category 1: Training Experience.

The training experience category captures the collective ways in which interviewees experienced the neural-based training. This category is represented by the following themes: immersive, dynamic, and unique.

The immersive theme reflects participants' descriptions of the training as simulating “real life” situations and focused on learning by doing. All interviews suggested this theme. Participants noted the learning by doing aspect of their training, as expressed by one participant as she recalled the memorable aspects of her training experience, “he's [the trainer] more of a drop you in the pool so you can swim . . . he'll drop you in there and be like oh good you can swim, that's great . . . but then you know when you're out in the water you just swim, so you don't have to go well am I swimming correctly, how does everyone else think I'm swimming” (P3). Participants also spoke about the realistic and/or reality-based nature of the training. For example, while commenting on a recent training experience, one participant described her understanding of the reality of the scenario, “In this particular scenario it just feels a little bit more like we're working on this for a specific reason and that reason is to protect yourself and to do something safely and to really understand the reality of what these types of physical conflicts are like” (P2). Participants remarked specifically on how their training with the trainer simulated real life threat and/or stress compared to other trainings. In speaking to this comparison, one participant noted “he's [the trainer] like ‘okay do the skill under stress now’ and then he sort of adjusts from there.” (P3).

The dynamic theme highlights the fluid and dynamic nature of the training. While all participants alluded to the dynamic nature of the training, two of the four participants spoke directly to this theme. Participants identified the training as dynamic in regards to the movement (their own and others), skills addressed, type of scenarios, and types of roles being played. One participant described the variety of ways that the training was dynamic:

“We did a bunch of pistol training, I guess just a lot of movement and trying to be on target while there's movement, keeping a range between you and the aggressor, some instances of multiple opponents and trying to identify who would be a threat and who would be a victim of something, kind of trying to walk into a situation and make quick decisions, while effectively using a firearm basically. And also keeping mobile yourself, so we have this aggressor moving around and moving towards you or keeping its distance from you and just trying to close the distance to where you can properly use the weapon” (P2).

Another participant highlighted the dynamic and fluid nature of the training compared with other trainings, “He'll be clear but much more concise, and a little less boring, so it's a much more fluid environment I would say and it's much more dynamic and fluid I think” (P3).

With respect to the unique theme, interviewees noted that their training with the trainer was unique in comparison to other trainings in which they have participated. They referred specifically to the “brain training” or mental nature of the training as unique. In comparing the training with the trainer to other trainings, one participant spoke about the focus on mental state during, “I think [the trainer] is more about installing function within a specific state of mind, and the other trainings are designed to have you perform a specific skill, not necessarily with the state of mind, but they want you to have the skill, and he wants you to basically function effectively” (P3). Another participant highlighted the innovative nature of the state management aspect of the trainer's training compared to other trainers, “[The trainer] has been way ahead of these people [other trainers]; they'll always kind of talk the same talk, but it comes out in the actions of how we train and how we do our stuff . . . most tactical instructors don't focus on state management [and] go right into defensive tactics or weapons handling . . . Pretty hard to teach somebody how to manage their own emotional state, especially under immediate threat to life situations. So no I really haven't trained with anybody like [the trainer] I wouldn't say, I would say some of the tactical concepts are the same and some of the shooting stuff or whatever is the same, but as far as the mechanism we're using I haven't encountered anything like that before” (P4).

Although participants unanimously highlighted the unique nature of the training, two of the participants indicated that they were not able to accurately differentiate between the influence of the training with the trainer and previous experiential learning, specifically their martial arts training. For example, one participant stated, “I personally feel like all the training I do at a martial arts gym is applicable. I think all those skill sets sort of came out in the training” (P2).

Category 2: Lasting State Management.

The lasting state management category encompasses the variety of ways that interviewees spoke about their enhanced ability to manage internal functioning while facing stress or threat. Not only did participants speak to diverse aspects of functioning (e.g. physiological, emotional, mental), they also spoke to both state (during the training and during specific moments of high stress in their personal and professional lives) and trait (long term change in their ability to function on a day-to-day basis) shifts in their functioning. The lasting state management category is comprised of the following themes: physiological state management, emotional state management, cognitive skills, and awareness

The physiological state management theme portrays participants' references to managing physiological aspects of their functioning. Participants spoke specifically to managing adrenal response and sensory functions as illustrated by the following quote, “tweaking the adrenal response, I think that's one thing I got from training with [the trainer] early was that I had a better understanding of adrenal response during threat-to-life things . . . in terms of like what's happening at different heart rates, auditory exclusion, tunnel vision, all that stuff” (P3). One participant described how physiological state management training was implemented, “he [the trainer] talked about controlling the tunnel vision under stress and so he introduced a stress again by the discharge of the weapon and the impact so you have the pain response, you have the auditory response, which normally you get the adrenaline dump and you instantly get tunnel vision if you're not prepared for it” (P1). Interviewees also spoke about their enhanced abilities in regards to physiological responses as a result of the training. For example, one participant noted, “in a steaming hot room I can engage in a fight or a gun fight if need be for hours on end; that's the physical portion of it” (P4).

Regarding the emotional state management theme, all four interviewees spoke of their improved ability to manage their emotional functioning during stress and/or threat to life as a result of participating in the trainer's training. Specifically they spoke about their abilities to control anger and aggression, manage fear and stay calm under threat, and experience long-term inoculation to stress. This theme incorporates interviewees' perceptions of training techniques as well as their applied use of emotional state management techniques. One participant spoke about the way that the training addressed fear and aggression simultaneously, “so when we talk about the fight and flight, instead of a lot of people would react to flee, he wanted you to react that fight response and bring your aggression level to be where you would be angry as you walked towards that person” (P2). Another interviewee spoke about a technique that he learned from the trainer to manage “bad” feelings:

“One of the techniques that he taught me was with the bad things, and it can be something as simple as somebody cutting you off or something that really gets under your skin, and you can visualize that but instead of having it be this huge thing in color that consumes your whole part of your brain, what you do is you take the color out of it, you turn it black and white, as we talk about it I can almost feel that feeling in my stomach of the emotional change” (P3).

In addition to talking about training techniques, interviewees spoke about examples of using the technique outside of the training. One participant spoke about eliciting aggression generally, “I mean it's great and I can kind of switch on and be scary if I need to” (P3). Another participant spoke about how the training influenced his response during a recent event, “I was able to take him down and stop the attack, and I was also able to stop myself from inflicting any further damage on the assailant even though at the time I was extremely angry” (P4). Finally, one participants spoke about the impact of the training on stress inoculation specifically, stating “training with [the trainer], there's always stress as well, but I think it's inoculated me to do these other training's especially over a long period of time and just sort of switch off my brain and just do the drills” (P3).

For the cognitive skills theme, participants collectively spoke of cognitive skills that were specifically addressed in their training, and described how these skills have impacted their functioning outside of the training. The cognitive skills theme includes visualization, memory, and decision-making skills. One participant described the process he has learned from the trainer to enhance memory, “play that training in your brain and he'll have you play it forward, and then he'll have you play it backwards, and then he'll have you play it fast-forward. So every little thing, every person you talked to, everything . . . ” (P4). Participants also spoke about specific examples of using cognitive skills in their work. One participant spoke about how the training applies in a real-life shooting scenario, “run through that event in your brain and you complete it in your brain before you've actually done it so you've already had a successful outcome before you've even pulled the trigger once . . . and then you actually go through the act itself so you just repeat what you just ran through exercise in your brain (P3).

As to the awareness theme, all interviews spoke about enhanced awareness of self and environment under stress and/or threat. The theme reflects a variety of awareness forms, including peripheral vision, situational awareness, general awareness of their environment, and awareness and management of internal responses. Throughout the lasting state management category, this theme represents participants' experiences both during the training and as a lasting result of the training. As one participant spoke about her experience of awareness during the training she noted, “you're very aware of yourself and your own personal safety and what the urgency of that really feels like” (P2). Participants spoke about the impact of the training on their lasting awareness both generally, and in regards to specific examples of their professional work. One participant spoke directly about the impact of the training on his environmental awareness, “it's [the training] increased my peripheral vision, so that's a huge part of awareness . . . learning how to read people, sensing tension, and the peripheral vision” (P4). Another participant provided specific examples regarding the impact of his training with the trainer on his success in the field. He stated, “I can watch you and watch your body mechanics and I can look at you and tell if you're carrying a gun and if a lot of things are bad or if you're setting out to do something . . . that's a lot I did learn through him [the trainer] . . . applying it to the shooting spectrum as far as watching people and just being aware of your surroundings” (P1). This participant went on to report, “Well I've been in a few gunfights, and one particular one where we were actually trapped, where the only way we were going to get out . . . we would have to kill a guy. But what I found is that I never lost my peripheral vision” (P1). Finally, participants spoke about enhanced awareness of their personal abilities and skills as a result of the training. One participant described this personal awareness and noted the relationship between awareness and confidence, “I am definitely more . . . aware of my reaction to things . . . I was more on target than I expected. Being aware of that kind of potential skill set is kind of cool in myself, it kind of affects your confidence a little bit” (P2).

Category 3: Pervasive Impacts.

The pervasive impacts category captures the ways that the training pervades and extends beyond the type of scenarios trained for, as described by trainees. This category reflects participants' expressions of the ways that their training with the trainer impacted their lives at a deep level and how it has impacted their work with others. The pervasive impacts category includes the following themes: ingrained automaticity, emotional release and healing, and teaching/training others.

The ingrained automaticity theme portrays the automatic nature of the skills gained in the training with the trainer, as described by participants. One participant talks about recognizing the ingrained nature of the training as he reflects back on the training and how he has used what he learned, “I reflect back, and back then I just took a training and it was ingrained in me and I don't think about it . . . I teach to tell people you've got to learn with your brain . . . it's not the physical aspect but you need to ingrain this stuff into your brain so your body would just subconsciously do it, where it's not a conscious effort anymore” (P1). Another participant spoke about his perception of the process of ingraining the information, “most of the time . . . I don't even know I'm doing it [skills from training]. He [the trainer] actually told me that would happen. He moved through the part where you're practicing the skill and you're learning the skill, and you move on to where you're consciously doing the skill, and then it evolves to subconsciously doing it, which like I said sometimes I don't even know I'm doing it until I look back” (P4). One interviewee described an example of automatically functioning and reacting when a stressful event occurred while she was leading a training, “but it was just kind of like okay here's the deal I've got 30 people under my care, this could go sideways, and I just did it; but that's from that training” (P3).

For the emotional release and healing theme, participants spoke about the impact of the training on their personal emotional well-being as well as healing from difficult events in the past. Three of the four participants contributed to this theme. Two participants spoke extensively of the emotional release and healing that resulted from their trainings with the trainer. One participant stated “there were still those skeletons in my closet so I had to be aware that they were there and I had to be aware of the triggers. So it [the training] made me aware of a ton of stuff personally, some pretty deep stuff” (P4). He elaborated on what he learned about himself and his past, “I learned that I don't need to be bound by anything in my past and the way that I was raised or the things that I was connected to doesn't make me, me ultimately, I learned that I can let go of all that garbage and move on” (P4). Another participant spoke about her response to a recent training with the trainer. She indicated, “some stuff that I've kept to myself just kind of popped out of my mouth . . . that also led to a whole lot of my personal walls crumbing, which needed to happen and has probably needed to happen for 30 years but it didn't” (P3). She talks about her perception of what contributed to her emotional release, and clarifies that it was a positive event, “so I think it was the additional stress or whatever it just put me in a world where I was kind of protecting some people and doing a bunch of things all at once and I kind of overloaded. Which was great, it was a blessing” (P3).

As to the teaching/training others theme, According to three of the participants, their training with the trainer impacted their training and teaching of others. One participant spoke of the utility of emotional state management training in her work with a women's self-defense program, “the emotional piece, I haven't heard a whole lot of people talk about it. I do put it in the women's self-defense program just because if anyone is going to be attacked they have to be able to function with that stress is in there” (P3). The same participant revealed that her training with the trainer is central to her own training mission, “I mean one of the reasons I keep teaching is that I have this training, and need it and they don't have it” (P3). Another participant provides two explicit examples of how he has used his training experience to help others, “I was able to do that [referring to teaching/training others] with some people that I worked out with, and not only that but I was able to observe some of the physical manifestations of them learning, so they would sit there and their head would go to the side and that's their brain in action, and I didn't know any of that before. I also taught a friend of mine that was going through some real hard times how to visualize and how to replace that stuff, it was pretty neat” (P4).

Results: Part 2, Training-Specific Survey, Qualitative Analysis.

The investigators initial qualitative coding and analysis of open-ended survey data resulted in modification of the pre-determined themes derived from the interview portion of the study (Table 3).

Within the training experience category, two new independent themes were identified:

accelerated and exceptional. within the lasting state management category, themes were primarily maintained given their consistency with raw open-ended item data. Only one change was made in this category, namely the combining of the physiological state management theme with the emotional state management theme, resulting in an integrated theme named psychophysiological state management. The investigators implemented this change as survey participants often spoke of such state management generally and it was unclear if they were referring to emotional/mental state or physiological state. Within the pervasive impacts category, one new independent theme was added, broad applicability. In addition the theme of emotional release and healing theme that had emerged in the interview data was not indicated within the survey results and was thus removed. Descriptions of added themes as well as nuanced survey findings amongst pre-determined themes are presented below.

TABLE 3
Frequency counts of different categories and themes
from the open-response items in part 2 of the study.

| | Count |
|---|---|
| Training Experience | |
| Immersive | 12 |
| Dynamic | 8 |
| Unique | 14 |
| Accelerated | 14 |
| Exceptional | 30 |
| Total | 78 |
| Lasting State Management | |
| Psychophysiological | 26 |
| Cognitive Skills | 16 |
| Awareness | 35 |
| Total | 77 |
| Pervasive Impacts | |
| Ingrained Automaticity | 7 |
| Teaching/Training Others | 5 |
| Broad Applicability | 11 |
| Total | 23 |

Category 1: Training Experience.

Within the training experience category, survey participants noted the immersive, dynamic, and unique aspects of their experience training with the trainer, consistent with the themes that emerged from interview data.

As to the accelerated learning theme, in addition to providing information about the immersive, dynamic, and unique aspects of their training, survey respondents noted that the nature of the training resulted in learning skills and information rapidly in a short time period.

Some participants specifically referred to accelerated learning while others spoke more generally of the rapid learning process. When speaking to the illustrative training method versus other trainings, one participant stated “nothing has the accelerated learning effect that [the trainer's] training has”. Other participants noted accelerated learning when responding to the question of “what stood out about their training”. For example, one respondent noted “I am able to achieve superior results, faster, and with less resources wasted than in conventional ways”. Another survey participant mentioned a specific exercise utilized in the training and indicated “I have never seen any single exercise produce such rapid and tangible results”.

Regarding the exceptional theme, survey respondents also spoke to the exceptional nature of the training as well as the instructor. Participants frequently referred to the instructor's high level of competence, the training exceeding expectations, and the life-changing nature (professional, personal, or both) of the training. The exceptional theme captures participants' descriptions of the training as going above and beyond normal expectations. One participant noted “[The trainer] remains head and shoulders above other instructors under whom I have trained and eclipses ‘industry standard’ courses altogether.” Another respondent also spoke directly to the trainer's abilities, stating “[The trainer] has a rare gift for facilitating the learning process in his students, I have never met a better practitioner”. In speaking to the exceptional nature of the training overall, a respondent reported, “All participants experienced dramatic improvement in targeted skills at the end of the course, often in excess of expectation”. Finally, several survey participants noted the life changing nature of their work with the trainer. One respondent noted, “I'm a different person. Training under these methods brought about a phase change in how I perceive myself and the world around me”. Another participant reported that the training was life changing and lifesaving “It was life changing. The training saved my life many times.”

Category 2: Lasting State Management.

Similar to interview participants, survey participants spoke to skills that they developed and maintained as a result of training with the trainer, as well as overall shifts in their state of being and state of mind. Consistent with interview findings, several themes emerged that represented lasting state change. As in the interviews, survey respondents spoke of lasting cognitive skills including memory, visual, and decision-making skills. The new integrated theme of psychophysiological state management is described herein, as well as nuances of the awareness theme as portrayed in the second part of the study.

With respect to the psychophysiological theme, while survey respondents spoke of state management frequently, they spoke of it more generally than the interview respondents who provided detailed in-depth information. Thus, amongst the survey qualitative data, the psychophysiological theme captures participants' reference to emotional (e.g. fear, anger, “emotion”) state management, physiological (i.e., reference to managing a physical response or state), state management, or general state management. Speaking generally about state management, one participant noted, “He basically introduced me to environmental awareness and state access”. Other respondents spoke understanding and managing their emotional response as a result of the training. For example, one participant stated, “Training with [the trainer] has helped me to understand the angry response from some customers and to understand what I am feeling when faced with this anger and how to manage both”. Another participant spoke about the impact of training in emotion management, “The emotional control allows me to see wider and come to good decisions faster”. In speaking to the physical state management that resulted from the training, a survey participant noted “[the training] made me conscious of the need to maintain 360 degree awareness and not to tunnel vision in stressful situations”.

The theme of awareness was well represented in the survey data as it had been amongst interviewees. The awareness theme encompasses participants' reference to environmental and self-awareness enhancement as a result of their training with the trainer. Compared to the interview data, survey respondents emphasized self-awareness to a larger extent. Specifically, survey findings highlighted the effect of increased confidence as a result of training with the trainer. According to one participant, what stood out most from the illustrative neural-based training was “The moment you recognize that you are capable of much more than you thought”. Other participants spoke about confidence while simultaneously noting environmental or situational awareness. One respondent reported gaining “better situational awareness on the job and more confident in thinking I could apply my skills under stress”. Another stated, “[I] feel far more confident intuitively adapting to a changing environment as needed”.

Category 3: Pervasive Impacts.

Consistent with the semi-structured interview data, the pervasive impacts category emerged from the survey data and represents the ways that the training extends beyond the type of scenarios trained for, as reported by trainees. Amongst survey participants, the pervasive impacts category retains the themes of ingrained automaticity and teaching/training others, and the nuances of these themes, as identified by survey participants, are described below. A new theme, broad applicability, emerged amongst survey participants and highlights the applicability of illustrative training method to various aspects of life.

As to the theme of ingrained automaticity, survey participants spoke to the powerful and pervasive impact of their training with the trainer, consistent with interview responses. As represented by the ingrained automaticity theme, respondents noted the ingrained longstanding nature of what they learned in their training with the trainer. Survey respondents emphasized the retention of skills and state change over time. In speaking to the impact of the training, one participant stated “I still use his techniques and advice on a daily basis” and another highlighted the longstanding influence, reporting “I remember it [the training] 20 years later. It definitely stood out.” Respondents also spoke to the connection between the immersive nature of the training and their retention. For example one respondent noted “the practical application of skills increased my retention level of skills presented” and another described the training as “more realistic, hands-on, and more easily retained”.

As demonstrated in the interview data, with respect to the theme of training/teaching others, survey participants spoke to the impact of the trainer's training on their own teaching and training of others. While one of the open-ended questions specifically addressed respondents' teaching and training of others, participants spoke about this theme throughout their responses to the five open-ended questions. When speaking to the overall impact of the training, one participant commented “The tools and methodology that I was exposed to through [the trainer] changed how, and why, I select and implement all of the work I do with clients.” Another participant noted the impact on teaching and training when answering the question about the impact of the training on professional life. This participant reported “improvements in all areas relating to the training and highly improved my teaching and instructing skills”.

The pervasive impacts category incorporates one new theme unique to the survey qualitative data. The broad applicability theme embodies participants' descriptions of the ways in which the training has impacted their daily lives and work with a variety of people. Survey participants emphasized the applicability of the training in day-to-day life. One participant claimed, “training with him [the trainer] gives a richer experience of daily life!” Another stated, “It [the training] was life changing for me. Skills learnt are used every day and taught to police”. Survey respondents also described the training as impacting their work with an array of individuals. For example, one respondent indicated, “I've been able to impart the concepts to others of a variety of backgrounds”. Another participant described the training as “valuable across the spectrum of clients and students that I interact with, from soccer moms to professional men and women under arms”.

Results: Part 2, Training-Specific Survey, Quantitative Analysis of Likert-Scale Items.

Following the t-tests, a significant effect of group was found for the PA-S subscale, t(64)=2.08, p<0.05, PTI-S subscale, t(32)=3.483, p<0.001, and PTI-NS subscale, t(32)=3.47, p<0.001, but not the PA-NS subscale or the PSS or STAI. For all significant between-group comparisons, average ratings were higher in the trainee group compared to the non-trainee group (see Table 4).

TABLE 4
Average ratings for each dependent variable by group.

| | PA-S | PA-NS | PTI-S* | PTI-NS* | PSS | STAI |
|---|---|---|---|---|---|---|
| Trainee | 36.52 | 32.00^{n.s.} | 39.12 | 31.65 | 32.53^{n.s.} | 35.64^{n.s.} |
| | (4.77) | (4.30) | (4.21) | (3.95) | (5.82) | (8.44) |
| Non-Trainee | 33.80 | 32.54^{n.s.} | 31.65 | 25.00 | 30.09^{n.s.} | 33.77^{n.s.} |
| | (5.73) | (4.74) | (7.78) | (6.83) | (6.43) | (5.95) |

Standard deviations are in parentheses.
The abbreviation n.s. denotes nonsignificant comparisons. PA-S = perceived ability stress/threat, PA-NS = perceived ability non-stress, PTI-S = perceived training impact stress/threat, PTI-NS = perceived training impact non-stress, PSS = Perceived Stress Survey STAI = State-Trait Anxiety Inventory,
*Indicates statistics based on randomly selected subsample of trainees to allow for equal sample sizes.

Discussion.

When integrating the findings from the semi-structured interviews and the training-specific survey, several common categories and themes regarding the illustrative training method emerged. In both the qualitative analyses of in-depth, semi-structured interviews and open-response items from the survey, the categories of: training experience, lasting state management, and pervasive impacts were common. For the training experience category, both studies had themes of immersive, dynamic, and unique in common, while survey responses also revealed themes of accelerated and exceptional. For the lasting state management category, cognitive skills and awareness were common; while semi-structured interviews had physiological and emotional state management as separate themes, the surveys combined these into one theme, psychophysiological. For the pervasive impacts category, ingrained automaticity and teaching/training others were common, while the semi-structured interviews had emotional release and healing compared to the surveys, which had broad applicability.

According to the quantitative survey data, trainees attribute these categories and themes embedded within the illustrative training method with positively impacting their situational awareness ability, including psychophysiological state management, stress inoculation, and cognitive skills, in both stressful/threatening and non-stressful situations. Further, trainees reported higher perceptions of situational awareness ability overall compared to the non-trainees. Although, trainees did not differ from non-trainees on the Perceived Stress Survey or Trait subscale of the State-Trait Anxiety Inventory.

The lasting state management category and related themes (i.e., ingrained automaticity, awareness, etc.) that emerged in the semi-structured interviews and were supported and modified in the survey responses, may indicate that situational awareness and state management may be overlapping and inseparable concepts. Indeed, other training protocols, such as Mindfulness-Based Mind Fitness Training (MMFT), that trains focused attention and awareness were found to decrease attentional lapses and increase stress recovery. The themes of psychophysiological state management, awareness, and cognitive skills appear to be embedded in an overarching “state management component”. Thus participant-identified themes are consistent yet may be semantically different. As such, the illustrative training method may be unique to other training protocols in that state management, stress inoculation, and cognitive skill training is embedded in the concept of successful situational awareness in a practical illustrative training method.

Survey participants strongly emphasized the role of accelerated learning when providing qualitative responses regarding their training experience. Additionally, they spoke about accelerated learning and immersive learning simultaneously at times, and also highlighted the importance of accelerated learning in the lasting and broad application of learned skills. For example one participant compared the illustrative training method to other trainings by noting that the “hands on and in the moment” training ““helped embed learning”. Another survey participant indicated that what stood out about the training was “That I don't understand how it works but it does work, very quickly, with little training. Also that the training once installed stayed with me for decades”.

Overall, trainees were found to perceive the installation protocol as positively impacting their situational awareness ability in stressful/threatening situations and non-stressful situations compared to the non-trainees. Qualitative analyses conducted with the interviews and on open-response survey items provided descriptive explanations for how the training protocol may have influenced situational awareness abilities. This exploratory research provides some foundational evidence that the illustrative training method is improving trainees' abilities in situational awareness and state management. Further, research suggests that this type of training design may facilitate accelerated learning.

### APPENDIX A

Introductory Protocol.

Could we first have you verbally confirm that you are aware that we are recording this conversation and have given us your consent to do so?

We would first like to thank you for the opportunity to speak with you today. As you've been informed, you were selected because of the training you have completed with Marcus Wynne. We're going to go over some logistics before we get started. Is that ok?

We want to remind you that recordings will be kept in a secure location accessible only to researchers on the project. Further, the recording will be destroyed following transcription. We also want to remind you that all information will be held confidential and that you are free to end participation at any time or refrain from answering any questions you are uncomfortable with. Finally, we will do our best not to use your name throughout the remainder of the interview to ensure confidentiality of your responses. If for any reason we slip and use your name it will be replaced with a pseudonym in the transcript.

We have planned this interview to last approximately 30-45 minutes. Although we have a few specific questions we'd like to ask, this interview is primarily unstructured and informal and many of our questions will evolve from the information that you provide.

We will do our best to cover as much as possible in the time allotted.

Do you have any questions about the logistics? We'd like to give you a little more information about the purpose of the interview and then hear from you.

This preliminary research is aimed at exploring training participants' experiences with training provided by Marcus Wynne. Our goal is to understand the training experience from the participant's perspective and to utilize this information to help guide further research into the mechanisms impacted by this training. There are no right or wrong answers, and this study is primarily exploratory. We want to encourage you to be as honest as possible, and ensure you that only the themes and data that emerge from these interviews will be shared outside of the primary research team. We greatly appreciate you taking the time to talk with us today.

Do you have any questions before we begin?

Personal Experiences of the Training.

1. For our first question, we would like to get a sense of what you recall from your training with Marcus. Please provide a brief summary of the training from your perspective.

Potential Probe: When did this training occur?

Potential Probe: Why were you doing the training? Potential Probe: How long did the training last?

2. Have you completed any other similar trainings not provided by Marcus? If so, can you briefly describe such training.

Potential Probe: When did this training occur? Potential Probe: Why were you doing the training? Potential Probe: How long did the training last?

3. From your perspective, how did your training with Marcus compare to the other trainings, you just described?

Potential Probe: Similarities? Potential Probe: Unique differences?

Member check [where you summarize what you have heard and ask respondent if your understanding is correct].

Now we are going to ask some more specific questions regarding your training experience with Marcus. Again, please be as honest as possible, and there is no pressure to respond if you do not remember or do not know the answer.

4. From your perspective, did the training with Marcus impact your daily behavior? If so, how?

Potential Probe: How did it impact your awareness of your environment? Potential Probe:

How did it impact your awareness of your self?

Potential Probe: How did it impact your responses to different events? Potential Probe: How did it impact your emotional experience?

5. Do you participate in any activities that could be perceived as stressful, such as when there is threat of injury or threat of life? If so, could you describe such activities?

6. From your perspective, did the training with Marcus impact your performance during these stressful activities? If so, how?

Potential Probe: Did others notice a change in your performance? If so, how?

Potential Probe: Have you adopted any of the aspects of what you learned in the training as part of your regular routine? If so, how?

Potential Probe: Have you taught any of the aspects of what you learned in the training to others? If so, please elaborate.

Further comments/reflections from interviewee? Final Member check.

Post Interview Comments and/or Observations:

### APPENDIX B

For the following statements, please use the scale below to rate your perceived ability in the areas described.

| 1 | 2 | 3 | 4 | 5 |
|---|---|---|---|---|
| poor | | average | | excellent |

1. Situational awareness of visual environment during times of stress or threat.

2. Situational awareness of visual environment during non-stressful times.

3. Situational awareness of sensory (all senses) environment during times of stress or threat.

4. Situational awareness of sensory (all senses) environment during non-stressful times.

5. Management of physiological (e.g., heart rate, adrenal response, mobility, etc.) reactions to stress or threat.

6. Management of physiological (e.g., heart rate, adrenal response, mobility, etc.) processes during non-stressful times.

7. Management of emotional (e.g., anger, aggression, fear, etc.) reactions to stress or threat.

8. Management of emotional (e.g., anger, aggression, fear, etc.) processes during non-stressful times.

9. Staying calm in the face of stress or threat.

10. Staying calm during non-stressful times.

11. Engaging higher order decision making during times of stress or threat.

12. Engaging higher order decision making during non-stressful times.

13. Engaging higher order memory processes during times of stress or threat.

14. Engaging higher order memory processes during non-stressful times.

15. Adjust perception of time to enhance processing of essential data during times of stress or threat.

16. Adjust perception of time to enhance processing of essential data during non-stressful times.

17. Coping with or recovering from experiencing a stressful or threatening event

If you indicated above that you completed training with Marcus Wynne please answer the following questions for that training. If you indicated above that you have not completed training with Marcus Wynne or one of his students, please answer the following questions for the other training that you identified above.

For the following statements, please use the scale below to rate the extent to which your training(s) has(have) impacted your abilities in the following areas.

| 1 | 2 | 3 | 4 | 5 |
|---|---|---|---|---|
| not at all | | somewhat | | extremely |
| influential | | influential | | influential |

1. Situational awareness of visual environment during times of stress or threat.

2. Situational awareness of visual environment during non-stressful times.

3. Situational awareness of sensory (all senses) environment during times of stress or threat.

4. Situational awareness of sensory (all senses) environment during non-stressful times.

5. Management of physiological (e.g., heart rate, adrenal response, mobility, etc.) reactions to stress or threat.

6. Management of physiological (e.g., heart rate, adrenal response, mobility, etc.) processes during non-stressful times.

7. Management of emotional (e.g., anger, aggression, fear, etc.) reactions to stress or threat.

8. Management of emotional (e.g., anger, aggression, fear, etc.) processes during non-stressful times.

9. Staying calm in the face of stress or threat.

10. Staying calm during non-stressful times.

11. Engaging higher order decision making during times of stress or threat.

12. Engaging higher order decision making during non-stressful times.

13. Engaging higher order memory processes during times of stress or threat.

14. Engaging higher order memory processes during non-stressful times.

15. Adjust perception of time to enhance processing of essential data during times of stress or threat.

16. Adjust perception of time to enhance processing of essential data during non-stressful times.

17. Coping with or recovering from experiencing a stressful or threatening event.

If you indicated above that you have completed a training with Marcus Wynne, please complete the following section:

The following questions are particularly important to the current research project and your thoughtful answers are valued and appreciated. Please take the time to respond as accurately and honestly as possible.

1. What stands out to you most about your experience training with Marcus Wynne (or if not trained directly by Marcus, the instructor you trained with in his brain-based state-management training methods)

2. How did your training experience with Marcus Wynne compare to other training experiences specific to your line of work?

3. How did your training with Marcus Wynne impact your performance in your current line of work?

4. How did your training with Marcus Wynne impact your life generally?

5. How has your personal training experience with Marcus Wynne impacted your teaching and/or training of others?

### Illustrative Embodiments

While the present disclosure is not so limited, an appreciation of various aspects of the disclosure will be gained through a discussion of illustrative embodiments herein. Various modifications of illustrative embodiments, as well as additional embodiments of the disclosure, will become apparent.

According to illustrative embodiment 1, a method for installing a critical skill path in a trainee, the critical skill path including one or more critical skills for successfully achieving an outcome in a stressful environment likely to elicit one or more stress response states in the trainee, includes: presenting a first instruction to the trainee prompting the trainee to perform a first task using an essential critical skill of the critical skill path; introducing the trainee into a first simulated environment, within a first brief period of time after presenting the first instruction, exposing the trainee to a first stressor and requiring the trainee to use the essential critical skill to successfully perform the first task, wherein the first stressor is designed to elicit a first stress response state in the trainee, and wherein the first simulated environment is designed to favor successful performance of the first task by the trainee in the first stress response state; presenting a second instruction to the trainee, within a second brief period of time after completing the first task, prompting the trainee to perform a second task using the essential critical skill and at least another critical skill of the critical skill path; introducing the trainee into a second simulated environment, within a third brief period of time after presenting the second instruction, exposing the trainee to a second stressor and requiring the trainee to use the essential critical skill and the at least another critical skill to successfully perform the second task, wherein the second stressor is designed to elicit a second stress response state in the trainee more stressful than the first stress response state, and wherein the second simulated environment is designed to favor successful performance of the second task by the trainee in the second stress response state; continuing to present one or more additional instructions to the trainee, within a brief period of time after completing an immediately preceding task, each additional instruction prompting the trainee to perform a task and to use a critical skill set including the critical skills used in the immediately preceding task and at least one additional critical skill, until all critical skills in the critical skill path have been used in performing the immediately preceding task; and continuing to introduce the trainee into one or more additional simulated environments within a brief period of time after presenting each additional instruction until all critical skill in the critical skill path have been used in performing the immediately preceding task, each additional simulated environment exposing the trainee to a stressor designed to elicit a stress response state in the trainee more stressful than the immediately preceding stress response state and requiring the trainee to use the corresponding critical skill set to successfully perform the task as instructed, wherein each additional simulated environment is designed to favor successful performance of the task, as instructed, by the trainee in the stress response state.

According to illustrative embodiment 2, a method includes the method of any other embodiment, wherein each brief period of time is less than about 10 minutes.

According to illustrative embodiment 3, a method includes the method of any other embodiment, wherein each brief period of time is less than about 5 minutes.

According to illustrative embodiment 4, a method includes the method of any other embodiment, wherein each brief period of time is less than about 3 minutes.

According to illustrative embodiment 5, a method includes the method of any other embodiment and further includes: presenting a basic instruction to the trainee, prior to presenting the first instruction, prompting the trainee to perform a basic task using a basic skill; and introducing the trainee into a basic simulated environment, within a brief period of time after presenting the basic instruction, exposing the trainee to a basic stressor designed to elicit a baseline stress response state in the trainee and requiring the trainee to use the basic skill and a basic stress management skill to manage the effects of the baseline stress response state to successfully perform the basic task, wherein the basic simulated environment is designed to favor successful performance of the basic task by the trainee in the baseline stress response state.

According to illustrative embodiment 6, a method includes the method of any other embodiment, and further includes: prompting the trainee, after introducing the trainee into a simulated environment, to use the basic stress management skill for managing effects of the one or more stress responses on the trainee to facilitate successful performance of the task as instructed.

According to illustrative embodiment 7, a method includes the method of any other embodiment, and further includes: prompting the trainee, after completing a corresponding task, to use the basic stress management skill for managing effects of the one or more stress responses on the trainee to facilitate successful performance of subsequent tasks.

According to illustrative embodiment 8, a method includes the method of any other embodiment, and further includes: presenting a stress recognition skill to the trainee, after introducing the trainee into a simulated environment, to recognize effects of one or more stress responses on the trainee.

According to illustrative embodiment 9, a method includes the method of any other embodiment, and further includes: presenting a stress recognition skill to the trainee to recognize effects of one or more stress responses on another person.

According to illustrative embodiment 10, a method includes the method of any other embodiment, wherein each critical skill of the critical skill path is presented to the trainee in a pre-determined order according to the critical skill path.

According to illustrative embodiment 11, a method includes the method of any other embodiment, wherein one or more critical skills of the critical skill path include a motor skill, a stress management skill, a cognitive strategy skill, or combinations thereof, wherein the cognitive strategy skill corresponds to making a critical decision related to use of the motor skill, use of the stress management skill, or both.

According to illustrative embodiment 12, a method includes the method of embodiment 11 or any other illustrative embodiment, wherein the each critical skill of the critical skill path includes a combination of at least two of a motor skill, a stress management skill, and a cognitive strategy skill.

According to illustrative embodiment 13, a method includes the method any other embodiment, and further includes: presenting another stress management skill to the trainee, after the trainee has completed at least three tasks, for managing effects of one or more stress responses on the trainee to facilitate successful performance of subsequent tasks.

According to illustrative embodiment 14, a method includes the method of any other embodiment and further includes: presenting a secondary instruction to the trainee prompting the trainee to perform a task using a supporting critical skill related to the essential critical skill of the critical skill path; and introducing the trainee into a secondary simulated environment, within a brief period of time after presenting the secondary instruction, exposing the trainee to a stressor designed to elicit a stress response state in the trainee and requiring the trainee to use the supporting skill and the related critical skill to successfully perform the task as instructed, wherein the secondary simulated environment is designed to favor successful performance of the task by the trainee as instructed in the stress response state.

According to illustrative embodiment 15, a method includes the method of any other embodiment, wherein a series of simulated environments require the trainee to use at least one cognitive strategy skill to make critical decisions on one or more choices to successfully perform the corresponding task, wherein each simulated environment of the series relative to an immediately preceding simulated environment requires the trainee to make more critical decisions.

According to illustrative embodiment 16, a method includes the method of any other embodiment and further includes: presenting an end-goal instruction to the trainee to achieve a goal in a comprehensive simulated environment; and introducing the trainee into the comprehensive simulated environment, within a brief period of time after presenting the end-goal instruction, exposing the trainee to one or more stressors designed to elicit a stress response state in the trainee and requiring the trainee to use all previously presented critical skills to successfully achieve the goal, wherein the comprehensive simulated environment is designed to favor successful achievement of the goal, as instructed, by the trainee in the one or more corresponding stress response states.

According to illustrative embodiment 17, a method includes the method of embodiment 16 or any other embodiment, wherein the comprehensive simulated environment requires the trainee to use all previously presented critical skills in a pre-determined order according to the critical skill path.

According to illustrative embodiment 18, a method includes the method of embodiment 16 or any other embodiment and further includes: prompting the trainee, after introducing the trainee into a comprehensive simulated environment, to use one or more previously presented skills to facilitate successful achievement of the goal.

According to illustrative embodiment 19, a method includes the method of embodiment 16 or any other embodiment and further includes: presenting an advanced series of end-goal instructions to the trainee including a first end-goal instruction, each end-goal instruction being presented within a brief period of time after attempting to achieve an immediately preceding goal after the first end-goal instruction, each end-goal instruction prompting the trainee to achieve a goal and to use at least one different advanced component of a critical skill of the critical skill path, wherein the advanced series of end-goal instructions as a whole prompts the trainee to use all advanced components of all critical skills in the critical skill path; introducing the trainee into an advanced series of comprehensive simulated environments, each corresponding to the advanced series of end-goal instructions and each being introduced within a brief period of time after presenting the corresponding end-goal instruction, each comprehensive simulated environment exposing the trainee to a stressor designed to elicit a stress response state in the trainee and requiring the trainee to use a combination of skills including the at least one different advanced component and optionally to make one or more critical decisions to successfully achieve the goal as instructed, wherein each comprehensive simulated environment in the advanced series is designed to be more difficult for the trainee relative to an immediately preceding comprehensive simulated environment by at least one of requiring the trainee to use a different combination of skills, exposing the trainee to a stressor designed to elicit a more stressful response state, and requiring the trainee to make more critical decisions, wherein the advanced series of comprehensive simulated environments as a whole requires the trainee to use all advanced components of all critical skill in the critical skill path.

According to illustrative embodiment 20, a method includes the method of embodiment 19 or any other embodiment, wherein each comprehensive simulated environment requires the trainee to use the combination of one or more skills in a pre-determined order according to the critical skill path.

According to illustrative embodiment 21, a method includes the method of embodiment 19 or any other embodiment and further includes: presenting an escalating series of end-goal instructions to the trainee including a first end-goal instruction, each end-goal instruction being presented within a brief period of time after attempting to achieve an immediately preceding goal after the first end-goal instruction of the escalating series, each end-goal instruction prompting the trainee to achieve a goal, wherein the escalating series of end-goal instructions as a whole prompts the trainee to use all previously presented critical skills; introducing the trainee into an escalating series of comprehensive simulated environments, each corresponding to the escalating series of end-goal instructions and each being introduced within a brief period of time after presenting the corresponding end-goal instruction, each comprehensive simulated environment exposing the trainee to a stressor designed to elicit a stress response state in the trainee and requiring the trainee to use a combination of previously presented skills and to make one or more critical decisions to successfully achieve the goal as instructed, wherein each comprehensive simulated environment in the escalating series is designed to maintain and escalate the stress response state in the trainee relative to an immediately preceding comprehensive simulated environment by at least one of requiring the trainee to use a different combination of skills, exposing the trainee to a stressor designed to elicit a more stressful response state, and requiring the trainee to make more critical decisions, wherein the escalating series of comprehensive simulated environments as a whole requires the trainee to use all previously presented critical skills.

According to illustrative embodiment 22, a method includes the method of embodiment 21 or any other embodiment and further includes: prompting the trainee, after introducing the trainee into a comprehensive simulated environment of the escalating series, to use one or more previously presented skills to facilitate successful achievement of the goal.

According to illustrative embodiment 23, a method includes the method of embodiment 21 or any other embodiment and further includes: presenting a severe series of end-goal instructions to the trainee including a first end-goal instruction, each end-goal instruction being presented within a brief period of time after attempting to achieve an immediately preceding goal after the first end-goal instruction of the severe series, each end-goal instruction prompting the trainee to achieve a goal, wherein the severe series of end-goal instructions as a whole prompts the trainee to use all previously presented critical skills; introducing the trainee into a severe series of comprehensive simulated environments, each corresponding to the severe series of end-goal instructions and each being introduced within a brief period of time after presenting the corresponding end-goal instruction, each comprehensive simulated environment exposing the trainee to a stressor designed to elicit a severe stress response state in the trainee greater than any previously elicited stress response state and requiring the trainee to use a combination of previously presented skills and to make multiple critical decisions to successfully achieve the goal as instructed, wherein each comprehensive simulated environment in the severe series is different than other comprehensive simulated environment in the severe series by at least one of requiring the trainee to use a different combination of skills, exposing the trainee to a different stressor designed to elicit a severe stress response state, and requiring the trainee to make different critical decisions, wherein the severe series of comprehensive simulated environments as a whole requires the trainee to use all previously presented critical skills.

According to illustrative embodiment 24, a method includes the method of embodiment 23 or any other embodiment and further includes: prompting the trainee, within a brief period of time after identifying a failure of the trainee to achieve a present goal in a comprehensive simulated environment of the severe series, to identify one or more previously presented skills that would have facilitated successful achievement of the present goal; presenting a subsequent end-goal instruction in the severe series to the trainee prompting the trainee to achieve a subsequent goal within a brief period of time after the trainee identifies one or more previously presented skills that would have facilitated successful achievement of the present goal; and introducing the trainee a subsequent comprehensive simulated environment in the severe series corresponding to the subsequent end-goal instruction within a brief period of time after presenting the subsequent end-goal instruction.

According to illustrative embodiment 25, a method includes any method of embodiments 16, 19, and 23 or any other embodiment, wherein all previously presented critical skills include all critical skills in the critical skill path.

According to illustrative embodiment 26, a method includes the method of embodiment 23 or any other embodiment and further includes: presenting one or more stress management skills designed to reduce the effects of a stress response state on the trainee within a brief period of time after completing a series of simulated environments, each designed to elicit a corresponding stress response state, wherein the one or more stress management skills are presented in a pre-determined order according to a pre-determined sequence of elicited stress response states defined by the series of simulated environments.

According to illustrative embodiment 27, a method includes the method of embodiment 23 or any other embodiment and further includes: presenting one or more cognitive practice skills to practice previously presented skills, wherein the one or more cognitive skills include one or more of: an active visualization skill, a mental rehearsal skill, an active stress management skill, a replay skill, and a recall skill.

According to illustrative embodiment 28, a method includes the method of embodiment 27 or any other embodiment and further includes prompting the trainee to use previously presented skills in a pre-determined sequence of a previously introduced simulated environment.

According to illustrative embodiment 29, a method includes the method of embodiment 1 or any other embodiment and further includes determining a sequence of critical skills for the critical skill path to successfully achieve an outcome in a stressful environment.

According to illustrative embodiment 30, a method includes the method of embodiment 1 or any other claim, wherein the set of experiences defines an order different than an order of the critical skill path.

According to illustrative embodiment 31, a method for installing a critical skill path in a trainee, the critical skill path comprising one or more critical skills for successfully achieving an outcome in a stressful environment likely to elicit one or more stress response states in the trainee, includes: (a) presenting a basic instruction to the trainee prompting the trainee to perform a basic task using a basic skill related to the critical skill path; (b) introducing the trainee into a basic simulated environment, within a brief period of time after presenting the basic instruction, exposing the trainee to a basic stressor and requiring the trainee to use the basic skill to successfully perform the basic task, wherein the basic stressor is designed to elicit a baseline stress response state in the trainee, and wherein the basic simulated environment is designed to favor successful performance of the basic task by the trainee in the basic stress response state; (c) determining whether the trainee has reached at least a threshold level of stress after completing the basic task in the basic simulated environment; (i) if the trainee is determined to have reached at least the threshold level of stress, proceed to step (d); (ii) if the trainee is determined to have not reached at least the threshold level of stress, introduce the trainee into a subsequent simulated environment, within another brief period of time after the trainee completed the previous task, exposing the trainee to a more severe stressor and requiring the trainee to use the basic skill to successfully perform the basic task, wherein the subsequent stressor is designed to elicit a more severe stress response state in the trainee relative to the stressor in the previous simulated environment, and wherein the subsequent simulated environment is designed to favor successful performance of the basic task by the trainee in the baseline stress response state, and repeat step (c); and (d) presenting a core instruction to the trainee, within another brief period of time after completing the basic task and having reached at least the threshold level of stress, prompting the trainee to perform a core task using an essential critical skill of the critical skill path.

According to illustrative embodiment 32, a method for developing a learning under stress model comprises introducing a trainee into a simulated environment exposing the trainee to a stressor and requiring the trainee to use a skill to successfully perform a task, wherein the stressor is designed to elicit a stress response state in the trainee, and wherein the simulated environment is designed to favor successful performance of the task by the trainee in the stress response state; monitoring the trainee for a marker related to the simulated environment; and determining a critical marker for the learning under stress model in response to the marker monitoring step.

According to illustrative embodiment 33, a method comprises the method of illustrative embodiment 1 or any other embodiment, wherein the critical marker determining step comprises determining whether the marker represents at least one of learning the skill, successful performance of the skill, a psychophysiological state of the trainee, and a change in the psychophysiological state of the trainee.

According to illustrative embodiment 34, a method comprises the method of illustrative embodiment 33 or any other embodiment, further comprising determining an associated critical skill along a critical skill path for the critical marker.

According to illustrative embodiment 35, a method comprises the method of illustrative embodiment 33 or any other embodiment, further comprising determining a critical marker path comprising a sequence of critical markers associated with a critical skill path in response to introducing a series of simulated environments requiring the trainee to use a series of critical skills along the critical skill path.

According to illustrative embodiment 36, a method comprises the method of illustrative embodiment 1 or any other embodiment, wherein the critical marker is associated with an environmental factor defined by the simulated environment that is likely to influence the presence or absence of the critical marker in the trainee.

According to illustrative embodiment 37, a method comprises the method of illustrative embodiment 36 or any other embodiment, wherein the environmental factor is selected from at least one of: a task, an objective, a stressor, a threat, a protectee, a visual presentation, an aural presentation, an electrical stimulation, an administered psychophysiological agent, a required critical choice, a required critical skill, a required critical skill aspect, coaching, a type thereof, a level thereof, and a number thereof.

According to illustrative embodiment 38, a method comprises the method of illustrative embodiment 32 or any other embodiment, wherein the simulated environment is designed to subconsciously install the skill in the trainee.

According to illustrative embodiment 39, a method comprises the method of illustrative embodiment 32 or any other embodiment, comprising connecting the trainee to an automated marker monitoring apparatus configured to monitor the marker in the trainee.

According to illustrative embodiment 40, a method comprises the method of illustrative embodiment 32 or any other embodiment, wherein the critical marker is determined in response to a marker value selected from at least one of a marker presence value, an absolute marker value, a marker change value, a marker duration value, and a relationship to another marker value.

According to illustrative embodiment 41, a method comprises the method of illustrative embodiment 32 or any other embodiment, wherein the critical marker represents a change in the psychophysiological state of the trainee associated with learning the skill.

According to illustrative embodiment 42, a method comprises the method of illustrative embodiment 41 or any other embodiment, comprising determining the psychophysiological state change by comparing a before-simulation marker value and an after-simulation marker value.

According to illustrative embodiment 43, a method comprises the method of illustrative embodiment 32 or any other embodiment, wherein the critical marker represents the psychophysiological state of the trainee associated with using the skill.

According to illustrative embodiment 44, a method comprises the method of illustrative embodiment 32 or any other embodiment, wherein the marker is of a type selected from at least one of an external cue and an internal biomarker.

According to illustrative embodiment 45, a method comprises the method of illustrative embodiment 32 or any other embodiment, wherein the marker monitoring step comprises monitoring a specific marker in response to the skill required to be used in the simulated environment.

According to illustrative embodiment 46, a method comprises the method of illustrative embodiment 32 or any other embodiment, comprising installing the skill in a subsequent trainee by introducing the subsequent trainee into a subsequent simulated environment and monitoring for the presence of the critical marker in the subsequent trainee.

According to illustrative embodiment 47, a method comprises the method of illustrative embodiment 46 or any other embodiment, comprising calibrating a training apparatus to monitor for the presence of the critical marker in the subsequent trainee, wherein the calibrated training apparatus is configured to present one or more environmental factors to the subsequent trainee in one or more simulated environments.

According to illustrative embodiment 48, a method comprises the method of illustrative embodiment 32 or any other embodiment, wherein a training apparatus performs the steps of presenting an environmental factor of the simulated environment with a training apparatus, the marker monitoring step, and the critical marker determining step.

According to illustrative embodiment 49, a method comprises the method of illustrative embodiment 32 or any other embodiment, wherein the method defines a critical skill path comprising one or more critical skills for successfully achieving an outcome in a stressful environment likely to elicit one or more stress response states in the trainee, each of the critical skills comprising at least one critical skill aspect selected from a motor skill, a cognitive strategy skill, a stress management skill, a supporting skill, and a skill component. The method further comprises presenting a first instruction to the trainee prompting the trainee to perform the task using a critical skill aspect of the skill, wherein the skill is defined as a critical skill of the critical skill path, the task is defined as a first task; presenting a second instruction to the trainee, within a second brief period of time after completing the first task, prompting the trainee to perform a second task using the critical skill aspect and another critical skill aspect, the another skill aspect being related to the same critical skill or a different critical skill; introducing the trainee into a second simulated environment, within a third brief period of time after presenting the second instruction, exposing the trainee to a second stressor and requiring the trainee to use the critical skill aspect and the another critical skill aspect to successfully perform the second task, wherein the second stressor is designed to elicit a second stress response state in the trainee more stressful than the first stress response state, and wherein the second simulated environment is designed to favor successful performance of the second task by the trainee in the second stress response state; continuing to present one or more additional instructions to the trainee, within a brief period of time after completing an immediately preceding task, each additional instruction prompting the trainee to perform a task and to use a critical skill set including the critical skills used in the immediately preceding task and at least one additional critical skill aspect, until all critical skills related to the critical skill path have been used in performing the immediately preceding task; and continuing to introduce the trainee into one or more additional simulated environments within a brief period of time after presenting each additional instruction until all critical skills in the critical skill path have been used in performing the immediately preceding task, each additional simulated environment exposing the trainee to a stressor designed to elicit a stress response state in the trainee more stressful than the immediately preceding stress response state and requiring the trainee to use the corresponding critical skill set to successfully perform the task as instructed, wherein each additional simulated environment is designed to favor successful performance of the task, as instructed, by the trainee in the stress response state;

According to illustrative embodiment 50, a method of using a learning under stress model comprises introducing a trainee into a simulated environment exposing the trainee to an environmental factor designed to facilitate the presence of a critical marker, the environ mental factor including a stressor and requiring the trainee to use a skill to successfully perform a task, wherein the stressor is designed to elicit a stress response state in the trainee, and wherein the simulated environment is designed to favor successful performance of the task by the trainee in the stress response state; monitoring the trainee for a critical marker defined in response to monitoring a previous trainee; determining whether the critical marker is present or absent in response to the monitoring step; and determining a subsequent environmental factor in response to the step of determining the absence of the critical marker, the subsequent environmental factor being different than the environmental factor and designed to further facilitate the presence of the critical marker.

According to illustrative embodiment 51, a method comprises the method of illustrative embodiment 50 or any other embodiment, wherein the critical marker determining step comprises comparing a critical marker value associated with the critical marker to a corresponding marker target.

According to illustrative embodiment 52, a method comprises the method of illustrative embodiment 51 or any other embodiment, comprising calibrating the corresponding marker target in response to a characteristic related the trainee selected from at least one of a prior experience, a psychophysiological state response to a specific stressor, a psychophysiological state response to a specific skill, a measured baseline marker, a cognitive trait, and a physiological trait.

According to illustrative embodiment 53, a method comprises the method of illustrative embodiment 50 or any other embodiment, comprising introducing the trainee into a subsequent simulated environment in response to the determining that the critical marker is absent, wherein the subsequent simulated environment has the subsequent environmental factor selected in response to the subsequent environmental factor determining step.

According to illustrative embodiment 54, a method comprises the method of illustrative embodiment 53 or any other embodiment, wherein the subsequent simulated environment is designed to require the use of the skill.

According to illustrative embodiment 55, a method comprises the method of illustrative embodiment 53 or any other embodiment, wherein the subsequent simulated environment is introduced within a brief period of time after the simulated environment.

According to illustrative embodiment 56, a method comprises the method of illustrative embodiment 50 or any other embodiment, comprising continuing to introduce the trainee into additional simulations until the critical marker is determined to be present, wherein each additional simulation defines another environmental factor different than a preceding environmental factor, the another environmental factor being designed to facilitate the presence of the critical marker.

According to illustrative embodiment 57, a method comprises the method of illustrative embodiment 50 or any other embodiment, comprising introducing the trainee into a subsequent simulated environment in response to the step of determining that the critical marker is present, wherein the subsequent simulated environment is designed to require the use of a different skill.

According to illustrative embodiment 58, a method comprises the method of illustrative embodiment 50 or any other embodiment, comprising adjusting the simulated environment in response to the absence of the critical marker, wherein the subsequent environmental factor is introduced before the trainee completes the simulated environment.

According to illustrative embodiment 59, a method comprises the method of illustrative embodiment 50 or any other embodiment, wherein the simulated environment is designed to require the use of at least three skills including the skill.

According to illustrative embodiment 60, an apparatus for developing a learning under stress model for installing a critical skill with a critical skill presentation comprises a data storage configured to store one or more parameters of the learning under stress model associated with the critical skill including at least simulated environmental factor and a critical marker target; a sensor configured to provide a marker signal representing a specific measured marker of a trainee after being introduced into an environment exposing the trainee to a simulated environmental factor designed to facilitate subconscious installation of the critical skill in the trainee for achieving a desired outcome under stress; and a data processor. The data processor is configured to determine whether the marker signal is associated with achievement of the desired outcome; determine whether the specific measured marker is a critical marker for the learning under stress model in response to the marker signal being associated with achievement of the desired outcome; and determine the critical marker target for storage in the data storage in response to determining that the measured marker is a critical marker, the critical marker target representing the a critical marker value associated with achievement of the desired outcome.

According to illustrative embodiment 61, an apparatus comprises the apparatus of illustrative embodiment 60 or any other embodiment, wherein the data storage is configured to store as the one or more parameters of the learning under stress model at least one of: an environmental marker, a psychophysiological state, a critical skill presentation designed to introduce the critical skill to the individual for subconscious installation, and a desired outcome marker representing achievement of a desired outcome in the environment.

According to illustrative embodiment 62, an apparatus comprises the apparatus of illustrative embodiment 61 or any other embodiment, wherein at least some of the one or more parameters of the learning under stress model are associated in the data storage.

According to illustrative embodiment 63, an apparatus for installing a critical skill with a critical skill presentation according to a learning under stress model comprises a data storage configured to store one or more parameters of the learning under stress model associated with the critical skill including at least a simulated environmental factor, a critical marker target for a critical marker, and a desired outcome marker representing achievement of a desired outcome in a specific environment; an installation controller configured to expose the trainee to the simulated environmental factor of the specific environment designed to facilitate subconscious installation of the critical skill in the trainee for achieving the desired outcome under stress; a sensor configured to provide a critical marker signal representing a measured value of a critical marker after introducing the critical skill presentation to the trainee; and a data processor. The data processor is configured to determine whether the critical marker is present or absent in response to the critical marker signal and the critical marker target in the data storage; and determine a subsequent simulated environmental factor for exposure to the trainee in response to the critical marker being absent, the subsequent simulated environmental factor being different than the simulated environmental factor in at least one of type, level, and number. The installation controller is further configured to expose the trainee to the subsequent simulated environmental factor in response to the data processor determining the subsequent simulated environmental factor.

According to illustrative embodiment 64, an apparatus comprises the apparatus of illustrative embodiment 63 or any other embodiment, wherein the installation controller comprises a virtual reality interface configured to provide a simulated environmental factor, the virtual reality interface including at least one of an image generator, a sound generator, a motion generator, and a haptic-sensation generator.

According to illustrative embodiment 65, an apparatus comprises the apparatus of illustrative embodiment 63 or any other embodiment, wherein the installation controller comprises an electrical stimulator configured to provide a simulated environmental factor, the electrical stimulator including at least one of an electrode and an electrical signal generator.

According to illustrative embodiment 66, an apparatus comprises the apparatus of illustrative embodiment 63 or any other embodiment, wherein the installation controller comprises an agent administrator configured to provide a simulated environmental factor, the agent administrator including a psychophysiological agent and an administering connection to the trainee.

According to illustrative embodiment 67, an apparatus comprises the apparatus of illustrative embodiment 63 or any other embodiment, wherein the data processor is configured to determine a psychophysiological state of the trainee in response to the critical marker signal, wherein the subsequent simulated environmental factor is further determined in response to the psychophysiological state.

According to illustrative embodiment 68, an apparatus for using a learning under stress model to adaptively install a critical skill of an individual exposed to a stressful environment having one or more environmental factors comprises a data storage configured to store one or more parameters of the learning under stress model associated with the critical skill including at least: an environmental marker corresponding to a specific environmental factor, a critical marker target corresponding to a specific critical marker, a simulated environmental factor designed to facilitate subconscious installation of the critical skill in the individual for achieving a desired outcome under stress, and a critical skill presentation designed to introduce the critical skill to the individual for subconscious installation; an installation controller configured to expose the individual to the simulated environmental factor to facilitate installation of the critical skill in the individual and to present the critical skill presentation to the individual in the stressful environment; a marker sensor configured to provide a critical marker signal representing a measurement of the specific critical marker of the individual; an environmental sensor configured to provide an environmental signal representing a measurement of the specific environmental factor; and a data processor. The data processor is configured to determine whether to install the critical skill in response to the environmental signal and the environmental marker in the data storage; determine whether to present the critical skill presentation to the trainee in response to determining to install the critical skill; and determine whether to expose the trainee to the simulated environmental factor to facilitate subconscious installation of the critical skill in response to determining to install the critical skill and the critical marker signal. The installation controller is further configured to expose the trainee to the simulated environmental factor and to present the critical skill presentation in response to the respective data processor determinations.

According to illustrative embodiment 69, an apparatus comprises the apparatus of illustrative embodiment 68 or any other embodiment, wherein the data processor is further configured to determine whether the desired outcome has been achieved in response to the desired outcome marker in the data storage and at least one of the critical marker signal and the environmental signal; and update the one or more parameters of the learning under stress model in the data storage.

According to illustrative embodiment 70, an apparatus comprises the apparatus of illustrative embodiment 68 or any other embodiment, wherein the critical skill is a cognitive strategy skill for deciding a critical choice corresponding to the specific environmental factor.

Thus, embodiments of the SYSTEM, APPARATUS, AND METHOD OF ACCELERATED TRAINING FOR PERFORMANCE UNDER STRESS are disclosed. All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof. The disclosed embodiments are presented for purposes of illustration and not limitation.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein throughout, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

What is claimed is:

1. A method for installing a critical skill path in a trainee, the critical skill path comprising one or more critical skills for successfully achieving an outcome in a stressful environment likely to elicit one or more stress response states in the trainee, comprising:
developing a series of experiences, each including an instruction and a simulation, designed to subconsciously install the critical skill path in the trainee;
presenting a first instruction to the trainee prompting the trainee to perform a first task using an essential critical skill of the critical skill path;
introducing the trainee into a first simulated environment, within a first brief period of time after presenting the first instruction, exposing the trainee to a first stressor and requiring the trainee to use the essential critical skill to successfully perform the first task, wherein the first stressor is designed to elicit a first stress response state in the trainee, and wherein the first simulated environment is designed to favor successful performance of the first task by the trainee in the first stress response state;
presenting a second instruction to the trainee, within a second brief period of time after completing the first task, prompting the trainee to perform a second task using the essential critical skill and at least another critical skill of the critical skill path;
introducing the trainee into a second simulated environment, within a third brief period of time after presenting the second instruction, exposing the trainee to a second stressor and requiring the trainee to use the essential critical skill and the at least another critical skill to successfully perform the second task, wherein the second stressor is designed to elicit a second stress response state in the trainee more stressful than the first stress response state, and wherein the second simulated environment is designed to favor successful performance of the second task by the trainee in the second stress response state;
continuing to present one or more additional instructions to the trainee, within a brief period of time after completing an immediately preceding task, each additional instruction prompting the trainee to perform a task and to use a critical skill set including the critical skills used in the immediately preceding task and at least one additional critical skill, until all critical skills in the critical skill path have been used in performing the immediately preceding task; and
continuing to introduce the trainee into one or more additional simulated environments within a brief period of time after presenting each additional instruction until all critical skill in the critical skill path have been used in performing the immediately preceding task, each additional simulated environment exposing the trainee to a stressor designed to elicit a stress response state in the trainee more stressful than the immediately preceding stress response state and requiring the trainee to use the corresponding critical skill set to successfully perform the task as instructed, wherein each additional simulated environment is designed to favor successful performance of the task, as instructed, by the trainee in the stress response state.

2. The method of claim 1, comprising:
presenting a basic instruction to the trainee, prior to presenting the first instruction, prompting the trainee to perform a basic task using a basic skill; and
introducing the trainee into a basic simulated environment, within a brief period of time after presenting the basic instruction, exposing the trainee to a basic stressor designed to elicit a baseline stress response state in the trainee and requiring the trainee to use the basic skill and a basic stress management skill to manage the effects of the baseline stress response state to successfully perform the basic task, wherein the basic simulated environment is designed to favor successful performance of the basic task by the trainee in the baseline stress response state.

3. The method of claim 1, comprising:
presenting a secondary instruction to the trainee prompting the trainee to perform a task using a supporting critical skill related to the essential critical skill of the critical skill path; and
introducing the trainee into a secondary simulated environment, within a brief period of time after presenting the secondary instruction, exposing the trainee to a stressor designed to elicit a stress response state in the trainee and requiring the trainee to use the supporting skill and the related critical skill to successfully perform the task as instructed, wherein the secondary simulated environment is designed to favor successful performance of the task by the trainee as instructed in the stress response state.

4. The method of claim 1, comprising:
presenting an advanced series of end-goal instructions to the trainee including a first end-goal instruction, each end-goal instruction being presented within a brief period of time after attempting to achieve an immediately preceding goal after the first end-goal instruction, each end-goal instruction prompting the trainee to achieve a goal and to use at least one different advanced component of a critical skill of the critical skill path, wherein the advanced series of end-goal instructions as a whole prompts the trainee to use all advanced components of all critical skills in the critical skill path;
introducing the trainee into an advanced series of comprehensive simulated environments, each corresponding to the advanced series of end-goal instructions and each being introduced within a brief period of time after presenting the corresponding end-goal instruction, each comprehensive simulated environment exposing the trainee to a stressor designed to elicit a stress response state in the trainee and requiring the trainee to use a combination of skills including the at least one different advanced component and optionally to make one or more critical decisions to successfully achieve the goal as instructed, wherein each comprehensive simulated environment in the advanced series is designed to be more difficult for the trainee relative to an immediately preceding comprehensive simulated environment by at least one of requiring the trainee to use a different combination of skills, exposing the trainee to a stressor designed to elicit a more stressful response state, and requiring the trainee to make more critical decisions, wherein the advanced series of comprehensive simulated environments as a whole requires the trainee to use all advanced components of all critical skill in the critical skill path.

5. The method of claim 4, comprising:
presenting an escalating series of end-goal instructions to the trainee including a first end-goal instruction, each end-goal instruction being presented within a brief period of time after attempting to achieve an immediately preceding goal after the first end-goal instruction of the escalating series, each end-goal instruction prompting the trainee to achieve a goal, wherein the escalating series of end-goal instructions as a whole prompts the trainee to use all previously presented critical skills; and
introducing the trainee into an escalating series of comprehensive simulated environments, each corresponding to the escalating series of end-goal instructions and each being introduced within a brief period of time after presenting the corresponding end-goal instruction, each comprehensive simulated environment exposing the trainee to a stressor designed to elicit a stress response state in the trainee and requiring the trainee to use a combination of previously presented skills and to make one or more critical decisions to successfully achieve the goal as instructed, wherein each comprehensive simulated environment in the escalating series is designed to maintain and escalate the stress response state in the trainee relative to an immediately preceding comprehensive simulated environment by at least one of requiring the trainee to use a different combination of skills, exposing the trainee to a stressor designed to elicit a more stressful response state, and requiring the trainee to make more critical decisions, wherein the escalating series of comprehensive simulated environments as a whole requires the trainee to use all previously presented critical skills.

6. A method of using a learning under stress model comprising:
introducing a trainee into a simulated environment exposing the trainee to an environmental factor designed to facilitate the presence of a critical marker, the environ mental factor including a stressor and requiring the trainee to use a skill to successfully perform a task, wherein the stressor is designed to elicit a stress response state in the trainee, and wherein the simulated environment is designed to favor successful performance of the task by the trainee in the stress response state;
monitoring the trainee for a critical marker defined in response to monitoring a previous trainee;
determining whether the critical marker is present or absent in response to the monitoring step; and
determining a subsequent environmental factor in response to the step of determining the absence of the critical marker, the subsequent environmental factor being different than the environmental factor and designed to further facilitate the presence of the critical marker.

7. The method of claim 6, wherein the critical marker determining step comprises comparing a critical marker value associated with the critical marker to a corresponding marker target.

8. The method of claim 7, comprising calibrating the corresponding marker target in response to a characteristic related the trainee selected from at least one of a prior experience, a psychophysiological state response to a specific stressor, a psychophysiological state response to a specific skill, a measured baseline marker, a cognitive trait, and a physiological trait.

9. The method of claim 6, comprising introducing the trainee into a subsequent simulated environment in response to the determining that the critical marker is absent, wherein the subsequent simulated environment has the subsequent environmental factor selected in response to the subsequent environmental factor determining step.

10. The method of claim 9, wherein the subsequent simulated environment is designed to require the use of the skill.

11. The method of claim 9, wherein the subsequent simulated environment is introduced within a brief period of time after the simulated environment.

12. The method of claim 6, comprising continuing to introduce the trainee into additional simulations until the critical marker is determined to be present, wherein each additional simulation defines another environmental factor different than a preceding environmental factor, the another environmental factor being designed to facilitate the presence of the critical marker.

13. The method of claim 6, comprising introducing the trainee into a subsequent simulated environment in response to the step of determining that the critical marker is present, wherein the subsequent simulated environment is designed to require the use of a different skill.

14. The method of claim 6, comprising adjusting the simulated environment in response to the absence of the critical marker, wherein the subsequent environmental factor is introduced before the trainee completes the simulated environment.

15. The method of claim 6, wherein the simulated environment is designed to require the use of at least three skills including the skill.

16. An apparatus for installing a critical skill with a critical skill presentation according to a learning under stress model comprising:
a data storage configured to store one or more parameters of the learning under stress model associated with the critical skill including at least a simulated environmental factor, a critical marker target for a critical marker, and a desired outcome marker representing achievement of a desired outcome in a specific environment;
an installation controller configured to expose the trainee to the simulated environmental factor of the specific environment designed to facilitate subconscious installation of the critical skill in the trainee for achieving the desired outcome under stress;
a sensor configured to provide a critical marker signal representing a measured value of a critical marker after introducing the critical skill presentation to the trainee; and
a data processor configured to:
determine whether the critical marker is present or absent in response to the critical marker signal and the critical marker target in the data storage; and
determine a subsequent simulated environmental factor for exposure to the trainee in response to the critical marker being absent, the subsequent simulated environmental factor being different than the simulated environmental factor in at least one of type, level, and number;
wherein the installation controller is further configured to expose the trainee to the subsequent simulated environmental factor in response to the data processor determining the subsequent simulated environmental factor.

17. The apparatus of claim 16, wherein the installation controller comprises a virtual reality interface configured to provide a simulated environmental factor, the virtual reality interface including at least one of an image generator, a sound generator, a motion generator, and a haptic-sensation generator.

18. The apparatus of claim 16 or any other claim, wherein the installation controller comprises an electrical stimulator configured to provide a simulated environmental factor, the electrical stimulator including at least one of an electrode and an electrical signal generator.

19. The apparatus of claim 16, wherein the installation controller comprises an agent administrator configured to provide a simulated environmental factor, the agent administrator including a psychophysiological agent and an administering connection to the trainee.

20. The apparatus of claim 16, wherein the data processor is configured to determine a psychophysiological state of the trainee in response to the critical marker signal, wherein the subsequent simulated environmental factor is further determined in response to the psychophysiological state.
